(19) United States
(12) Patent Application Publication
(10) Pub. No.: US 2026/0102085 A1
(43) Pub. Date: Apr. 16, 2026

(54) WRIST AND FINGER WORN PULSE OXIMETRY SYSTEM

(71) Applicant: MASIMO CORPORATION, Irvine, CA (US)

(72) Inventors: Ammar Al-Ali, San Juan Capistrano, CA (US); Chad A. DeJong, Los Angeles, CA (US); Stephen Scruggs, Newport Beach, CA (US); James Ford Schramm, Huntington Beach, CA (US); Richard Priddell, Irvine, CA (US); Sujin Hwang, Irvine, CA (US); Mitchell Lloyd Ambrosini, Laguna Niguel, CA (US)

(21) Appl. No.: 19/331,892

(22) Filed: Sep. 17, 2025

Related U.S. Application Data
(63) Continuation of application No. 18/146,310, filed on Dec. 23, 2022, now Pat. No. 12,440,128.
(60) Provisional application No. 63/296,725, filed on Jan. 5, 2022.
(60) Provisional application No. 63/316,625, filed on Mar. 4, 2022.
(60) Provisional application No. 63/375,191, filed on Sep. 9, 2022.

(51) Int. Cl. A61B 5/1455; A61B 5/00

(52) U.S. Cl. CPC A61B 5/14552; A61B 5/6826; A61B 2560/0456

(57) ABSTRACT

A pulse oximetry system includes a wrist portion configured for placement on a wrist of a subject, the wrist portion having a first component and a second component configured to removably secure to one another. The wrist portion can include emitter(s) and detector(s) operably positioned by the wrist portion. In some implementations, the pulse oximetry system further includes a ring member configured to secure around the subject's finger and operably position emitter(s) and detector(s) and a cable connected to the wrist portion in electrical communication with the emitter(s) and the detector(s) of the ring member and configured to transmit the signal(s) from the detector(s) to the wrist portion. The system includes a battery and hardware processor(s) configured to receive and process signal(s) outputted by the detector(s) to determine physiological parameter(s) of the subject.

### INCORPORATION BY REFERENCE TO ANY PRIORITY APPLICATIONS

The present application is a continuation of U.S. application Ser. No. 18/146,310, filed Dec. 23, 2022, which claims priority to U.S. Provisional Application No. 63/296,725, filed Jan. 5, 2022, U.S. Provisional Application No. 63/316,625, filed Mar. 4, 2022, and U.S. Provisional Application No. 63/375,191, filed Sep. 9, 2022. All of the above-listed applications and any and all other applications for which a foreign or domestic priority claim is identified in the Application Data Sheet as filed with the present application, are hereby incorporated by reference under 37 CFR 1.57.

### TECHNICAL FIELD

The present disclosure relates to wearable pulse oximetry systems for measuring and/or monitoring a subject's physiological information.

### BACKGROUND

Hospitals, nursing homes, and other patient care facilities typically include patient monitoring devices at one or more bedsides in the facility. Patient monitoring devices generally include sensors, processing equipment, and displays for obtaining and analyzing a medical subject's physiological parameters such as blood oxygen saturation level, respiratory rate, pulse, and other parameters, such as those monitored on commercially available patient monitors from Masimo Corporation of Irvine, California. Clinicians, including doctors, nurses, and other medical personnel, use the physiological parameters and trends of those parameters obtained from patient monitors to diagnose illnesses and to prescribe treatments. Clinicians also use the physiological parameters to monitor patients during various clinical situations to determine whether to increase the level of medical care given to patients.

### SUMMARY

Pulse oximetry is a widely accepted noninvasive procedure for measuring the oxygen saturation level of arterial blood, an indicator of a person's oxygen supply. Pulse oximetry sensors generally include one or more light sources transmitting optical radiation into or reflecting off through a portion of the body. After attenuation by tissue and fluids of the portion of the body, one or more photodetection devices detect the attenuated light and output one or more detector signals responsive to the detected attenuated light. The pulse oximetry sensor can be utilized for determination of a variety of physiological parameters and/or characteristics, including but not limited to oxygen saturation (SpO₂), pulse rate, a plethysmograph waveform, perfusion index (PI), pleth variability index (PVI), methemoglobin (MetHb), carboxyhemoglobin (CoHb), total hemoglobin (tHb), glucose, and/or otherwise, and the pulse oximetry sensor can be utilized for display on one or more monitors the foregoing parameters individually, in groups, in trends, as combinations, or as an overall wellness or other index.

In some circumstances, it can be advantageous to select a finger, such as a pinky finger, as a site for pulse oximetry. In some circumstances, it can be advantageous to select a wrist as a site for pulse oximetry alone or in combination with utilization of a finger site for pulse oximetry. The present disclosure describes various implementations for physiological sensor systems which secure to a subject (for example, to a wrist and/or finger of the subject) and employ pulse oximetry at a finger and/or wrist of the subject. Various implementations disclosed herein provide increased user comfort, increased user ergonomics, facilitate better sensor-skin contact and engagement in order to provide more accurate physiological parameter determination, and provide better stability in securement. Some implementations of the sensor systems disclosed herein: include a finger portion that secures to a subject's pinky finger and operably positions emitter(s) and detector(s); a wrist portion configured to be secured to the subject's wrist; and a cable that provides electrical communication between such finger portion and wrist portion. Some implementations of the sensor systems disclosed herein include a wrist portion configured to be secured to the subject's wrist and operably position emitter(s) and detector(s). Some implementations of the sensor systems disclosed herein include one or more cable retainers configured to facilitate positioning and/or securement of such cable along an outer side/edge of a subject's hand/palm and pinky finger, which can advantageously provide increased mobility to the subject's wrist, hand, and pinky finger without any or with little pulling and/or binding of the cable during movement as would occur if the cable were routed/positioned along a top or bottom side of the subject's hand/palm and/or pinky finger (e.g., due to a change in overall cable path length between bending the pinky finger closed and/or bending the hand at the wrist and the non-bent/non-closed position(s)). Additionally, such positioning and/or securement of the cable along the outer side/edge of the subject's hand/palm and pinky finger can advantageously increase comfort and tolerability to the subject. Some implementations of the sensor systems disclosed herein provide for a sensor system that can easily be adapted and/or customized to fit subjects with body parts (e.g., wrists, hands, and fingers) of various sizes and/or shapes. For example, the cable can be routed and/or positioned such that it is directed generally away from the hand/palm of the subject as it extends from the wrist portion of the sensor system and then bent back towards the hand/palm of the subject to connect to the finger portion of the sensor system, which can allow for a subject to customize the length of the cable that extends along their hand/palm and finger by adjusting the size of the loop that is created by such bend and such positioning/routing (e.g., so that the sensor system can be customized to the subject's hand/palm and finger length). Further, in some implementations, the finger portion and/or the wrist portion of the sensor systems described herein can be resilient and flexible such that it can adapt to pinky fingers and/or wrist sizes of various sizes/shapes. Additionally, the finger portion and/or wrist portion of the sensor systems described herein can be provided in various sizes/shapes (e.g., small, medium, large) to further enable a customized fit for the subject. For example, a plurality of ring members (each having different sizes) can be utilized to provide accommodation for different finger sizes of different subjects. Some implementations of the sensor systems disclosed herein can advantageously provide for a sensor system that is reusable and/or durable (e.g., lasting weeks and/or months).

Disclosed herein is a pulse oximetry system configured to secure to a wrist and measure a blood oxygen saturation at a tissue site on a pinky finger of a subject, the pulse oximetry system comprising: a sensor dock assembly; a first cable retainer; a second cable retainer; and a sensor hub. The sensor dock assembly comprises: a dock and a strap configured to secure the dock to the subject's wrist; a ring member configured to secure around a first portion of the subject's pinky finger; one or more emitters operably positioned by a first portion of the ring member, the one or more emitters configured to emit optical radiation into tissue of the subject's pinky finger; one or more detectors operably positioned by a second portion of the ring member that is opposite the first portion of the ring member, the one or more detectors configured to detect at least a portion of the optical radiation emitted from the one or more emitters after passing through said tissue and output at least one signal responsive to the detected optical radiation; and a cable connected to and extending from the dock and in electrical communication with the one or more emitters and the one or more detectors, the cable configured to transmit said at least one signal outputted by the one or more detectors to the dock. The first cable retainer is disposed on the strap or the dock and comprises a prong configured to secure a first portion of the cable. The second cable retainer is configured to secure around a second portion of the subject's pinky finger between the ring member and the subject's palm and comprises a prong configured to secure a second portion of the cable. The first and second cable retainers are configured to allow the cable to be positioned along an outside edge of a palm of the subject and along at least a portion of an outside edge of the subject's pinky finger when the ring member is secured to the first portion of the subject's pinky finger and the dock is secured to the subject's wrist. The sensor hub is configured to releasably connect to the dock and comprises one or more hardware processors and a battery. When the sensor hub is connected to the dock of the sensor dock assembly: the sensor dock assembly is configured to receive power from the battery of the sensor hub; and the one or more hardware processors of the sensor hub are configured to receive and process said at least one signal outputted by the one or more detectors of the sensor dock assembly to determine at least one physiological parameter of the subject.

In the above pulse oximetry system or in other implementations as described herein, one or more of the following features can also be provided. In some implementations, the ring member is configured to secure around a nail bed of the subject's pinky finger. In some implementations, the ring member comprises a resilient and flexible material. In some implementations, the dock comprises a base and two arms extending from the base and separated from one another by a gap sized to receive the sensor hub, said arms configured to releasably connect to sides of the sensor hub, and wherein said cable is connected to and extends from said base. In some implementations, the arms of the dock comprise one or more retaining features that are configured to engage one or more corresponding retaining features on the sides of the sensor hub. In some implementations, the one or more retaining features of the arms of the dock comprise one or more protrusions extending from inward facing surfaces of said arms; and the one or more corresponding retaining features on the sides of the sensor hub comprise one or more recesses configured to receive said one or more protrusions. In some implementations, the dock comprises an electrical connector in electrical communication with the cable that engages an electrical connector of the sensor hub when the sensor hub is connected to the dock. In some implementations, the arms of the dock each comprise one or more strap loops configured to be coupled with the strap. In some implementations, when the sensor dock assembly is secured to the subject's wrist and the ring member is secured to said pinky finger, the cable extends from the dock in a direction away from a hand of the subject and is bent towards the subject's pinky finger to connect to the one or more emitters and the one or more detectors. In some implementations, the cable comprises a first end connected to the dock and a second end opposite the first end; and the cable is split into a first portion and a second portion at the second end, said first portion connected to the one or more emitters and said second portion connected to the one or more detectors. In some implementations, when the ring member is secured to the subject's pinky finger: the first portion of the cable extends around said outside edge and/or extends along a portion of a top of the subject's pinky finger; and the second portion of the cable extends around said outside edge and/or extends along a portion of a bottom of the subject's pinky finger. In some implementations, the one or more emitters are disposed within an emitter housing and the one or more detectors are disposed within a detector housing. In some implementations, the ring member comprises a body comprising: an opening configured to receive a portion of the subject's pinky finger therethrough; a first cavity sized and shaped to receive the emitter housing; and a second cavity sized and shaped to receive the detector housing. In some implementations, the body of the ring member comprises a first notch configured to receive the first portion of the cable at the second end and a second notch configured to receive the second portion of the cable at the second end, wherein said first notch is positioned adjacent the first cavity and said second notch is positioned adjacent the second cavity. In some implementations, the ring member further comprises: a first cavity opening extending through a portion of the body into the first cavity, said first cavity opening sized and shaped to correspond with a size and a shape of a first protrusion that extends from the emitter housing; and a second cavity opening extending through a portion of the body into the second cavity, said second cavity opening sized and shaped to correspond with a size and a shape of a second protrusion that extends from the detector housing. In some implementations, when the emitter housing is disposed within the first cavity of the ring member, the first protrusion of the emitter housing is positioned within the first cavity opening; and when the detector housing is disposed within the second cavity of the ring member, the second protrusion of the detector housing is positioned within the second cavity opening. In some implementations, said first protrusion is at least one of shaped and sized different than said second protrusion. In some implementations, the pulse oximetry system further comprises an optical transmission material configured to be positioned between the one or more emitters and said tissue when the ring member is secured to the subject's pinky finger. In some implementations, the optical transmission material is configured to focus or diffuse optical radiation emitted from said one or more emitters. In some implementations, the optical transmission material comprises a lens or a diffuser. In some implementations, the pulse oximetry system further comprises an optical transmission material configured to be positioned between the one or more detectors and said tissue when the ring member is secured to the subject's pinky finger. In some implementations, the optical transmission material is configured to direct optical radiation toward the one or more detectors after passing through said tissue. In some implementations, the optical transmission material comprises a lens. In some implementations, the sensor dock assembly does not comprise a battery. In some implementations, the sensor dock assembly does not comprise a processor. In some implementations, the sensor dock assembly does not comprise a communication module. In some implementations, the sensor dock assembly is configured to transition from a non-operational mode when the sensor hub is disconnected from the dock to an operational mode when sensor hub is connected to the dock. In some implementations, in the operational mode, the pulse oximetry system is configured to determine at least one physiological parameter of the subject. In some implementations, the dock comprises an RFID tag configured to communicate with an RFID reader of the sensor hub. In some implementations, the sensor hub comprises a communication module configured to wirelessly communicate with separate devices. In some implementations, the sensor hub comprises a vibration motor in electrical communication with said one or more hardware processors, and said one or more hardware processors are configured to instruct said vibration motor to cause the sensor hub to vibrate. In some implementations, said one or more hardware processors are configured to: compare said determined at least one physiological parameter to one or more thresholds; and instruct said vibration motor to cause the sensor hub to vibrate based on said comparison of said determined at least one physiological parameter to said one or more thresholds. In some implementations, said second cable retainer comprises a ring configured to secure around the second portion of the subject's pinky finger between the ring member and the subject's palm.

Disclosed herein is a pulse oximetry system configured to secure to a wrist and measure a blood oxygen saturation at a finger of a subject, the pulse oximetry system comprising: a sensor dock assembly and a sensor hub. The sensor dock assembly comprises: a dock configured to be secured to the subject's wrist; a ring member configured to secure around the subject's finger; one or more emitters operably positioned by a first portion of the ring member, the one or more emitters configured to emit optical radiation into tissue of the subject's finger; one or more detectors operably positioned by a second portion of the ring member, the one or more detectors configured to detect at least a portion of the optical radiation emitted from the one or more emitters after passing through said tissue and output at least one signal responsive to the detected optical radiation; and a cable connected to and extending from the dock and in electrical communication with the one or more emitters and the one or more detectors, the cable configured to transmit said at least one signal outputted by the one or more detectors to the dock. The sensor hub is configured to releasably connect to the dock and comprises one or more hardware processors and a battery. When the sensor hub is connected to the dock of the sensor dock assembly: the sensor dock assembly is configured to receive power from the battery of the sensor hub; and the one or more hardware processors of the sensor hub are configured to receive and process said at least one signal outputted by the one or more detectors of the sensor dock assembly to determine at least one physiological parameter of the subject.

In the above pulse oximetry system or in other implementations as described herein, one or more of the following features can also be provided. In some implementations, the subject's finger comprises a pinky finger. In some implementations, the ring member is configured to secure around a nail bed of the subject's finger. In some implementations, the ring member comprises a resilient and flexible material. In some implementations, the second portion of the ring member is opposite the first portion of the ring member. In some implementations, the dock comprises a base and two arms extending from the base and separated from one another by a gap sized to receive the sensor hub, said arms configured to releasably connect to sides of the sensor hub, and wherein said cable is connected to and extends from said base. In some implementations, the arms of the dock comprise one or more retaining features that are configured to engage one or more corresponding retaining features on the sides of the sensor hub. In some implementations, the one or more retaining features of the arms of the dock comprise one or more protrusions extending from inward facing surfaces of said arms; and the one or more corresponding retaining features on the sides of the sensor hub comprise one or more recesses configured to receive said one or more protrusions. In some implementations, the dock comprises an electrical connector in electrical communication with the cable that engages an electrical connector of the sensor hub when the sensor hub is connected to the dock. In some implementations, the arms of the dock each comprise one or more strap loops configured to be coupled with a strap for securing the dock to the subject's wrist. In some implementations, the pulse oximetry system further comprises a strap configured to secure the dock to the subject's wrist. In some implementations, when the sensor dock assembly is secured to the subject's wrist and the ring member is secured to said pinky finger, the cable extends from the dock in a direction away from a hand of the subject and is bent towards the subject's pinky finger to connect to the one or more emitters and the one or more detectors. In some implementations, the cable is positioned along an outside edge of a palm of the subject and along at least a portion of an outside edge of the subject's pinky finger. In some implementations, the cable is retained in said position by one or more cable retainers configured to secure one or more portions of the cable. In some implementations, disclosed is a system including the pulse oximetry system and one or more cable retainers configured to secure one or more portions of the cable and retain the cable in said position. In some implementations, at least one of said cable retainers is disposed on the dock or on a strap configured to secure the dock to the subject's wrist and comprises a prong configured to secure said one or more portions of the cable. In some implementations, at least one of said cable retainers comprises: a ring configured to secure around the subject's pinky finger between the ring member and the subject's palm; and a prong configured to secure said one or more portions of the cable. In some implementations, the cable comprises a first end connected to the dock and a second end opposite the first end; and the cable is split into a first portion and a second portion at the second end, said first portion connected to the one or more emitters and said second portion connected to the one or more detectors. In some implementations, when the ring member is secured to the subject's pinky finger: the first portion of the cable extends around said outside edge and/or extends along a portion of a top of the subject's pinky finger; and the second portion of the cable extends around said outside edge and/or extends along a portion of a bottom of the subject's pinky finger. In some implementations, the one or more emitters are disposed within an emitter housing and the one or more detectors are disposed within a detector housing. In some implementations, the ring member comprises a body comprising: an opening configured to receive a portion of the subject's finger therethrough; a first cavity sized and shaped to receive the emitter housing; and a second cavity sized and shaped to receive the detector housing. In some implementations, the body of the ring member comprises a first notch configured to receive a portion of the cable in electrical communication with the one or more emitters and a second notch configured to receive a portion of the cable in electrical communication with the one or more detectors, wherein said first notch is positioned adjacent the first cavity and said second notch is positioned adjacent the second cavity. In some implementations, the body of the ring member comprises: a first cavity opening extending through a portion of the body into the first cavity, said first cavity opening sized and shaped to correspond with a size and a shape of a first protrusion that extends from the emitter housing; and a second cavity opening extending through a portion of the body into the second cavity, said second cavity opening sized and shaped to correspond with a size and a shape of a second protrusion that extends from the detector housing. In some implementations, when the emitter housing is disposed within the first cavity of the ring member, the first protrusion of the emitter housing is positioned within the first cavity opening; and when the detector housing is disposed within the second cavity of the ring member, the second protrusion of the detector housing is positioned within the second cavity opening. In some implementations, said first protrusion is shaped and sized different than said second protrusion. In some implementations, the one or more detectors are at least partially enclosed by a detector shield configured to prevent optical radiation that has not passed through said tissue from reaching the one or more detectors. In some implementations, the pulse oximetry system further comprises an optical transmission material configured to be positioned between the one or more emitters and said tissue when the ring member is secured to the subject's finger. In some implementations, the optical transmission material is configured to focus or diffuse optical radiation emitted from said one or more emitters. In some implementations, the optical transmission material comprises a lens or a diffuser. In some implementations, the pulse oximetry system further comprises an optical transmission material configured to be positioned between the one or more detectors and said tissue when the ring member is secured to the subject's finger. In some implementations, the optical transmission material is configured to direct optical radiation towards the one or more detectors after passing through said tissue. In some implementations, the optical transmission material comprises a lens. In some implementations, the sensor dock assembly does not comprise a battery. In some implementations, the sensor dock assembly does not comprise a processor. In some implementations, the sensor dock assembly does not comprise a communication module. In some implementations, the sensor dock assembly is configured to transition from a non-operational mode when the sensor hub is disconnected from the dock to an operational mode when sensor hub is connected to the dock. In some implementations, in the operational mode, the pulse oximetry system is configured to determine at least one physiological parameter of the subject. In some implementations, the sensor dock assembly comprises an RFID tag configured to communicate with an RFID reader of the sensor hub. In some implementations, the sensor hub comprises a communication module configured to wirelessly communicate with separate devices. In some implementations, the sensor hub comprises a vibration motor in electrical communication with said one or more hardware processors, and said one or more hardware processors are configured to instruct said vibration motor to cause the sensor hub to vibrate. In some implementations, said one or more hardware processors are configured to: compare said determined at least one physiological parameter to one or more thresholds; and instruct said vibration motor to cause the sensor hub to vibrate based on said comparison of said determined at least one physiological parameter to said one or more thresholds.

Disclosed herein is a pulse oximetry system comprising: a finger portion, a wrist portion, a cable, a strap, and a cable retainer. The finger portion is configured to secure to a finger of a subject, the finger portion comprising at least one emitter configured to emit optical radiation into tissue of the subject's finger and at least one detector configured to detect at least a portion of the optical radiation after passing through said tissue, the at least one detector further configured to output at least one signal responsive to the detected optical radiation. The wrist portion is configured for placement on a wrist of the subject, the wrist portion comprising one or more hardware processors. The cable electrically connects the finger portion and the wrist portion together, the cable configured to transmit said at least one signal outputted by the at least one detector to the wrist portion, the one or more hardware processors configured to receive and process said at least one signal to determine at least one physiological parameter of the subject. The strap is configured to be coupled to the wrist portion and secure the wrist portion to the subject's wrist when in use. The cable retainer is connected to the strap and configured to be positioned on a side of the subject's wrist when the strap is in use, the cable retainer configured to secure a portion of the cable and allow the cable to extend along a side of the subject's palm and along at least a portion of the subject's finger.

In the above pulse oximetry system or in other implementations as described herein, one or more of the following features can also be provided. In some implementations, the finger portion comprises a ring member configured to secure around a portion of the subject's finger, an emitter housing that at least partially encloses said at least one emitter, and a detector housing that at least partially encloses said at least one detector, and wherein the ring member is configured to operably position the emitter and detector housings. In some implementations, the cable retainer is integrally formed with the strap. In some implementations, the wrist portion comprises a sensor hub and a dock, the sensor hub comprising said one or more hardware processors, and the dock comprising a base and two arms extending from the base and separated from one another by a gap sized to receive the sensor hub, said arms configured to releasably slidably connect to sides of the sensor hub. In some implementations, the cable comprises a first end connected to the wrist portion and a second end opposite the first end, and the cable is split into a first portion and a second portion at the second end, said first portion connected to the at least one emitter and said second portion connected to the at least one detector.

Disclosed herein is a pulse oximetry system comprising: a finger portion, a wrist portion, a cable, a strap, and a cable retainer. The finger portion is configured to secure to a finger of a subject, the finger portion comprising at least one emitter configured to emit optical radiation into tissue of the subject's finger and at least one detector configured to detect at least a portion of the optical radiation after passing through said tissue, the at least one detector further configured to output at least one signal responsive to the detected optical radiation. The wrist portion is configured for placement on a wrist of the subject, the wrist portion comprising one or more hardware processors. The cable electrically connects the finger portion and the wrist portion together, the cable configured to transmit said at least one signal outputted by the at least one detector to the wrist portion, the one or more hardware processors configured to receive and process said at least one signal to determine at least one physiological parameter of the subject. The strap is configured to be coupled to the wrist portion and secure the wrist portion to the subject's wrist when in use. The cable retainer is connected to the strap and configured to secure a portion of the cable. The wrist portion does not include any cable retainers for securing the cable.

In the above pulse oximetry system or in other implementations as described herein, one or more of the following features can also be provided. In some implementations, the finger portion comprises a ring member configured to secure around a portion of the subject's finger, an emitter housing that at least partially encloses said at least one emitter, and a detector housing that at least partially encloses said at least one detector, and the ring member is configured to operably position the emitter and detector housings. In some implementations, the wrist portion comprises a sensor hub and a dock, the sensor hub comprising said one or more hardware processors, and the dock comprises a base and two arms extending from the base and separated from one another by a gap sized to receive the sensor hub, said arms configured to releasably slidably connect to sides of the sensor hub.

Disclosed herein is a pulse oximetry system configured to secure to a wrist and a finger of a subject, the pulse oximetry system comprising: a dock, an emitter housing, a detector housing, a cable, a ring member, and a sensor hub. The dock is configured to be secured to the subject's wrist. The emitter housing comprises at least one emitter configured to emit optical radiation into tissue of the subject's finger. The detector housing comprises at least one detector configured to detect at least a portion of the optical radiation after passing through said tissue, the at least one detector further configured to output at least one signal responsive to the detected optical radiation. The cable is connected to and extends between the dock and the emitter and detector housings, the cable configured to transmit said at least one signal outputted by the at least one detector to the dock. The ring member is configured to secure around the subject's finger and operably position the emitter and detector housings adjacent the finger, wherein the ring member is configured to removably connect to the emitter and detector housings. The sensor hub is configured to releasably connect to the dock, the sensor hub comprising one or more hardware processors configured to receive and process said at least one signal to determine at least one physiological parameter of the subject.

In the above pulse oximetry system or in other implementations as described herein, one or more of the following features can also be provided. In some implementations, the pulse oximetry system further comprises one or more cable retainers configured to secure portions of the cable when the pulse oximetry system is in use. In some implementations, the cable comprises a first end connected to the dock and a second end opposite the first end, and the cable is split into a first portion and a second portion at the second end, said first portion connected to the at least one emitter and said second portion connected to the at least one detector.

Disclosed herein is pulse oximetry system comprising: a finger portion, a wrist portion, and a cable. The finger portion is configured to secure to a finger of a subject, the finger portion comprising at least one emitter configured to emit optical radiation into tissue of the subject's finger and at least one detector configured to detect at least a portion of the optical radiation after passing through said tissue, the at least one detector further configured to output at least one signal responsive to the detected optical radiation. The wrist portion is configured for placement on a wrist of the subject, the wrist portion comprising one or more hardware processors. The cable electrically connects the finger portion and the wrist portion together, the cable configured to transmit said at least one signal outputted by the at least one detector to the wrist portion, the one or more hardware processors configured to receive and process said at least one signal to determine at least one physiological parameter of the subject, the cable comprising a first end connected to the wrist portion and a second end opposite the first end, the cable comprising a split into a first portion and a second portion at said second end, said first portion connecting to the at least one emitter and said second portion connecting to the at least one detector.

In the above pulse oximetry system or in other implementations as described herein, one or more of the following features can also be provided. In some implementations, when the finger portion is secured to the subject's pinky finger, said first portion and said second portion of the cable straddle a top and a bottom, respectively, of the subject's finger. In some implementations, the pulse oximetry system further comprises at least one strap configured to secure the wrist portion to the subject's wrist, wherein the wrist portion comprises a first component and a second component configured to removably secure to one another, the second component coupled with the at least one strap, and wherein the first component comprises said one or more hardware processors and a battery. In some implementations, the pulse oximetry system further comprises one or more cable retainers configured to secure portions of the cable when the pulse oximetry system is in use. In some implementations, at least one of said one or more cable retainers is configured to permanently or removably attached to a portion of the at least one strap.

Disclosed herein is a pulse oximetry system comprising: a finger portion, a wrist portion, and a cable. The finger portion is configured to secure to a finger of a subject, the finger portion comprising at least one emitter configured to emit optical radiation into tissue of the subject's finger and at least one detector configured to detect at least a portion of the optical radiation after passing through said tissue, the at least one detector further configured to output at least one signal responsive to the detected optical radiation. The wrist portion is configured for placement on a wrist of the subject, the wrist portion comprising one or more hardware processors. The cable electrically connects the finger portion and the wrist portion together, the cable configured to transmit said at least one signal outputted by the at least one detector to the wrist portion, the one or more hardware processors configured to receive and process said at least one signal to determine at least one physiological parameter of the subject.

In the above pulse oximetry system or in other implementations as described herein, one or more of the following features can also be provided. In some implementations, said cable extends from a corner of the wrist portion. In some implementations, the finger portion comprises a ring member configured to secure around a finger of the user, and wherein the ring member is configured to operably position the at least one emitter and the at least one detector relative to the finger when secured thereto. In some implementations, said cable is configured to removably connect to the wrist portion. In some implementations, said cable is configured to permanently connect to the wrist portion. In some implementations, the wrist portion comprises a watch. In some implementations, the pulse oximetry system further comprises at least one strap configured to secure the wrist portion to the subject's wrist, wherein the wrist portion comprises a first component and a second component configured to removably secure to one another, the second component coupled with the at least one strap, and wherein the first component comprises said one or more hardware processors and a battery. In some implementations, said cable extends from a corner of the second component. In some implementations, the second component does not comprise a battery. In some implementations, the first component does not include a display. In some implementations, the first component is configured to receive one or more signals outputted by the at least one detector via the cable and determine one or more physiological parameters based on said one or more signals. In some implementations, the first component is further configured to wirelessly transmit said one or more physiological parameters to a separate device.

Disclosed herein is a pulse oximetry system comprising: a ring member configured to secure around a finger of a subject; an emitter housing configured to be operably positioned by a first portion of the ring member, the emitter housing comprising at least one emitter configured to emit optical radiation into tissue of the subject's finger; a detector housing configured to be operably positioned by a second portion of the ring member, the detector housing comprising at least one detector configured to detect at least a portion of the optical radiation after passing through said tissue, the at least one detector further configured to output at least one signal responsive to the detected optical radiation; and a cable having a first end and a second end opposite the first end, wherein the cable is connected to the emitter and detector housings at the second end.

In the above pulse oximetry system or in other implementations as described herein, one or more of the following features can also be provided. In some implementations, said first and second portions of the ring member are opposite one another such that, when the emitter and detector housings are respectively positioned by the first and second portions of the ring member and the ring member is secured around the finger, the at least one emitter and the at least one detector are substantially aligned with one another. In some implementations, the ring member includes a first cavity sized and shaped to retain the emitter housing and a second cavity sized and shaped to retain the detector housing. In some implementations, the first end of the cable is configured to removably connect a wearable device (for example, an electronic device such as a hub and/or a wrist portion as described herein), said wearable device configured to be secured to a wrist of the subject. In some implementations, the first end of the cable is configured to transmit said at least one signal outputted by the at least one detector to the wearable device, and wherein one or more hardware processors of the wearable device are configured to receive and process said at least one signal to determine at least one physiological parameter of the subject. In some implementations, the wearable device comprises a watch. In some implementations, the wearable device comprises at least one strap, a first component comprises a battery and said one or more hardware processors, and a second component coupled to the at least one strap and configure to removably connect to the first component. In some implementations, the first end of the cable is permanently secured to the wearable device. In some implementations, the first end of the cable is removably connectable to the wearable device. In some implementations, the cable splits into a first portion and a second portion at the second end, and wherein the first portion connects to the emitter housing and wherein the second portion connects to the detector housing.

Disclosed herein is a pulse oximetry system comprising: a wrist portion configured for placement on a wrist of a subject, the wrist portion comprising a first component and a second component configured to removably secure to one another, the first component comprising one or more hardware processors and a battery, the second component comprising at least one emitter configured to emit optical radiation into tissue of the subject's wrist and at least one detector configured to detect at least a portion of the optical radiation reflected from said tissue, the at least one detector further configured to output at least one signal responsive to the detected optical radiation; and at least one strap configured to secure the wrist portion to the subject's wrist; wherein the one or more hardware processors of the first component are configured to receive and process said at least one signal to determine at least one physiological parameter of the subject when the first component and the second component are secured to one another.

In the above pulse oximetry system or in other implementations as described herein, one or more of the following features can also be provided. In some implementations, the first component is further configured to wirelessly transmit said one or more physiological parameters to a separate device. In some implementations, the first component does not comprise a display. In some implementations, said at least one strap is coupled to the second component. In some implementations, the pulse oximetry system further comprises: a finger portion configured to secure to a finger of the subject, the finger portion comprising at least one emitter configured to emit optical radiation into tissue of the subject's finger and at least one detector configured to detect at least a portion of the optical radiation after passing through the tissue of the subject's finger, the at least one detector of the finger portion further configured to output at least one signal responsive to the detected optical radiation; and a cable electrically connecting the finger portion and the wrist portion together, the cable configured to transmit said at least one signal outputted by the at least one detector of the finger portion to the wrist portion; wherein the one or more hardware processors of the first component are configured to receive and process said at least one signal from the at least one detector of the finger portion to determine at least one physiological parameter of the subject when the first component and the second component are secured to one another.

Disclosed herein is a pulse oximetry system comprising: a wrist portion configured for placement on a wrist of a subject, the wrist portion comprising a first component and a second component configured to removably secure to one another, the first component comprising one or more hardware processors, a battery, a communication module, at least one ECG electrode configured to measure an ECG of the subject and output at least one signal responsive to the measured ECG, at least one emitter configured to emit optical radiation into tissue of the subject's wrist, and at least one detector configured to detect at least a portion of the optical radiation reflected from said tissue, the at least one detector further configured to output at least one signal responsive to the detected optical radiation; and at least one strap coupled to the second component and configured to secure the wrist portion to the subject's wrist; wherein the one or more hardware processors of the first component are configured to receive and process said at least one signal from the at least one ECG electrode and said at least one signal from the at least one detector to determine at least one physiological parameter of the subject when the first component and the second component are secured to one another; and wherein the communication module of the first component is configured to wirelessly transmit said at least one physiological parameter to a separate device.

In the above pulse oximetry system or in other implementations as described herein, one or more of the following features can also be provided. In some implementations, the first component does not comprise a display. In some implementations, the second component does not comprise a hardware processor, a battery, a memory, or a sensor. In some implementations, the first component secures to the second component by a snap fit by being pressed into the second component from below. In some implementations, the first component is released from the second component by being pressed down from above relative to the second component. In some implementations, the first component does not electrically communicate with the second component. In some implementations, the pulse oximetry system further comprises: a finger portion configured to secure to a finger of the subject, the finger portion comprising at least one emitter configured to emit optical radiation into tissue of the subject's finger and at least one detector configured to detect at least a portion of the optical radiation after passing through the tissue of the subject's finger, the at least one detector of the finger portion further configured to output at least one signal responsive to the detected optical radiation; and a cable electrically connecting the finger portion and the first component of the wrist portion together, the cable configured to transmit said at least one signal outputted by the at least one detector of the finger portion to the first component of the wrist portion; wherein the one or more hardware processors of the first component are configured to receive and process said at least one signal from the at least one detector of the finger portion to determine at least one physiological parameter of the subject when the first component and the second component are secured to one another.

Disclosed herein is a pulse oximetry system comprising: a wrist portion configured for placement on a wrist of a subject, the wrist portion comprising one or more hardware processors; an emitter housing comprising at least one emitter configured to emit optical radiation into tissue of a finger of the subject; a detector housing comprising at least one detector configured to detect at least a portion of the optical radiation after passing through said tissue, the at least one detector further configured to output at least one signal responsive to the detected optical radiation; a finger portion comprising a first cavity configured to removably receive the emitter housing and a second cavity configured to removably receive the detector housing, said first and second cavities being spaced from one another, wherein the finger portion is configured to secure to the subject's finger and operably position the emitter and detector housings adjacent portions of the subject's finger; and a cable connecting the wrist portion to the emitter and detector housings, the cable configured to transmit said at least one signal outputted by the at least one detector to the wrist portion, the one or more hardware processors configured to receive and process said at least one signal to determine at least one physiological parameter of the subject.

In the above pulse oximetry system or in other implementations as described herein, one or more of the following features can also be provided. In some implementations, the cable comprises a first end connected to the wrist portion and a second end opposite the first end, wherein the cable splits into a first portion and a second portion at said second end, said first portion of the cable connecting to the at least one emitter and said second portion of the cable connecting to the at least one detector. In some implementations, said finger is a pinky finger of the subject, and wherein when the finger portion is secured to said pinky finger, said first portion and said second portion of the cable straddle a top and a bottom, respectively, of the subject's pinky finger. In some implementations, the pulse oximetry system further comprises a cable retainer configured to secure around a portion of the subject's finger between the finger portion and a palm of the subject, the cable retainer comprising a prong configured to secure a portion of the cable. In some implementations, the first and second cavities of the finger portion are at generally opposite portions of the finger portion such that, when the emitter and detector housings are respectively positioned by the first and second cavities, the at least one emitter and the at least one detector are substantially aligned with one another. In some implementations, the finger portion comprises a resilient and flexible material. In some implementations, said cable does not extend from a side of the wrist portion that faces towards a hand of the subject when the wrist portion is placed on the wrist of the subject. In some implementations, said cable extends from a corner of the wrist portion that faces away from a hand of the subject when the wrist portion is placed on the wrist of the subject.

Disclosed herein is a pulse oximetry system comprising: a wrist portion configured for placement on a wrist of a subject, the wrist portion comprising one or more hardware processors; an emitter housing comprising at least one emitter configured to emit optical radiation into tissue of a finger of the subject; a detector housing comprising at least one detector configured to detect at least a portion of the optical radiation after passing through said tissue, the at least one detector further configured to output at least one signal responsive to the detected optical radiation; a cable connecting the wrist portion to the emitter and detector housings, the cable configured to transmit said at least one signal outputted by the at least one detector to the wrist portion, the one or more hardware processors configured to receive and process said at least one signal to determine at least one physiological parameter of the subject; a first ring member configured to secure to the subject's finger, the first ring member further configured to removably receive the emitter and detector housings and operably position the emitter and detector housings adjacent portions of the subject's finger; and a second ring member configured to secure to the subject's finger, the second ring member further configured to removably receive the emitter and detector housings and operably position the emitter and detector housings adjacent portions of the subject's finger; wherein the second ring member has a different size than the first ring member.

In the above pulse oximetry system or in other implementations as described herein, one or more of the following features can also be provided. In some implementations, each of the first and second ring members comprises a resilient and flexible material. In some implementations, the first and second ring members comprise the same material. In some implementations, each of the first and second ring members comprises an opening configured to receive the subject's finger, and wherein the opening of the second ring member is smaller than the opening of the first ring member. In some implementations, the first ring member comprises a first cavity configured to removably receive the emitter housing and a second cavity configured to removably receive the detector housing; and the second ring member comprises a first cavity configured to removably receive the emitter housing and a second cavity configured to removably receive the detector housing. In some implementations, at least one of: the first cavity of the first ring member comprises a same size as the first cavity of the second ring member; and the second cavity of the first ring member comprises a same size as the second cavity of the second ring member. In some implementations, at least one of: said first and second cavities of the first ring member are spaced from one another; and said first and second cavities of the second ring member are spaced from one another. In some implementations, at least one of: said first and second cavities of the first ring member are substantially aligned with one another along a first axis that is perpendicular to a second axis that extends through a center of the first ring member; and said first and second cavities of the second ring member are substantially aligned with one another along a third axis that is perpendicular to a fourth axis that extends through a center of the second ring member.

For purposes of summarizing the disclosure, certain aspects, advantages and novel features of several implementations have been described herein. It is to be understood that not necessarily all such advantages are achieved in accordance with any particular implementation of the technology disclosed herein. Thus, the implementations disclosed herein can be implemented or carried out in a manner that achieves or optimizes one advantage or group of advantages as taught herein without necessarily achieving other advantages that can be taught or suggested herein.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIGS. 1A-1B illustrate perspective views of a pulse oximetry system secured to a subject's wrist and finger in accordance with aspects of this disclosure.

FIG. 1C illustrates the pulse oximetry system of FIG. 1A collecting and wirelessly transmitting physiological data to a computing device in accordance with aspects of this disclosure.

FIG. 2A illustrates a perspective view of the pulse oximetry system of FIG. 1A in accordance with aspects of this disclosure.

FIG. 2B illustrates a perspective view of the pulse oximetry system of FIG. 1A with a sensor hub detached from a dock of a sensor dock assembly in accordance with aspects of this disclosure.

FIG. 2C illustrates a schematic diagram of certain features of the pulse oximetry system of FIG. 1A in accordance with aspects of this disclosure.

FIG. 3 illustrates a perspective view of the sensor dock assembly of the pulse oximetry system of FIG. 1A with a ring member removed in accordance with aspects of this disclosure.

FIGS. 4A-4D illustrate perspective views of the dock of the sensor dock assembly of FIG. 3 in accordance with aspects of this disclosure.

FIGS. 5A-5B illustrate perspective views of portions of a cable of the sensor dock assembly, the ring member of the sensor dock assembly, and one or more emitters and one or more detectors of the sensor dock assembly of FIG. 3 in accordance with aspects of this disclosure.

FIGS. 5C-5D illustrate perspective views of portions of a cable of the sensor dock assembly and the ring member removed from one or more emitters and one or more detectors of the sensor dock assembly of FIG. 3 in accordance with aspects of this disclosure.

FIGS. 6A-6D illustrate perspective views of the ring member of the sensor dock assembly of FIG. 3 in accordance with aspects of this disclosure.

FIGS. 6E-6J illustrate a top, bottom, front, back, and side views, respectively, of the ring member of the sensor dock assembly of FIG. 3 in accordance with aspects of this disclosure.

FIGS. 7A-7B illustrate exploded perspective views of portions of the sensor dock assembly of FIG. 3 in accordance with aspects of this disclosure.

FIGS. 8A-8B illustrate perspective views of the sensor hub of the pulse oximetry system of FIG. 1A in accordance with aspects of this disclosure.

FIGS. 8C-8H illustrate top, bottom, side, front, and back views, respectively, of the sensor hub of FIGS. 8A-8B in accordance with aspects of this disclosure.

FIGS. 8I-8J illustrate exploded perspective views of the sensor hub of the pulse oximetry system of FIGS. 8A-8B in accordance with aspects of this disclosure.

FIGS. 9A-9B illustrate perspective views of a charging station in accordance with aspects of this disclosure.

FIGS. 9C-9H illustrate top, bottom, front, back, and side views, respectively, of the charging station of FIGS. 9A-9B in accordance with aspects of this disclosure.

FIGS. 9I-9J illustrate top perspective views of the charging station of FIGS. 9A-9B in accordance with aspects of this disclosure.

FIG. 9K illustrates a perspective cross-sectional view taken through the charging station of FIGS. 9A-9B in accordance with aspects of this disclosure.

FIG. 10 illustrates a perspective view of another implementation of a sensor dock assembly in accordance with aspects of this disclosure.

FIGS. 11A-11B illustrate perspective views of a clip member and a portion of a cable of the sensor dock assembly of FIG. 10 in accordance with aspects of this disclosure.

FIGS. 11C-11H illustrate top, bottom, front, back, and side views, respectively, of the clip member of FIGS. 11A-11B in accordance with aspects of this disclosure.

FIGS. 12A-12B illustrate perspective views of the clip member of the sensor dock assembly of FIGS. 11A-11B with a housing of the clip member separated from a biasing member of the clip member in accordance with aspects of this disclosure.

FIGS. 13A-13B illustrate perspective views of another implementation of a pulse oximetry system in the form of a watch secured to a subject's wrist and finger in accordance with aspects of this disclosure.

FIGS. 14A-14F illustrate another implementation of a pulse oximetry system in accordance with aspects of this disclosure.

FIGS. 14G-14H illustrate a pulse oximetry system that is a variant of the pulse oximetry system of FIGS. 14A-14F in accordance with aspects of this disclosure.

FIGS. 15A-15J illustrate another implementation of a pulse oximetry system in accordance with aspects of this disclosure.

FIG. 15K illustrates a pulse oximetry system that is a variant of the pulse oximetry system of FIGS. 15A-15J in accordance with aspects of this disclosure.

FIGS. 16A-16F illustrate a pulse oximetry system that is a variant of the pulse oximetry system of FIGS. 15A-15J in accordance with aspects of this disclosure.

FIGS. 17A-17G illustrate a pulse oximetry system that is a variant of the pulse oximetry systems of FIG. 15K and FIGS. 16A-16F in accordance with aspects of this disclosure.

FIGS. 18A-18M illustrate another implementation of a pulse oximetry system in accordance with aspects of this disclosure.

FIGS. 19A-19B illustrate a pulse oximetry system that is a variant of the pulse oximetry system of FIGS. 18A-18M in accordance with aspects of this disclosure.

FIGS. 20A-20B illustrate a pulse oximetry system that is a variant of the pulse oximetry system of FIGS. 19A-19B in accordance with aspects of this disclosure.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein. Furthermore, implementations disclosed herein can include several novel features, no single one of which is solely responsible for its desirable attributes or which is essential to practicing the systems, devices, and/or methods disclosed herein.

Disclosed herein are pulse oximetry systems that can be used to measure, monitor, transmit (for example, wirelessly or via wired connection), process, and/or determine one or more physiological parameters of a subject (which can also be referred to herein as a “user”, “patient”, or “wearer”). The disclosed pulse oximetry systems can generate one or more signals associated with and/or indicative of one or more physiological parameters of a subject and process such one or more signals to determine such physiological parameters. In some implementations, the disclosed pulse oximetry systems can generate and transmit one or more signals associated with and/or indicative of one or more physiological parameters of a subject to a separate monitoring, computing, and/or electrical device (for example, wirelessly or via wired connection), for example, a patient monitor, which is capable of processing and/or determining such physiological parameters based on the transmitted signals. Any of the disclosed pulse oximetry systems and/or devices in communication with the pulse oximetry systems can include hardware and/or software capable of determining and/or monitoring a variety of physiological parameters, including but not limited to blood oxygenation levels in veins and/or arteries, heart rate, blood flow, respiratory rates, and/or other physiological parameters or characteristics such as those discussed herein. Any of the pulse oximetry systems described herein can include and/or employ pulse oximetry (for example, via an optical sensor) to measure physiological parameters of the subject and/or to generate, transmit, and/or process one or more signals associated with and/or indicative of such physiological parameters and/or to determine such physiological parameters. As discussed below, such optical sensor can include one or more emitters configured to emit optical radiation (e.g., light) of one or more wavelengths (e.g., wavelength(s) in the visible spectrum, near infrared wavelength(s), infrared wavelength(s), far infrared wavelength(s), etc.) and one or more detectors configured to detect at least a portion of the emitted optical radiation after attenuation and/or after passing through tissue of the subject.

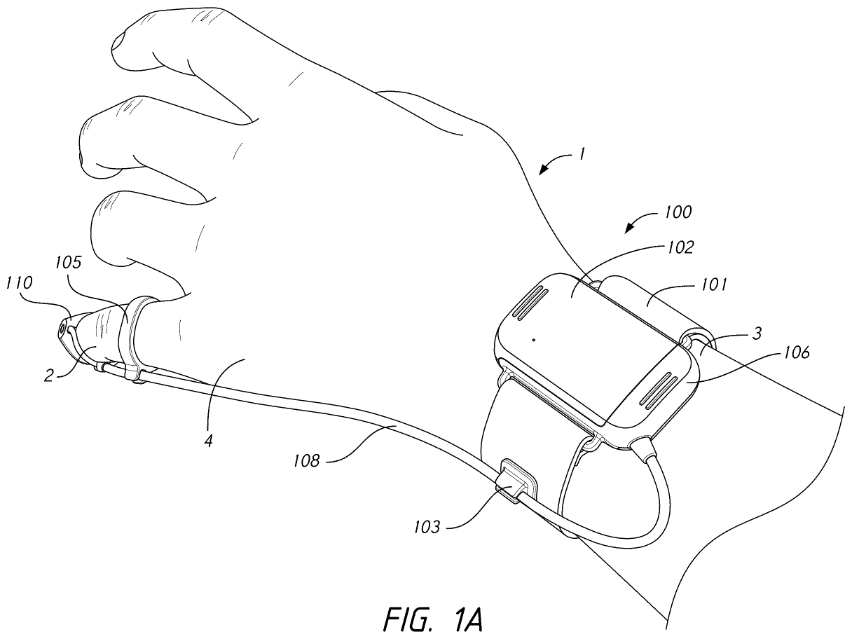

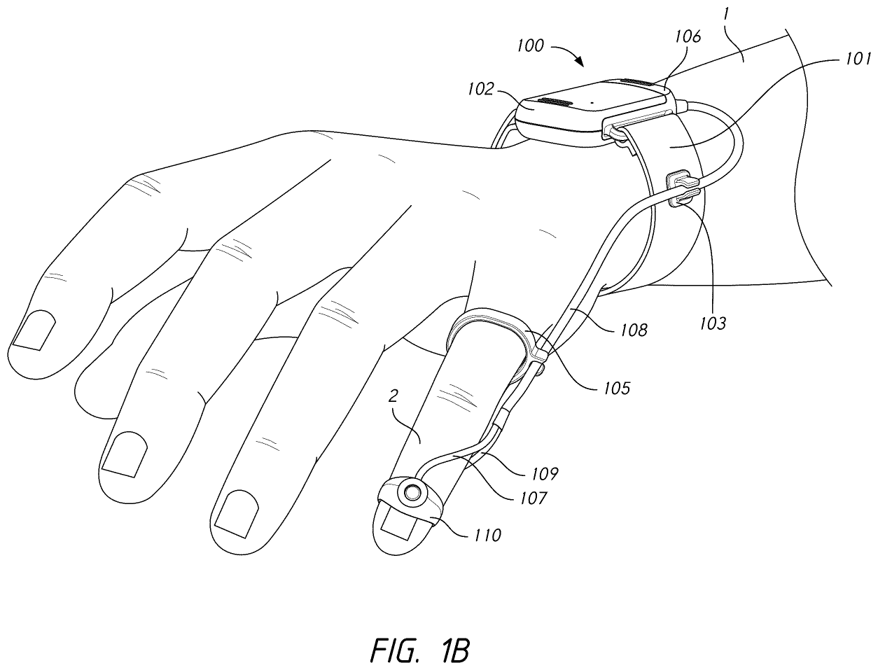

FIG. 1A illustrates a perspective view of a pulse oximetry system 100 (which can also be referred to herein as a “wearable sensor system” or “wearable physiological sensor system”) secured to a wrist 3 and a finger 2 of a subject 1. FIG. 1B illustrates another perspective view of the pulse oximetry system 100 of FIG. 1A secured to the subject's wrist 3 and finger 2. System 100 can include a finger portion that can be secured to a finger of a subject (such as a pinky finger) and a wrist portion that can be secured to a subject's wrist. System 100 can further include a cable that connects the finger and wrist portions of system 100. Such finger portion can comprise ring member 110 (and/or one or more emitters and one or more detectors and/or an emitter housing and detector housing such as any of those disclosed herein) described further below. Such wrist portion can comprise sensor hub 102 and/or dock 106 described further below. In some implementations, pulse oximetry system 100 includes a strap 101 and/or a cable 108, which are discussed further below. In some implementations, a cable retainer 103 and/or a cable retainer 105 can be utilized to secure the cable 108 to the subject 1 as shown and discussed further below.

Although FIGS. 1A-1B show the pulse oximetry system 100 secured to the wrist 3 and finger 2 of the subject 1 in a particular manner which can provide certain advantages as described herein, such illustrated manner and/or location of securement is not intended to be limiting. Pulse oximetry system 100 can be secured to various portions of the subject's wrist, hand, and/or finger(s) in a variety of manners and/or using a variety of methods. Alternatively, or in addition, in some implementations the pulse oximetry system 100 can be secured and/or placed adjacent to and/or around portions of a body of the subject 1 other than the wrist 3 and finger 2, such as adjacent to and/or around an ankle and a toe of the subject 1 among other regions of the subject's body. Accordingly, while pulse oximetry system 100 is described herein primarily with reference to a wrist, finger, and/or other portions of a hand of the subject 1, such description is not intended to be limiting, and the pulse oximetry system 100 can be utilized in connection with other portions of the subject's body. Furthermore, even though the pulse oximetry system 100 is shown secured to a left wrist and hand of the subject 1, such positioning is not intended to be limiting and the pulse oximetry system 100 can be secured to a right wrist and hand of the subject 1 in a similar way. FIG. 1C illustrates the pulse oximetry sensor of FIG. 1A secured to the subject 1 and wirelessly communicating with a separate computing device 10, which can be for example, a patient monitor (which can also be referred to herein as an “external patient monitor”), via any of a variety of wireless communication protocols (such as any of those discussed herein). The pulse oximetry system 100 can wirelessly transmit subject physiological data and/or physiological parameters to the separate computing device 10 as described further herein.

In some implementations, the pulse oximetry sensor(s) described herein (for example, pulse oximetry sensors 100 and 400) can be similar or identical to and/or incorporate any of the features described with respect to any of the devices, assemblies, and/or systems described and/or illustrated in U.S. Pat. Pub. No. US2020/0138288, filed Oct. 10, 2019, titled “SYSTEM FOR TRANSMISSION OF SENSOR DATA USING DUAL COMMUNICATION PROTOCOL,” which is hereby incorporated by reference in its entirety and for all purposes.

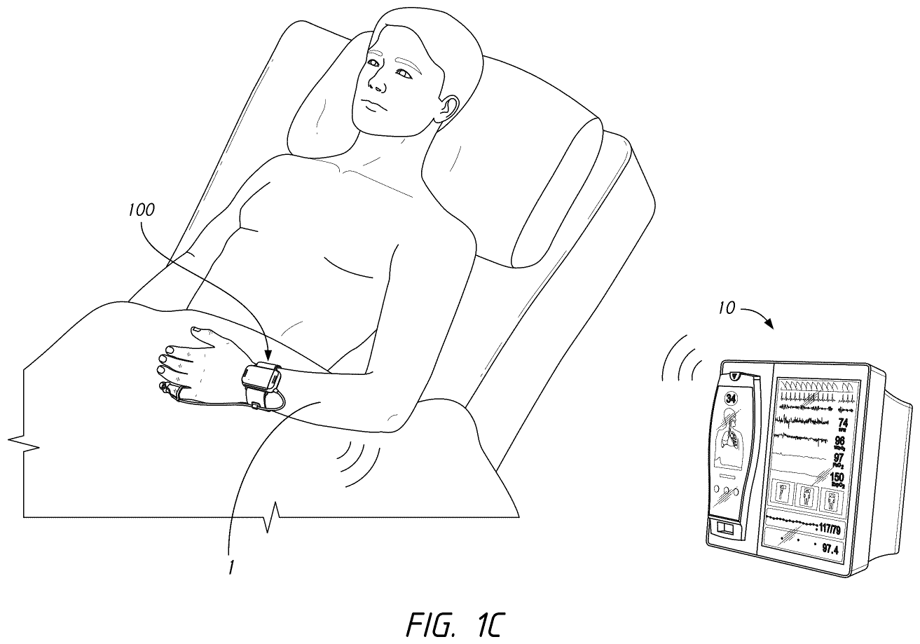

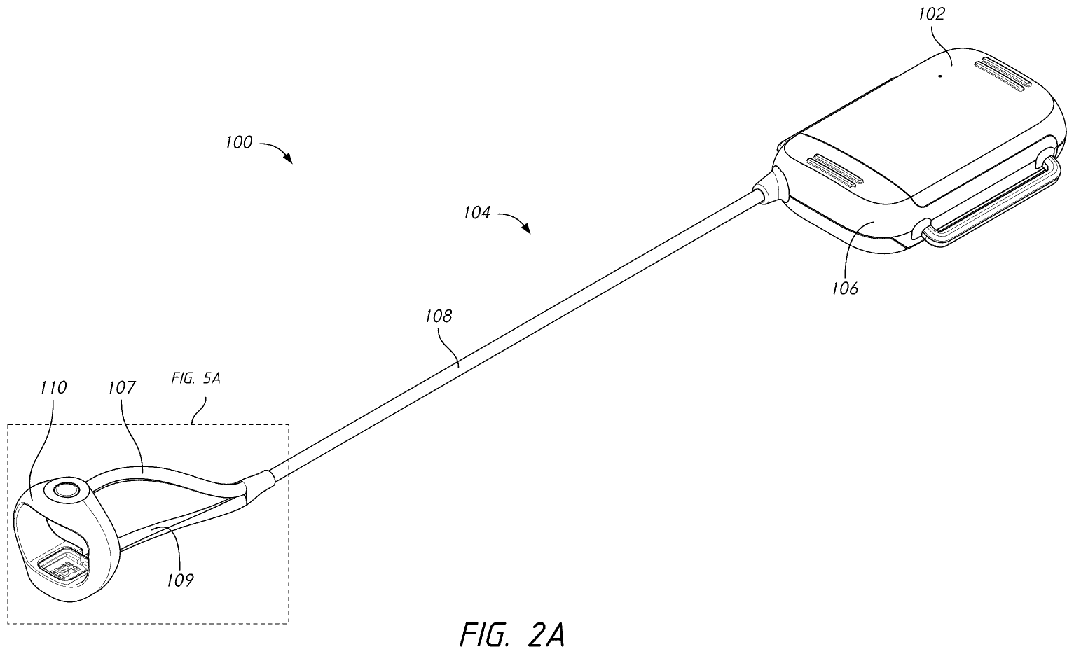

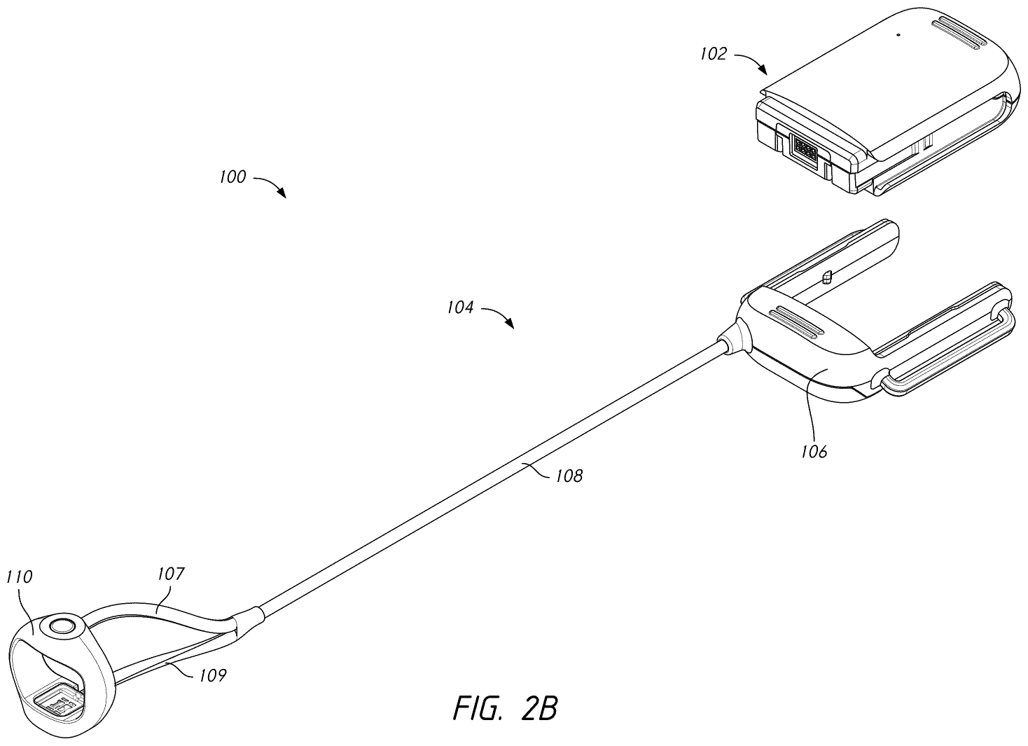

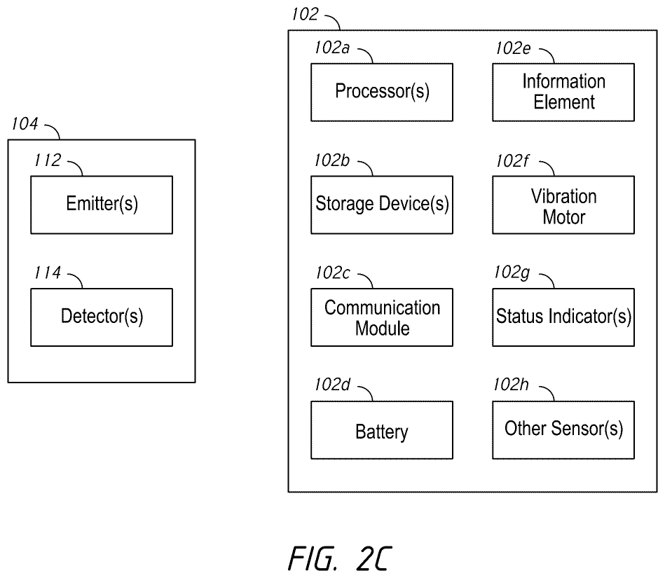

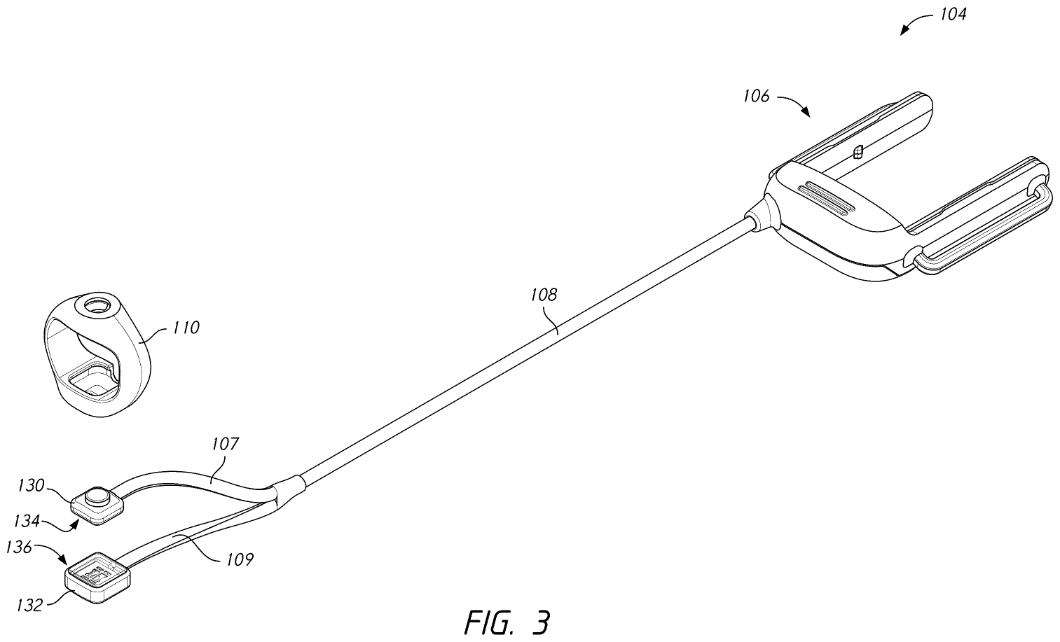

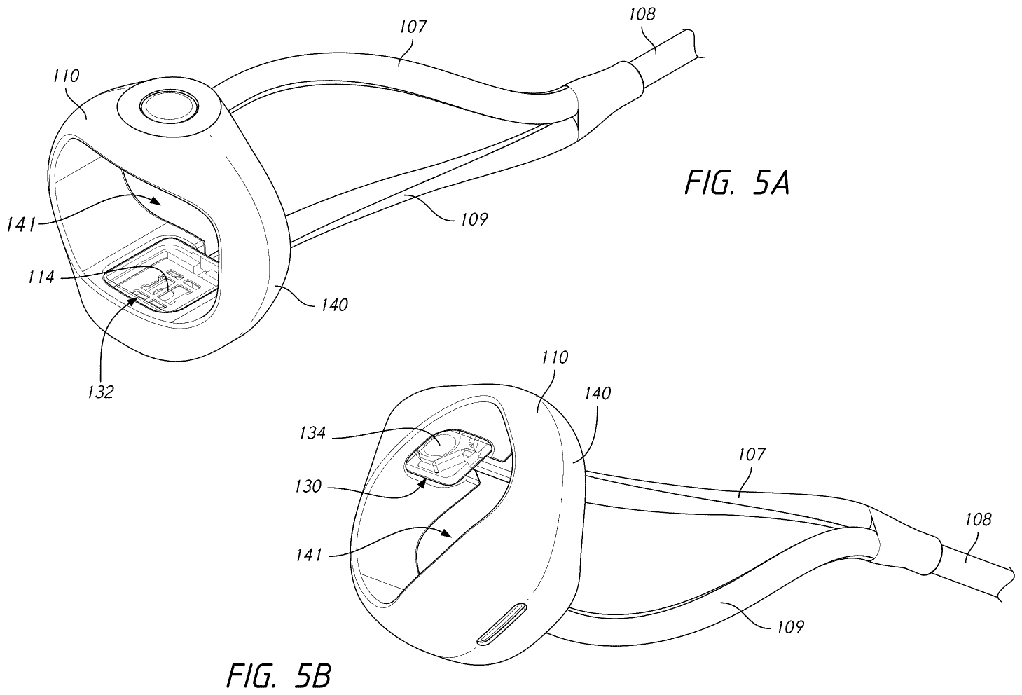

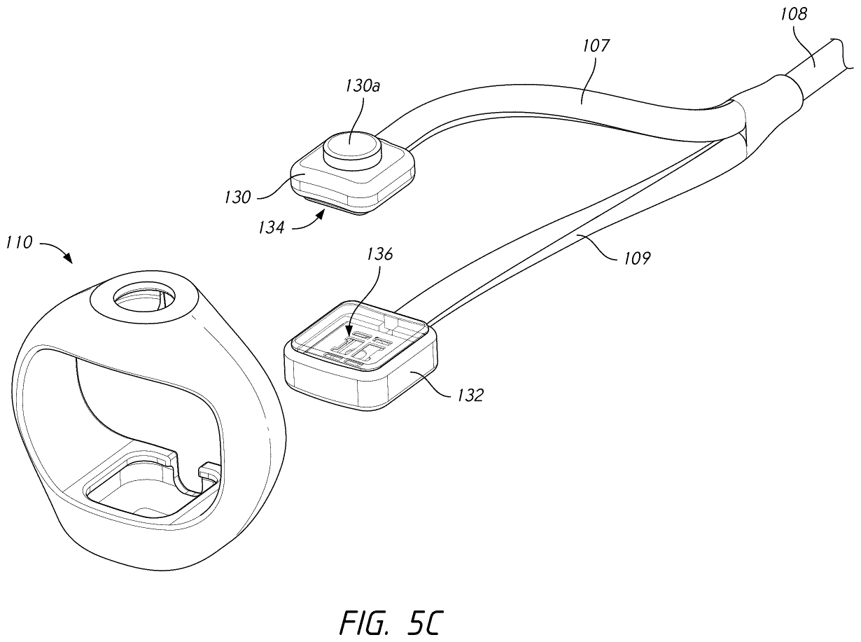

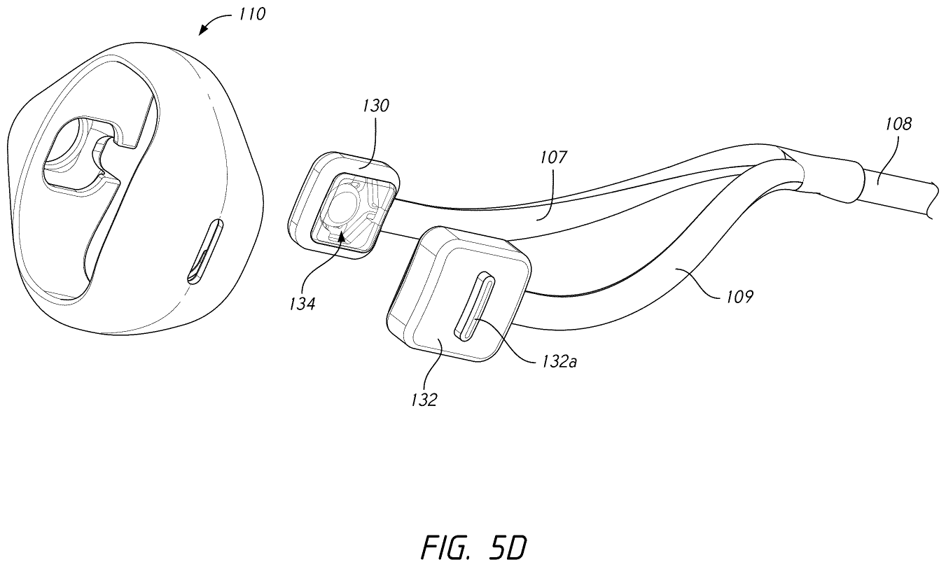

FIGS. 2A-2B illustrate perspective views of the pulse oximetry system 100 of FIG. 1A. As shown, the pulse oximetry system 100 can include a sensor dock assembly 104 and a sensor hub 102. As discussed in more detail below and as shown in FIGS. 2A-2B, the sensor hub 102 can be removably connectable to the sensor dock assembly 104. The sensor dock assembly 104 can include a dock 106, a cable 108, and a ring member 110. The sensor dock assembly 104 can additionally include one or more emitters (for example, emitter(s) 112 discussed with reference to FIG. 2C) and one or more detectors (for example, detector(s) 114 discussed with reference to FIG. 2C) to allow for pulse oximetry as further described herein. In some implementations, the pulse oximetry system 100 can include the dock 106, the cable 108, the ring member 110, one or more emitters, one or more detectors, and the sensor hub 102. As described in more detail below, the ring member 110 can secure to a finger 2 of a subject 1 and can operably position one or more emitters and one or more detectors relative to the finger 2. The cable 108 can extend between the ring member 110 and the dock 106. The cable 108 can be mechanically and electrically connected to and extend from the dock 106 and be in electrical communication with the one or more emitters 112 and the one or more detectors 114 of the sensor dock assembly 104. In some implementations and as shown in FIGS. 2A-2B, the cable 108 can have a first end connected to the dock 106 and a second end opposite such first end which can be positioned adjacent and/or can engage the ring member 110. In some implementations, such second end of the cable 108 splits into a first portion 107 and a second portion 109, with the first portion 107 connecting to the one or more emitters 112 and the second portion 109 connecting to the one or more detectors 114. In some implementations, the pulse oximetry system 100 includes an emitter package 134 (shown in FIG. 3) comprising the one or more emitters 112. Similarly, in some implementations the pulse oximetry system 100 includes a detector package 136 (shown in FIG. 3) comprising the one or more detectors 114. Further, in some implementations the pulse oximetry system 100 includes an emitter housing 130 (shown in FIG. 5B) configured to contain the emitter package 134 comprising the one or more emitters 112. The ring member 110 can be configured to receive the emitter housing 130 to operably position the one or more emitters 112. Similarly, in some implementations the pulse oximetry system 100 includes a detector housing 132 (shown in FIG. 5A) configured to contain the detector package 136 comprising the one or more detectors 114. The ring member 110 can be configured to receive the detector housing 132 to operably position the one or more detectors 114.

FIG. 2C illustrates a schematic diagram of certain features which can be incorporated in pulse oximetry system 100 as well as any other implementations of pulse oximetry sensor(s) described herein. As shown, the sensor dock assembly 104 can include one or more emitters 112 and one or more detectors 114. Also shown, the sensor hub 102 can include one or more processors 102a, one or more storage devices 102b, a communication module 102c, a battery 102d, an information element 102e, a vibration motor 102f, one or more status indicators 102g, and/or one or more other sensors 102h.

The one or more emitters 112 and the one or more detectors 114 of the pulse oximetry system 100 can be utilized to obtain physiological information indicative of one or more physiological parameters of the subject. These parameters can include various blood analytes such as oxygen, carbon monoxide, methemoglobin, total hemoglobin, glucose, proteins, glucose, lipids, a percentage thereof (for example, concentration or saturation), and the like. The one or more emitters 112 and the one or more detectors 114 of the pulse oximetry system 100 can also be used to obtain a photoplethysmograph, a measure of plethysmograph variability, pulse rate, a measure of blood perfusion, and the like. Information such as oxygen saturation (SpO₂), pulse rate, a plethysmograph waveform, respiratory effort index (REI), acoustic respiration rate (RRa), EEG, ECG, pulse arrival time (PAT), perfusion index (PI), pleth variability index (PVI), methemoglobin (MetHb), carboxyhemoglobin (CoHb), total hemoglobin (tHb), and/or glucose, can be obtained from pulse oximetry system 100 and data related to such information can be processed and/or transmitted by the pulse oximetry system 100 (for example, via communication module 102c) to a separate computing device 10 (such as a computing device at a caregiver's workstation, a patient monitor, and/or a mobile phone). The one or more emitters 112 and the one or more detectors 114 can be optically based and, for example, utilize optical radiation. Further, the one or more emitters 112 can serve as a source of optical radiation that can be directed towards tissue of the subject 1 when the pulse oximetry system 100 is in use. The pulse oximetry system 100 can include one, two, three, four, five, six, seven, or eight or more emitters 112 and/or one, two, three, four, five, six, seven, or eight or more detectors 114. The one or more emitters 112 can be one or more light-emitting diodes (LEDs) (for example, such as low-power, high-brightness LEDs), laser diodes, incandescent bulbs with appropriate frequency-selective filters, and/or any other source(s) of optical radiation and/or any combinations of the same, or the like. The one or more emitters 112 can emit optical radiation of one or more wavelengths and can emit visible and near-infrared optical radiation. The one or more emitters 112 can be similar or identical to any of the emitters discussed in U.S. Pat. No. 9,277,880, filed Jul. 1, 2010, titled “MULTI-STREAM DATA COLLECTION SYSTEM FOR NONINVASIVE MEASUREMENT OF BLOOD CONSTITUENTS,” which is hereby incorporated by reference in its entirety and for all purposes. The one or more detectors 114 can be configured to detect optical radiation generated by the one or more emitters 112. The one or more detectors 114 can detect optical radiation that attenuates through and/or is reflected by tissue of the subject 1, for example, tissue of the subject's finger 2. The one or more detectors 114 can output one or more signals responsive to the detected optical radiation. In some implementations, the one or more detectors 114 can be one or more photodiodes, phototransistors, or the like. The one or more detectors 114 can be similar or identical to any of the detectors discussed in U.S. Pat. No. 9,277,880 incorporated by reference herein.

The one or more processors 102a can be configured, among other things, to process data, execute instructions to perform one or more functions, and/or control the operation of the pulse oximetry system 100. For example, the one or more processors 102a can control operation of the one or more emitters 112, the one or more detectors 114, and/or the one or more other sensors 102h of the pulse oximetry system 100. As another example, the one or more processors 102a can process signals and/or physiological data received and/or obtained from the one or more detectors 114 and/or the one or more other sensors 102h of the pulse oximetry system 100. Further, the one or more processors 102a can execute instructions to perform functions related to storing and/or transmitting such signals and/or physiological data received and/or obtained from the one or more detectors 114 and/or the one or more other sensors 102h of the pulse oximetry system 100. The processor 102a can execute instructions to perform functions related to storing and/or transmitting any or all of such received data.

The one or more storage devices 102b can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. Such stored data can be processed and/or unprocessed physiological data obtained from the pulse oximetry system 100, for example.

The communication module 102c can facilitate communication (via wires and/or wireless connection) between the pulse oximetry system 100 (and/or components thereof) and separate devices, such as separate monitoring, computing, electrical, and/or mobile devices. For example, the communication module 102c can be configured to allow the pulse oximetry system 100 to wirelessly communicate with other devices, systems, and/or networks over any of a variety of communication protocols. The communication module 102c can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. The communication module 102c can allow data and/or instructions to be transmitted and/or received to and/or from the pulse oximetry system 100 and separate computing devices. The communication module 102c can be configured to transmit (for example, wirelessly) processed and/or unprocessed physiological parameters, data and/or other information to one or more separate computing devices, which can include, among others, a patient monitor, a mobile device (for example, an iOS or Android enabled smartphone, tablet, laptop), a desktop computer, a server or other computing or processing device for display and/or further processing, among other things. Such separate computing devices can be configured to store and/or further process the received physiological parameters, data, and/or other information, to display information indicative of or derived from the received parameters, data, and/or information, and/or to transmit information—including displays, alarms, alerts, and notifications—to various other types of computing devices and/or systems that can be associated with a hospital, a caregiver (for example, a primary care provider), and/or a user (for example, an employer, a school, friends, family) that have permission to access the subject's data. As another example, the communication module 102c of the pulse oximetry system 100 can be configured to wirelessly transmit processed and/or unprocessed obtained physiological parameters, data, information and/or other information (for example, motion and/or location data) to a mobile phone which can include one or more hardware processors configured to execute an application that generates a graphical user interface displaying information representative of the processed or unprocessed physiological parameters, data, information and/or other information obtained from the pulse oximetry system 100. The communication module 102c can be and/or include a wireless transceiver.

The battery 102d can provide power for hardware components of the pulse oximetry system 100 described herein. The battery 102d can be rechargeable. For example, the battery 102d can be a lithium, a lithium polymer, a lithium-ion, a lithium-ion polymer, a lead-acid, a nickel-cadmium, or a nickel-metal hydride battery. In some implementations, the battery 102d can be non-rechargeable. Additionally or alternatively, the pulse oximetry system 100 can be configured to obtain power from a power source that is external to the pulse oximetry system 100. For example, the pulse oximetry system 100 can include or can be configured to connect to a cable which can itself connect to an external power source to provide power to the pulse oximetry system 100.

The information element 102e can be a memory storage element that stores, in non-volatile memory, information used to help maintain a standard of quality associated with the pulse oximetry system 100. Illustratively, the information element 102e can store information regarding whether the pulse oximetry system 100 has been previously activated and whether the pulse oximetry system 100 has been previously operational for a prolonged period of time, such as, for example, four hours, one day, two days, five days, ten days, twenty days, a month, multiple months, or any period of time. The information stored in the information element 102e can be used to help detect improper re-use of the pulse oximetry system 100, for example.

The vibration motor 102f can be configured to vibrate one or more portions of the pulse oximetry system 100 (for example, the sensor hub 102 and/or the dock 106 when sensor hub 102 and dock 106 are coupled together), which in turn can vibrate one or more portions of a subject's body when the pulse oximetry system 100 is secured to the subject. For example, vibration motor 102f can be configured to vibrate the sensor hub 102 or portions thereof. The one or more processors 102a can be in communication with vibration motor 102f and can be configured to instruct vibration motor 102f to cause any of such above-described vibration. In some cases, the vibration motor 102f can be utilized to provide haptic feedback to the subject when the pulse oximetry system 100 is secured to the subject. In some implementations, the pulse oximetry system 100 can be configured to cause vibration of and/or provide haptic feedback to one or more portions of the subject's body (when the pulse oximetry system 100 is secured to the subject) via the vibration motor 102f responsive to one or more physiological parameters determined by pulse oximetry system 100 and/or by any devices (such as separate computing, electrical, and/or mobile devices, for example, a patient monitor 10) in communication with the pulse oximetry system 100. The one or more processors 102a can instruct the vibration motor 102f to cause vibration, cease vibrating, and/or instruct the vibration motor 102f to alter a characteristic of vibration (for example, increase/reduce vibration rate, increase/reduce vibration strength, change vibration pattern, etc.) responsive to the one or more determined physiological parameters. Such action by the one or more processors 102a can dynamically track with physiological parameter determination over time, for example. As an example, in some implementations, the one or more processors 102a can provide instructions to vibration motor 102f (such as those discussed above) responsive to a condition of the subject using the pulse oximetry system 100. For example, if one or more physiological parameters determined by the pulse oximetry system 100 and/or any devices in communication with the pulse oximetry system 100 are indicative of hypoxemia (low blood oxygen) when the subject is using the pulse oximetry system 100, the one or more processors 102a can instruct the vibration motor 102f to vibrate to cause the subject to wake up in an attempt to restore proper breathing and/or safe blood oxygen levels. As another example, if one or more physiological parameters determined by the pulse oximetry system 100 and/or any devices in communication with the pulse oximetry system 100 are indicative of edema (swelling caused by excess fluid trapped in body tissue) when the subject is using the pulse oximetry system 100, the one or more processors 102a can instruct the vibration motor 102a to cause vibration of a portion of the subject's body, such as their wrist, forearm, hand, ankle, lower leg, and/or any other portion of the subject's body. In some implementations, the one or more processors 102a and/or any devices in communication with the pulse oximetry system 100 can instruct the vibration motor 102f to cause a vibration if a determined subject physiological parameter of interest meets and/or exceeds a set threshold, meets and/or falls below a set threshold, and/or meets, exceeds, and/or falls below a set range. In some cases, a vibration of the vibration motor 102f can correspond to an alert, an alarm, a notification, and/or any other situation wherein the subject and/or a care provider can need to intervene in the subject's care. In some implementations, the one or more processors 102a can instruct the vibration motor 102f to vibrate responsive to a status of battery 102d (for example, when a charge of the battery 102d drops below a certain threshold). In some implementations, pulse oximetry system 100 can include more than one vibration motor 102f, for example, two, or three or more vibration motors 102f. Vibration motor(s) 102f can be positioned within various portions of the pulse oximetry system 100, for example, within sensor hub 102.

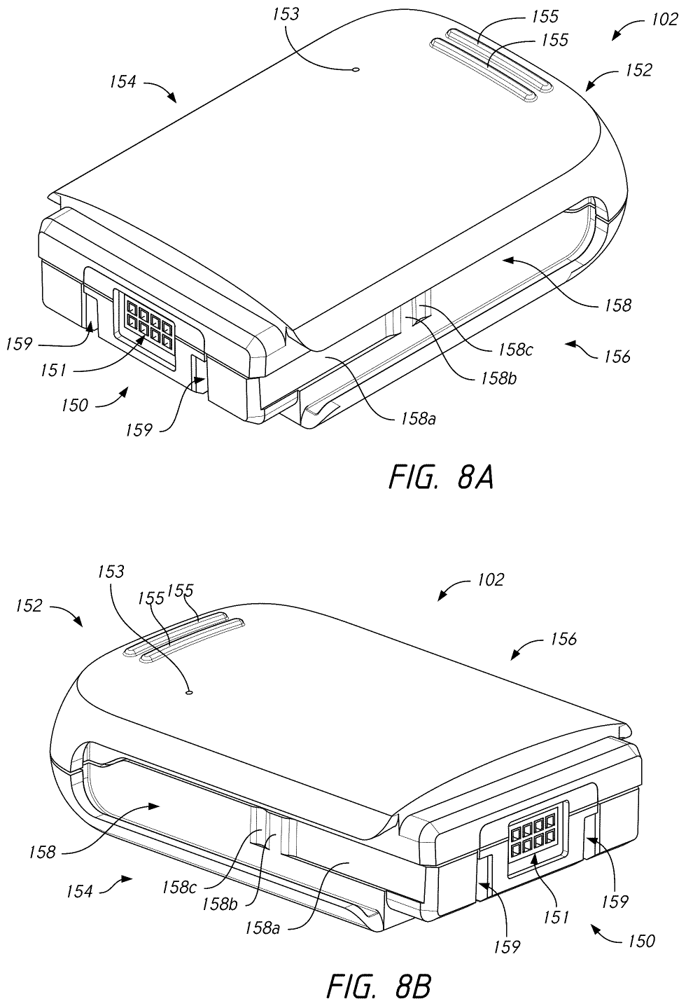

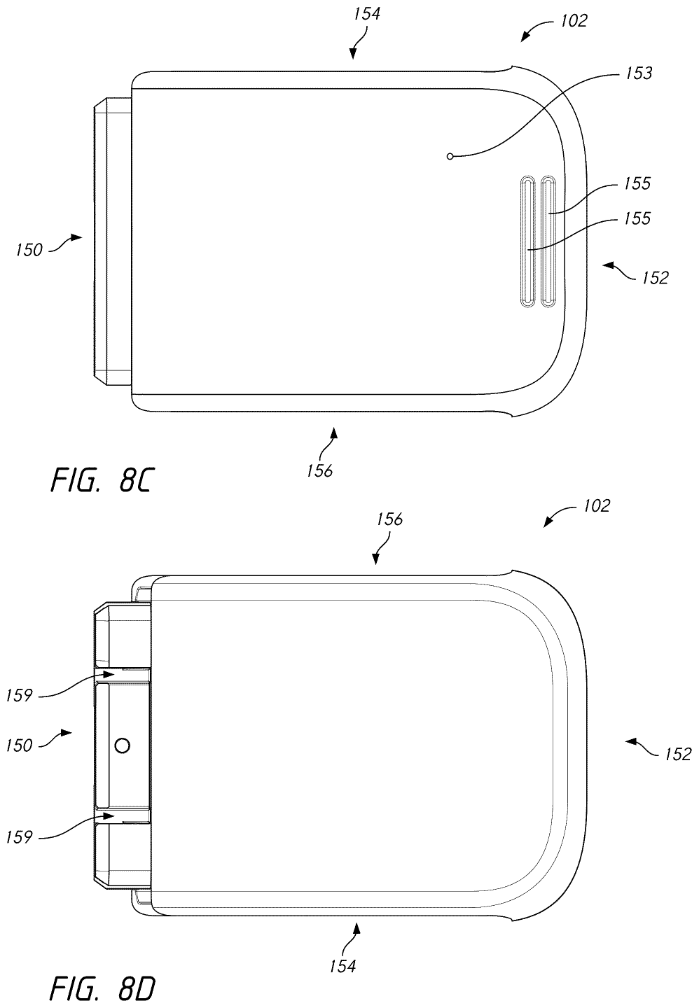

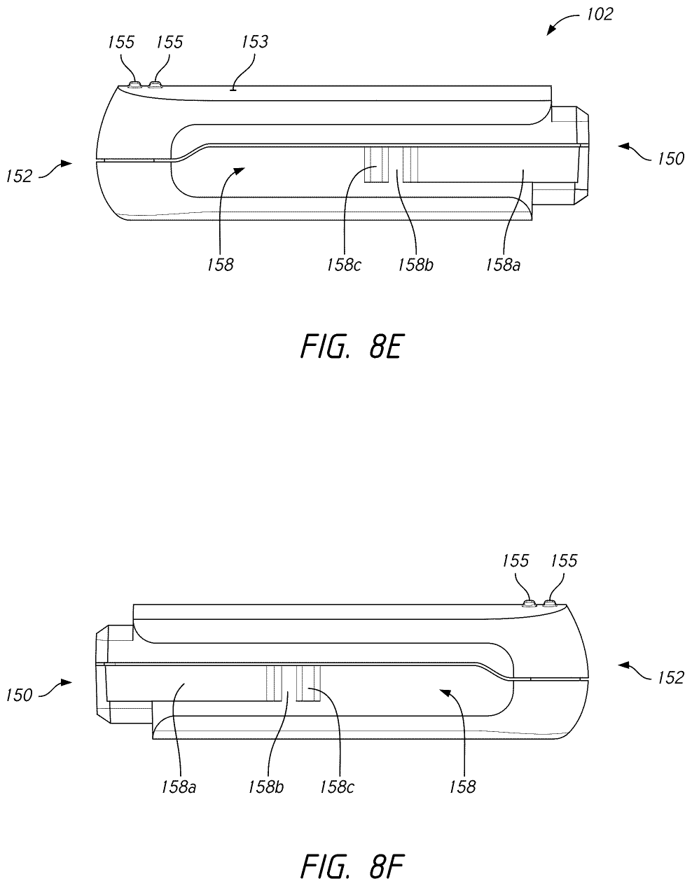

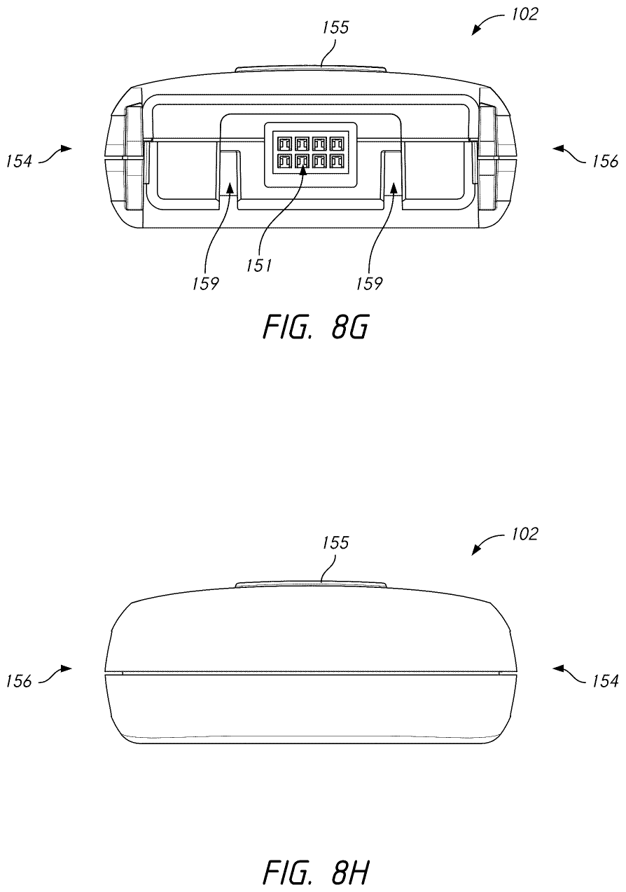

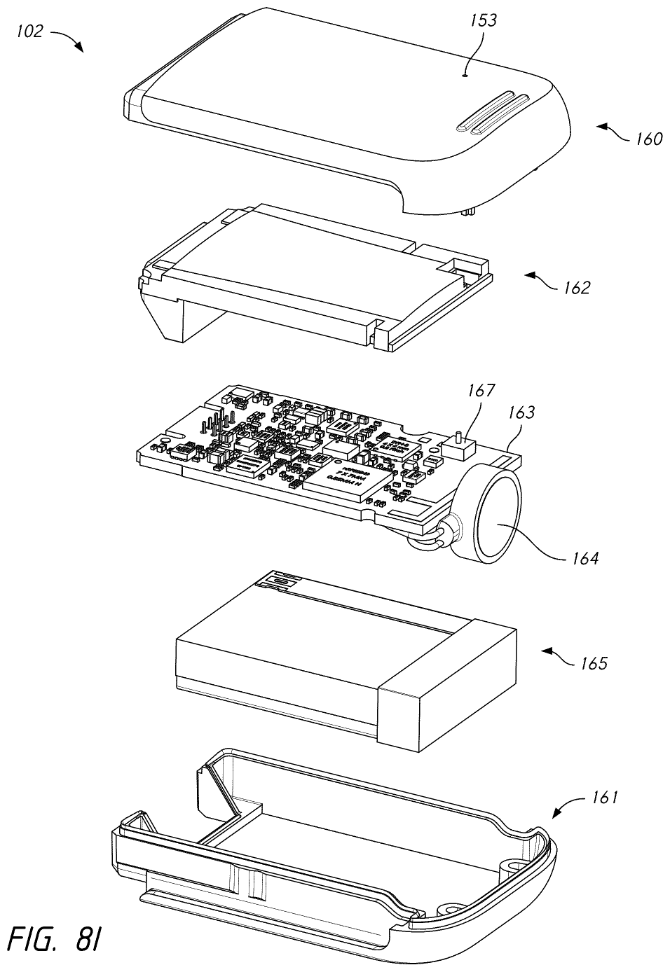

The one or more status indicators 102g can be configured to provide and/or indicate a status of the pulse oximetry system 100 and/or a status of one or more physiological parameters of the subject 1 determined by the pulse oximetry system 100 and/or any devices in communication with the pulse oximetry system 100. In some implementations, the one or more status indicators 102g can be configured to indicate a status of the pulse oximetry system 100, such as whether the pulse oximetry system 100 is in an operational (“on”) mode, whether the pulse oximetry system 100 is pairing or has paired with a separate device, whether an error has been detected, and/or a power level of the pulse oximetry system 100 (for example, a charge of battery 102d of sensor hub 102). For example, the one or more status indicators 102g can be configured to light up and/or cast optical radiation of one or more wavelengths from one or more portions of the pulse oximetry system 100. As another example, the one or more status indicators 102g can be configured to light up and/or emit optical radiation from one or more portions of the sensor hub 102 of the pulse oximetry system 100. The one or more processors 102a can be in communication with the one or more status indicators 102g and can be configured to instruct the one or more status indicators 102g to cause any of such above-described status indications and/or lighting. In some cases, the one or more status indicators 102g can be configured to provide optical radiation (e.g., light) feedback to the subject when the pulse oximetry system 100 is secured to the subject and/or when sensor hub 102 and dock 106 are connected together. In some implementations, pulse oximetry system 100 can be configured to cause optical radiation feedback to the subject 1 (when the pulse oximetry system 100 is secured to the subject) responsive to one or more physiological parameters determined by pulse oximetry system 100 and/or by any devices (such as separate computing and/or mobile devices, for example, a patient monitor) in communication with the pulse oximetry system 100. The one or more processors 102a can instruct the one or more status indicators 102g to emit or stop emitting optical radiation and/or instruct the one or more status indicators 102g to alter a characteristic of optical radiation (for example, increase/reduce optical radiation brightness, change optical radiation wavelength and/or color, change a rate of blinking of optical radiation, etc.) responsive to the one or more determined physiological parameters. Such action by the one or more processors 102a can dynamically track with physiological parameter determination over time, for example. As an example, in some implementations, the one or more processors 102a can provide instructions to the one or more status indicators 102g (such as those discussed above) responsive to a condition of the subject using the pulse oximetry system 100. For example, if one or more physiological parameters determined by the pulse oximetry system 100 and/or any devices in communication with the pulse oximetry system 100 are indicative of hypoxemia (low blood oxygen) when the subject is using the pulse oximetry system 100, the one or more processors 102a can instruct the one or more status indicators 102g to produce optical radiation to notify the subject and/or their care providers to restore proper breathing and/or safe blood oxygen levels. As another example, if one or more physiological parameters determined by the pulse oximetry system 100 and/or any devices in communication with the pulse oximetry system 100 are indicative of edema (swelling caused by excess fluid trapped in body tissue) when the subject is using the pulse oximetry system 100, the one or more processors 102a can instruct the one or more status indicators 102g to cause optical radiation to be emitted from the pulse oximetry system 100 as described above. In some implementations, the one or more processors 102a and/or any devices in communication with the pulse oximetry system 100 can instruct the one or more status indicators 102g to cause optical radiation to be emitted if a determined subject physiological parameter of interest meets and/or exceeds a set threshold, meets and/or falls below a set threshold, and/or meets, exceeds, and/or falls below a set range. In some cases, optical radiation emitted from the one or more status indicators 102g can correspond to an alert, an alarm, a notification, and/or any other situation wherein the subject and/or a care provider can need to intervene in the subject's care. The one or more status indicators 102g can be positioned within various portions of the pulse oximetry system 100, for example, within sensor hub 102, such that optical radiation emitted from the one or more status indicators emit out of and/or through a hole and/or opening in the sensor hub 102, such as by status indicator 167 shown in and described with respect to FIGS. 8I and 8J through hole 153 of the sensor hub 102 shown and described with respect to FIGS. 8A, 8B, and 8C.

In some implementations, the pulse oximetry system 100 can include one or more other sensor(s) 102h. The other sensor(s) 102h can include one or more of a temperature sensor, an electrocardiogram (ECG) sensor, a blood pressure monitor, an acoustic sensor, and/or any sensor configured to obtain physiological information indicative of one or more physiological parameters of the subject. Additionally, or alternatively, such other sensor(s) 102h can comprise a motion sensor, for example, including one or more accelerometers and/or gyroscopes, that can be utilized to determine motion and/or position of the subject and/or a portion of the subject's body (for example, wrist 3 and/or finger 2). In some implementations where the pulse oximetry system 100 (for example, sensor hub 102) includes a motion sensor, the processor(s) 102a can determine whether the subject's wrist, hand, and/or finger(s) are moving and, responsive to such determination, not receive, not process, and/or not determine one or more physiological parameters (since such determinations can include inaccuracies because of such movement). The other sensor(s) 102h can be disposed on, within, and/or be operably positioned by any one or more of the aspects of the pulse oximetry system 100. For example, the other sensor(s) can be disposed on, within, and/or be operably positioned by any one or more of the dock 106, the cable 108, and/or the ring member 110 of the sensor dock assembly 104, and/or by the sensor hub 102. As another example, the other sensor(s) can be disposed on, within, and/or be operably positioned by any one or more of the cable retainers of the pulse oximetry system 100 (such as cable retainers 103, 105 described later herein) if included. The other sensor(s) 102h can be operably connected to the one or more processors 102a, which can control operation of the other sensor(s) 102h and/or process data received from the other sensor(s) 102h.

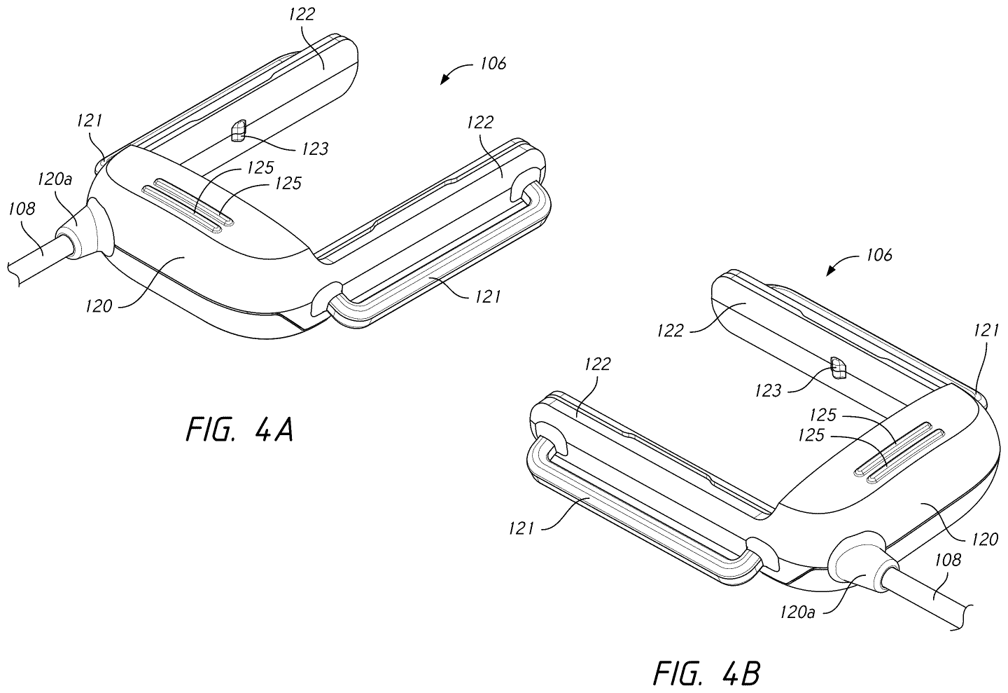

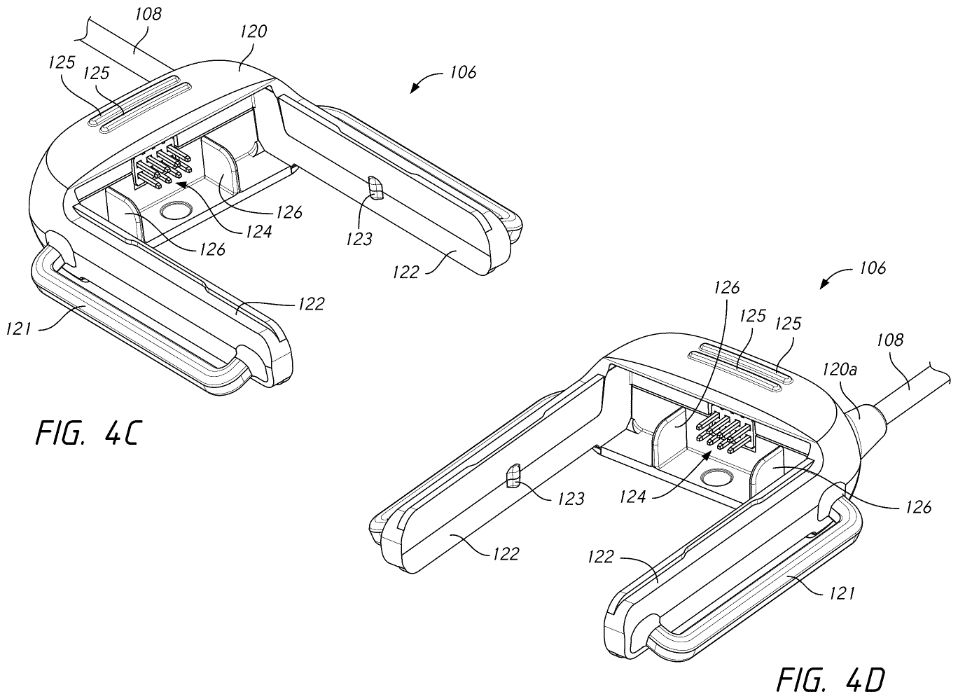

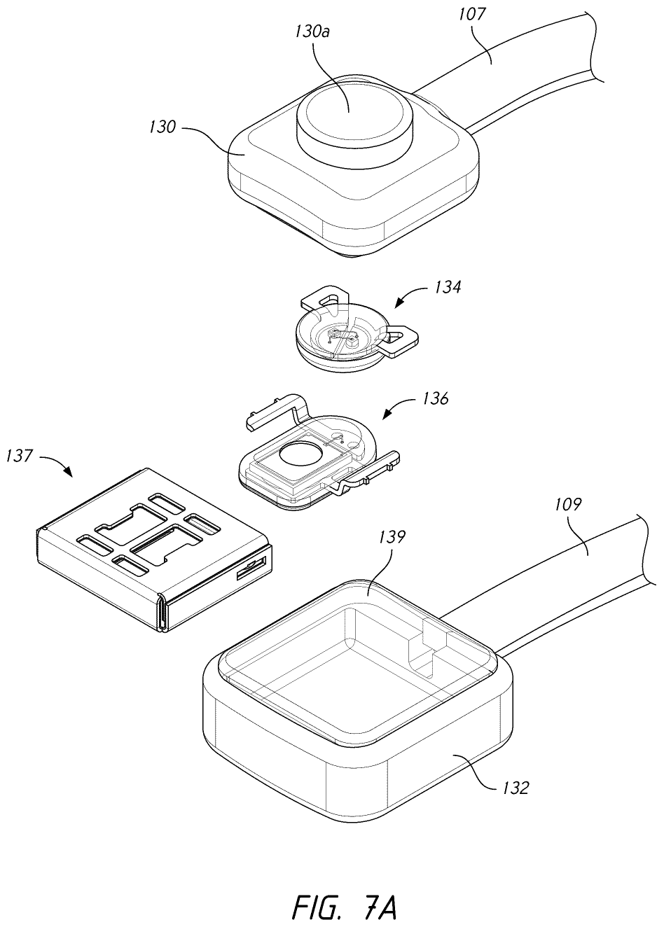

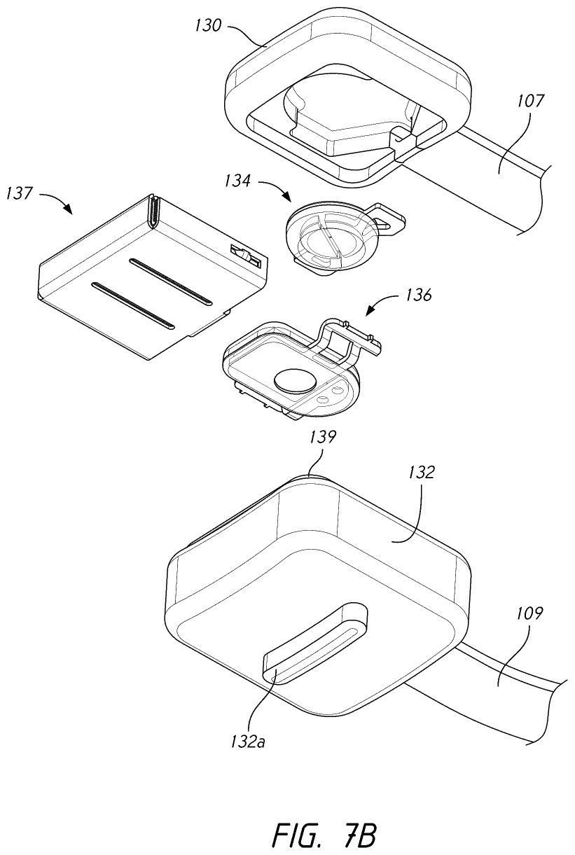

FIG. 3 illustrates a perspective view of the sensor dock assembly 104 of the pulse oximetry system 100 shown in FIGS. 1A-1C and FIGS. 2A-2B with the ring member 110 removed from the cable 108 and the sensor hub 102 removed from the dock 106. Also shown, and as described above relative to FIGS. 2A-2B, the cable 108, at an end opposite of an end where it connects to the dock 106, can split into a first portion 107 and a second portion 109, with the first portion 107 connecting to the one or more emitters 112 of emitter package 134 and the second portion 109 connecting to the one or more detectors 114 of detector package 136. As mentioned above, an emitter housing 130 can house the emitter package 134 (see FIGS. 7A-7B) that can include the one or more emitters 112, and as such the emitter housing 130 can be connected with the first portion 107 of cable 108 as shown. Similarly and as mentioned above, a detector housing 132 can house the detector package 136 (see FIGS. 7A-7B) that can include the one or more detectors 114, and as such the detector housing 132 can be connected with the second portion 109 of cable 108 as shown. Exemplary implementations of the sensor dock assembly 104 and its components will be described below through FIGS. 4A-4D, 5A-5D, 6A-6J, and 7A-7B.

FIGS. 4A-4D illustrate perspective views of the dock 106 of the sensor dock assembly 104. As shown, the dock 106 can include a base 120 (which can also be referred to herein as a “housing”) and arm(s) 122 extending outward from the base 120. The dock 106 can be mechanically and electrically connected to the cable 108 as discussed above through a bend relief 120a disposed on a portion of and/or extending outward from the base 120. The dock 106 can be electrically coupled to the one or more emitters 112 in emitter package 134 and the one or more detectors 114 in detector package 136 of the pulse oximetry system 100 via the connection to cable 108 through bend relief 120a. The base 120 can include an electrical connector 124 configured to releasably electrically connect the dock 106 (and therefore the cable 108 along with the one or more emitters 112 and the one or more detectors 114) to the sensor hub 102 (not shown). The dock 106 can also include features for mechanically engaging/connecting with the sensor hub 102. For example and as shown, the dock 106 can include arm(s) 122 extending from the base 120. The arm(s) 122 can be configured to releasably mechanically engage/connect with the sensor hub 102 (not shown). In some implementations, when the sensor hub 102 (not shown) is mechanically engaged/connected with the arm(s) 122 of the dock 106, an electrical connector of the sensor hub 102 (for example, electrical connector 151 shown in FIGS. 8A-8B) can releasably mechanically and electrically engage/connect with the electrical connector 124 of the dock 106. The dock 106 can include one or more features configured to secure the dock 106 to the subject 1. For example, as shown in FIGS. 4A-4D, the dock 106 can include one or more strap loop(s) 121 configured to be coupled to a strap (such as strap 101 shown in FIGS. 1A-1B). Such strap loop(s) 121 can be connected to arm(s) 122 as shown and strap 101 can be inserted through and/or around strap loop(s) 121 and around a portion of the subject's body (for example, wrist 3) such that the strap 121 secures the dock 106 to the subject's wrist 3. The dock 106 can include two strap loop(s) 121. The two strap loop(s) 121 can each be disposed along an outer surface of the arm(s) 122 (e.g., disposed on a side opposite of where the sensor hub 102 can engage/connect with the arm(s) 122), such that each arm 122 has a strap loop 121 connected to it and such that each strap loop 121 runs in a direction generally parallel to the arm 122 that it is connected to. As shown, the strap loop(s) 121 can be configured to create a generally elongated opening configured to couple to one or more straps 101 for securing the pulse oximetry system 100 to the subject 1. In some cases, the dock 106 can include one strap loop 121 and a strap configured to secure at one end to such strap loop 121 and at its other end directly to the dock 106. In some implementations, the dock 106 does not include strap loop(s) 121. In some variants, the dock 106 can be secured to the wrist 3 in an alternative manner. For example, in some variants, the dock 106 can be configured to secure to the subject 1 without strap 101, for example, via an adhesive material disposed on a portion of the dock 106 that can secure to the subject's skin, via tape, via a wrap-around strap, or via any other type or form of securement. In some implementations, a strap 101 can be integrated with the dock 106 such that strap loop(s) 121 are not necessary. In some implementations, the dock 106 can include one or more features for aiding in gripping and/or holding the dock 106, such as for gripping and/or holding the dock 106 when connecting and/or disconnecting sensor hub 102 to the dock 106. For example, the dock 106 can include one or more ribs 125 disposed on a portion of the base 120 of the dock 106, the ribs 125 configured to aid in gripping and/or holding the dock 106. The ribs 125 can include generally linear protrusions that protrude out from the surface of the dock 106 and extend along a portion of the dock opposite of where the dock would contact the subject 1 when secured to the subject 1 (e.g., the ribs can be disposed along an “upper” portion of the dock 106). Alternatively, or in addition, in some implementations the dock 106 can include other features configured to aid in gripping and/or holding the dock 106, such as bumps, a roughened surface texture, etc. Further details of exemplary features of the dock 106 are described below.

As shown in FIGS. 4A-4D, in some implementations, the dock 106 has a length and/or a width that are greater than a height of the dock 106. In some implementations, the dock 106 includes two arm(s) 122. The arm(s) 122 of the dock 106 can extend from the base 120 in the same direction such that the dock 106 has a generally U-shaped structure. The arm(s) 122 can be generally parallel to each other, such that a gap is formed between the arm(s) 122. Such a gap can be, for example, sized to accommodate the sensor hub 102 and/or at least a portion of the sensor hub 102. In some implementations, the arm(s) 122 are the same length. Furthermore, the arm(s) 122 can mirror each other in size, shape, and other features. In some implementations, the dock 106 can include one or more retaining features configured to engage the sensor hub 102. For example, each of the arm(s) 122 of the dock 106 can include a protrusion 123 configured to engage with the sensor hub 102 to allow the dock 106 to connect to the sensor hub 102. The protrusion(s) 123 can be disposed along an inner surface of each of the arm(s) 122, such that they face towards the sensor hub 102 when the sensor hub 102 is connected to the dock 106. The protrusion(s) 123 can smoothly transition from the inner surface(s) of the arm(s) 122 such that the sensor hub 102 can slidably engage with the protrusion(s) 123. For example, the protrusion(s) 123 can include ramp-like structures that define a smooth transition between the inner surface of the arm(s) 122 and the maximum “height” of the protrusion(s) 123. As another example, the protrusion(s) 123 can be rounded, have a rounded tip, and/or have a parabolic cross-section that allow for a smooth transition with the inner surface of the arm(s) 122. The protrusion(s) 123 can interact with corresponding recess(es) in the sensor hub 102, which can serve to releasably lock the sensor hub 102 in place with the dock 106. In some implementations, interaction between the protrusion(s) 123 of the dock 106 and recess(es) of the sensor hub 102 can provide tactile feedback to the subject that indicates complete engagement/connection of the sensor hub 102 with the dock 106. Details of such recess(es) of the sensor hub 102 are described later relative to FIGS. 8A-8B and 8E-8F. Any number of retaining features can be provided on the dock 106 to aid in releasably connecting the sensor hub 102 to the dock 106. In some cases, other types of retaining features can be utilized. For example, edges of the arm(s) 122, which can be defined as the transition between the inner surface of the arm(s) 122 and outer surfaces of the arm(s) 122, can be configured to aid in the connection between the dock 106 and the sensor hub 102. In some implementations, the arm(s) 122 are sized and shaped to releasably connect to the sensor hub 102. In some cases, the sensor hub 102 slidably connects to the dock 106.

As discussed above and as shown in FIGS. 4A-4D, the dock 106 can include a bend relief 120a configured to mechanically and electrically couple/connect the cable 108 of the pulse oximetry system 100 to the dock 106. In some implementations, the bend relief 120a can be disposed on a portion of the base 120 and extend away from the base 120 in a direction generally opposite of the arm(s) 122. The bend relief 120a can extend from any portion of the dock 106 and in any direction from the dock 106. The bend relief 120a can be disposed on a portion of the dock 106 such that it does not interfere with the sensor hub 102 releasably coupling with the dock 106. In some implementations, the bend relief 120a can be disposed on a portion of the dock 106 such that it extends out from the dock in a direction substantially parallel to the arm(s) 122. In some cases, the bend relief 120a can be disposed adjacent a corner of the dock 106 and extend from the dock 106 in any direction. In some implementations, for example, as shown in FIGS. 4A-4B, the bend relief 120a can be disposed adjacent a corner of the dock 106 and extend from the corner in a direction generally opposite the direction the arm(s) 122 extend from the dock. The bend relief 120a can advantageously extend from the dock 106 and position the cable 108 substantially parallel to a portion of the subject's body when the dock 106 is secured to the subject 1 (e.g., such that the cable 108 is substantially parallel to the subject's forearm, allowing for a low-profile).

As discussed above and as shown in FIGS. 4C-4D, the dock 106 can include an electrical connector 124. The electrical connector 124 can be configured to releasably electrically and mechanically connect to a corresponding electrical connector of the sensor hub 102, such that when connected to each other, the sensor hub 102 is placed in electrical communication with the one or more emitters 112 and the one or more detectors 114 of the pulse oximetry system 100. The electrical connector 124 can include any number of pins. For example, with reference to FIGS. 4C-4D, the electrical connector 124 can include 8 pins. In some implementations, the electrical connector 124 includes an alternative number of pins. In some implementations, the electrical connector 124 can include a number of openings that correspond to a number of pins of a corresponding electrical connector of the sensor hub 102. As shown in FIGS. 4C-4D, the electrical connector 124 can be disposed at an inner portion of the base 120 of the dock 106, such that it faces the sensor hub 102 when the sensor hub 102 is connected to the dock 106. In some implementations, the dock 106 can include one or more features to aid in aligning the electrical connector 124 to the corresponding electrical connector of the sensor hub 102. For example, the dock 106 can include walls 126 positioned adjacent the electrical connector 124, and the walls 126 can be configured to aid in releasably connecting the electrical connector 124 with the corresponding electrical connector of the sensor hub 102.

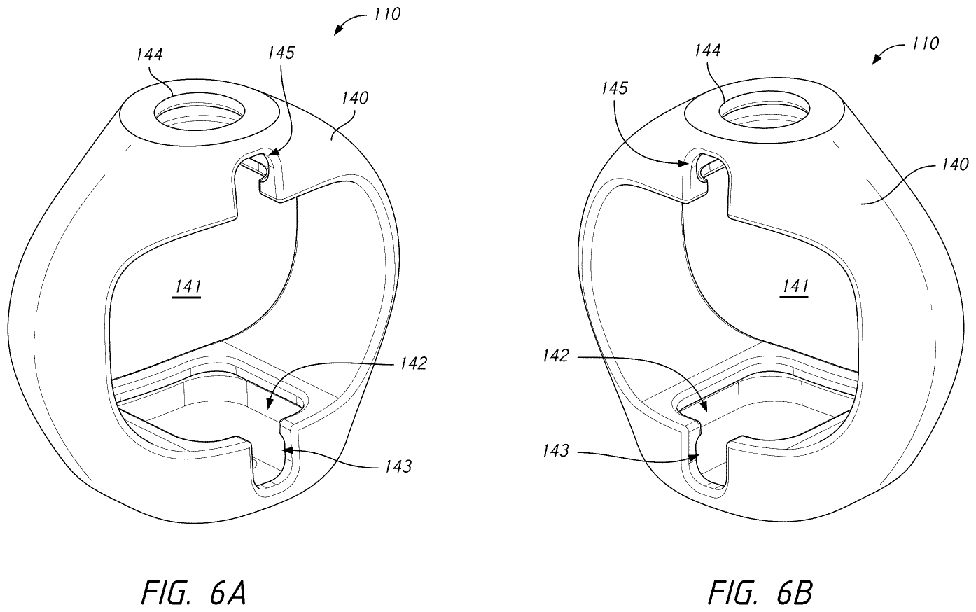

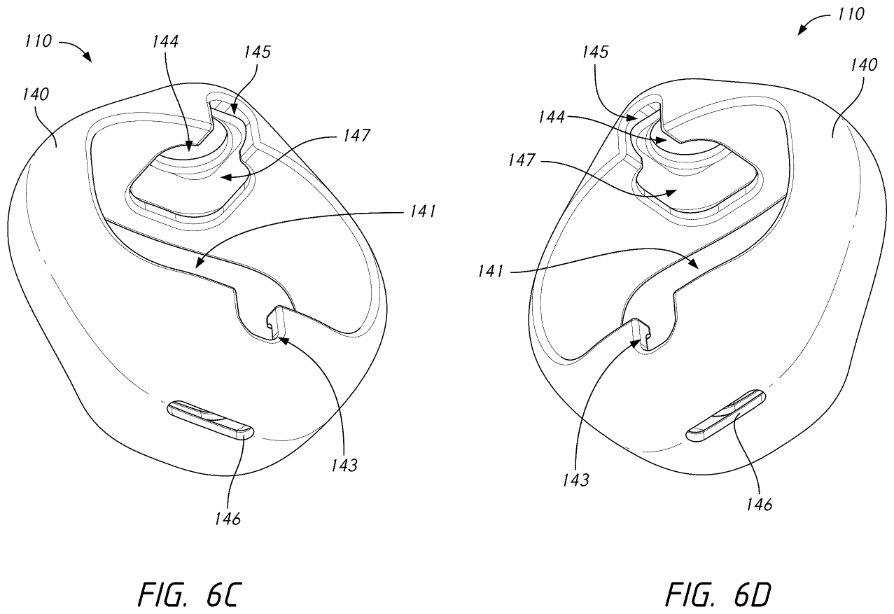

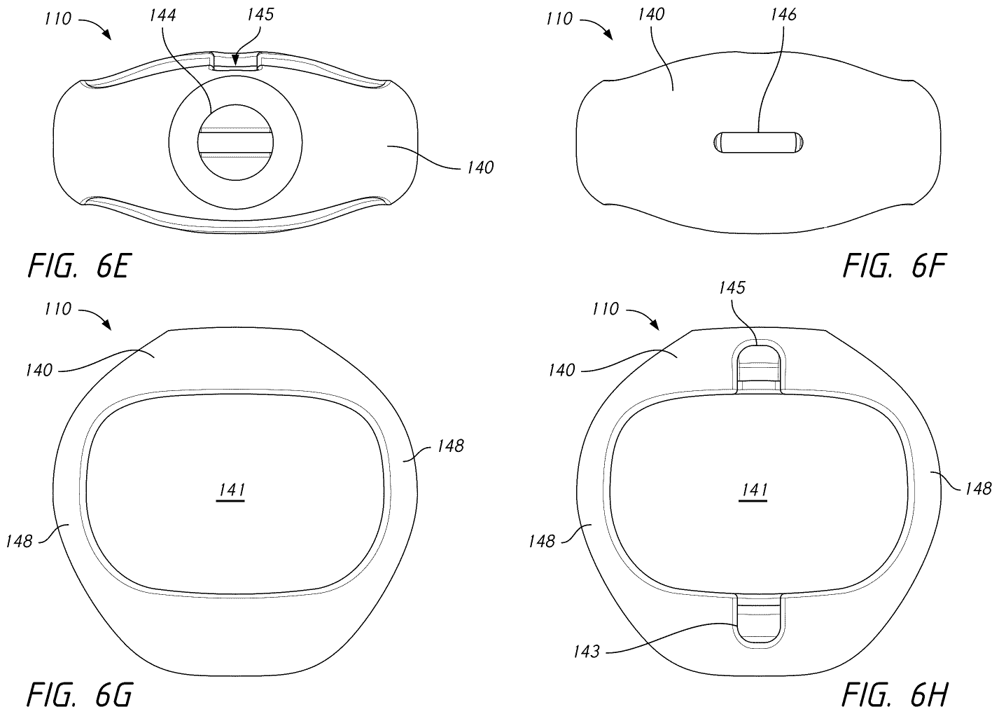

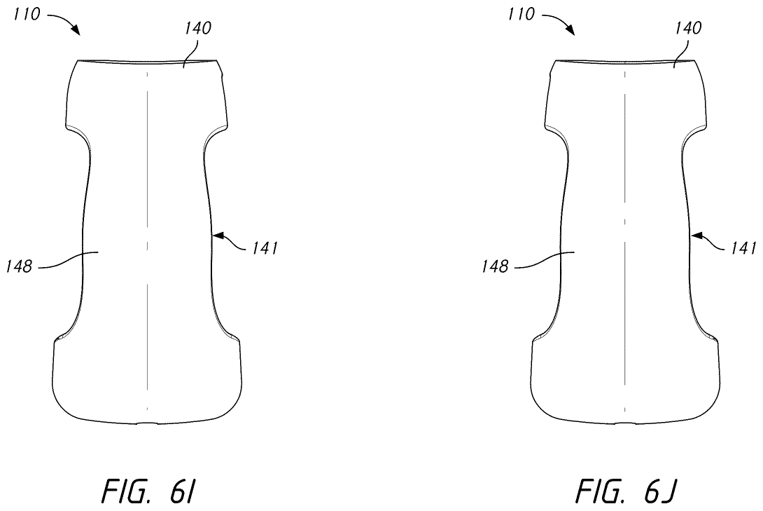

FIGS. 5A-5B illustrate perspective views of the cable 108 splitting into the first portion 107 and the second portion 109, with the first portion 107 connected to the emitter package 134 within emitter housing 130 operably positioned by the ring member 110 and the second portion 109 connected to the detector package 136 within detector housing 132 operably positioned by the ring member 110. The ring member 110 (which is described further below with respect to FIGS. 6A-6J) can comprise a body 140 with an opening 141 configured to receive a portion of the subject's finger 2 therethrough. For example, the opening 141 can be configured to receive a portion of a subject's pinky finger therethrough, such as shown in FIGS. 1A-1C. The ring member 110 can operably position the one or more emitters 112 of the emitter package 134 by positioning the emitter housing 130 such that the one or more emitters 112 are directed to and/or face the opening 141 of the ring member 110. In such a position, the emitter housing 130 can be positioned against and/or adjacent to tissue of the subject's finger, such as a nail bed of the subject's finger. Similarly, the ring member 110 can operably position the one or more detectors 114 of the detector package 136 by positioning the detector housing 132 such that the one or more detector 114 are directed to and/or face the opening 141 of the ring member 110. In such a position, the detector housing 132 can be positioned against and/or adjacent to tissue of the subject's finger. As shown in FIGS. 5A-5B, the detector housing 132 can be positioned by a portion of the ring member 110 opposite a portion that positions the emitter housing 130. Furthermore, in some implementations the detector housing 132 can be positioned on a side of the opening 141 of the ring member 110 that is opposite a side of the opening 141 where the emitter housing 130 is positioned. Thus, detector package 136 can be operably positioned by the ring member 110 such that optical radiation emitted from the emitter package 134 can pass through and/or be attenuated by the tissue and/or the nail of the subject's finger 2 before being detected by the detector package 136. In some implementations, the ring member 110 operably positions the emitter housing 130 and the detector housing 132 such that the one or more emitters 112 and the one or more detectors 114 are generally aimed at one another. In some implementations, the ring member 110 operably positions the emitter housing 130 and the detector housing 132 such that the one or more emitters 112 and the one or more detectors 114 are substantially aligned with one another. As discussed herein, in some implementations, one or more ring members 110 can be provided, each having an opening 141 configured to receive a portion of the subject's finger 2 therethrough. The size of the opening 141 of such one or more ring members 110 can each be different so as to accommodate different sized fingers and/or different portions of a finger. In some implementations, a kit can include one or more of any of the components of the pulse oximetry system 100. For example, a kit can include one or more ring members 110 sized to accommodate fingers and/or portions of fingers of different sizes and any of the features described with respect to pulse oximetry system 100 (and/or any of the other pulse oximetry systems described herein).

Also shown in FIGS. 5A-5B, the first portion 107 and second portion 109 of the cable 108 can extend away from each other after splitting off from the cable 108. Such extension away from each other can advantageously allow a finger 2 of the subject 1 to pass through a gap made between the first portion 107 and the second portion 109. Further shown and in some implementations, the first portion 107 and/or the second portion 109 of the cable 108 can include one or more bends that can allow the first portion 107 and/or the second portion 109 to operably connect to the emitter package 134 within the emitter housing 130 and to the detector package 136 within the detector housing 132, respectively, when the ring member 110 is secured around at least a portion of the subject's finger 2. Further exemplary implementations of how the cable 108, the first portion 107, and the second portion 109 can be routed and/or positioned along the subject 1 are discussed further below. In some implementations (not shown), the cable 108 does not split into first and second portions but can instead connect as a singular cable to the ring member 110 (for example, to a side and/or end of the ring member 110, such a side and/or end of the ring member 110 that faces towards the dock 106 when the pulse oximetry system 100 is worn by/secured to the subject 1).

FIGS. 5C-5D illustrate perspective views of the emitter housing 130, the first portion 107 of cable 108, the detector housing 132, and the second portion 109 of cable 108 removed from the ring member 110. The ring member 110 can advantageously be configured to removably receive the emitter housing 130 and the detector housing 132. For example, and as will be discussed further below with respect to FIGS. 6A-6J, the ring member 110 can comprise a resilient and flexible material that can allow for the removable securement of the emitter housing 130 and the detector housing 132. Such configuration can allow for a ring member 110 of various sizes and/or shapes to be utilized with the pulse oximetry system 100, for example, so as to accommodate various sizes and/or shapes of a subject's finger 2. In this way, the pulse oximetry system 100 can be customized to a subject 1 by selecting an appropriately configured ring member 110 while allowing for all other aspects of the pulse oximetry system 100 to remain the same and/or be universal across subjects.

With continued reference to FIGS. 5C-5D and as discussed above, the emitter housing 130 can be configured to contain the emitter package 134 comprising the one or more emitters 112. Similarly, the detector housing 132 can be configured to contain the detector package 136 comprising the one or more detectors 114. In some implementations, the emitter housing 130 can be of a different shape and/or size than the detector housing 132. Such different shape and/or size between the emitter housing 130 and the detector housing 132 can help and/or aid in distinguishing how to place the housings into the ring member 110. For example, a different shape and/or size between the emitter housing 130 and the detector housing 132 can index the housings to the ring member 110. In some implementations, the emitter housing 130 and/or the detector housing 132 can include additional features for positioning and/or securing the housings within the ring member 110. For example and as shown in FIG. 5C, the emitter housing 130 can include a protrusion 130a extending from the emitter housing 130. The protrusion 130a can be of any shape and/or size, and can be configured to fit into a corresponding feature and/or opening of the ring member 110 when the emitter housing 130 is positioned by the ring member 110. For example and as shown, the protrusion 130a can be a cylindrically shaped protrusion extending from a portion of the emitter housing 130 opposite of where the emitter package 134 emits optical radiation. In some implementations and as shown in FIG. 5D, the detector housing 132 can include a protrusion 132a extending from the detector housing 132. The protrusion 132a can be of any shape and/or size, and can be configured to fit into a corresponding feature and/or opening of the ring member 110 when the detector housing 132 is positioned by the ring member 110. For example and as shown, the protrusion 132a can be an elongate protrusion extending from a portion of the detector housing 132 opposite of where the detector package 136 detects optical radiation emitted from the emitter package 134.

FIGS. 6A-6D illustrate perspective views of the ring member 110, FIG. 6E illustrates a top view of the ring member 110, FIG. 6F illustrates a bottom view of the ring member 110, FIGS. 6G-6H illustrate front and back views, respectively, of the ring member 110, and FIGS. 6I-6J illustrate side views of the ring member 110. As discussed previously and as shown, the ring member 110 can include a body 140 with an opening 141 configured to receive a portion of the subject's finger therethrough. Thus, in some implementations, the ring member 110 can be shaped in the form of a ring configured to secure around at least a portion of the subject's finger 2.

As described herein, the ring member 110 can operably position the emitter package 134 and the detector package 136 of the pulse oximetry system 100. As such, the ring member 100 can include one or more features, openings, cavities, and/or the like configured to operably position the emitter package 134 and the detector package 136. For example, in some implementations and as shown, the ring member 110 can include a cavity 147 sized and shaped to receive the emitter housing 130 described previously. The ring member 110 can also include a cavity 142 sized and shaped to receive the detector housing 132 described previously. The cavity 147 can have a different size and/or shape than the cavity 142, which can aid in positioning the emitter housing 130 and the detector housing 132 into the respective cavity 147 and cavity 142. The cavity 147 can open into and/or join with the opening 141 of the ring member 110. Similarly, the cavity 142 can open into and/or join with the opening 141 of the ring member 110. The cavity 147 can be disposed opposite the cavity 142 so as to operably position the emitter package 134 and the detector package 136 when the cavity 147 receives the emitter housing 130 and the cavity 142 receives the detector housing 132.

In some implementations, the ring member 110 can include one or more additional features, openings, cavities, and/or the like configured to position the emitter housing 130, the detector housing 132, and/or portions thereof. For example, the ring member 110 can include a cavity opening 144 sized and shaped to receive the protrusion 130a of the emitter housing 130. The cavity opening 144 can open into and/or join with the cavity 147. Similarly, the ring member 110 can include a cavity opening 146 sized and shaped to receive the protrusion 132a of the detector housing 132. The cavity opening 146 can open into and/or join with the cavity 142. As shown and in some implementations, the cavity opening 144 can be a generally cylindrical opening corresponding to the cylindrically shaped protrusion 130a. Further as shown and in some implementations, the cavity opening 146 can be a generally elongate opening corresponding to the elongate protrusion 132a. The cavity openings 144 and 146 can extend completely through or partially through the body 140 of the ring member 110 (as shown, both cavity openings extend completely through). When the emitter housing 130 is disposed within the cavity 147 of the ring member 110, the protrusion 130a of the emitter housing 130 can be positioned within the cavity opening 144. Similarly, when the detector housing 132 is disposed within the cavity 142 of the ring member 110, the protrusion 132a of the detector housing 132 can be positioned within the cavity opening 146.

With continued reference to FIGS. 6A-6J, the ring member 110 can include one or more notches configured to receive one or more portions of the cable 108 of the pulse oximetry system 100. For example, the ring member 110 can include a notch 145 configured to receive the first portion 107 of cable 108. As another example, the ring member 110 can include a notch 143 configured to receive the second portion 109 of cable 108. Notches 145 and 143 can be disposed on an end of the ring member 110 that faces towards the dock 106 when the ring member 110 is secured to the subject's finger 2 and the dock 106 is secured to the subject's wrist 3 (e. g, the back of the ring member 110). The notch 145 can extend through an end of the ring member 110 and open into the cavity 147 so as to allow the first portion 107 of the cable 108 to operably connect with the emitter package 134 within emitter housing 130 when the emitter housing 130 is positioned by the ring member 110. Similarly, the notch 143 can extend through an end of the ring member 110 and open into the cavity 142 so as to allow the second portion 109 of the cable 108 to operably connect with the detector package 136 within detector housing 132 when the detector housing 132 is positioned by the ring member 110. In some implementations, notches 143 and 145 can open into and/or join with opening 141 of the ring member 110, which can facilitate removal and/or insertion of the emitter housing 130 and the detector housing 132 from and/or into ring member 110.

Advantageously, the ring member 110 can be made of a resilient and flexible material. For example, the ring member 110 can be made of silicone, such as a medical grade and/or biocompatible silicone, a thermoplastic elastomer, such as a medical grade and/or biocompatible thermoplastic elastomer, and/or any biocompatible material and/or polymer that is resilient and flexible. In some implementations, ring member 110 comprises silicone rubber. A resilient and flexible ring member 110 can advantageously position the emitter housing 130 and the detector housing 132 close to, against, and or adjacent to a portion of the subject's body, such as the nail bed and/or the tissue of the subject's finger, for optimal function of the pulse oximetry system 100. For example, by having flexibility to conform to the subject's finger 2, the ring member 110 can optimally position the emitter package 134 against, adjacent, and/or near to the subject's finger 2 such that optical radiation emitted from the emitter package 134 is directed to/through the subject's finger 2. By way of another example, by the ring member 110 having the flexibility to conform to the subject's finger 2, ambient and/or stray optical radiation and/or optical radiation not produced/emitted by the emitter package 134 can be reduced, eliminated, and/or prevented from being received by the detector package 136. Furthermore, a resilient and flexible ring member 110 can advantageously improve comfort for the subject 1 when the ring member 110 is secured to and/or worn by the subject 1. In some implementations, the ring member 110 comprises a rigid material. In some implementations, ring member 110 comprises a first material and a second material that is more rigid than the first material.

The body 140 of the ring member 110 can have a variable thickness. For example, as shown in FIGS. 6G-6H, sides 148 of the ring member 110 (positioned adjacent opening 141) can have a reduced thickness compared to portions of the ring member 110 configured to position the emitter housing 130 and the detector housing 132. Such a configuration can increase subject comfort when the ring member 110 is secured around the subject's finger 2 given the ring member 110 can interact with adjacent fingers of the subject (e.g., with a reduced thickness at sides 148 of the ring member 110, the subject may not “feel” as much of a foreign body sensation between their fingers when wearing the ring member 110). As shown in FIGS. 6I-6J, the body 140 of the ring member 110 can have a variable side profile. For example, the sides 148 of the ring member 110 can have a width that is less than the portions of the ring member 110 configured to position the emitter housing 130 and the detector housing 132. As shown, the side profile of the body 140 of the ring member 110 can be generally “I” shaped. Such a configuration can increase subject comfort when the ring member 110 is secured around the subject's finger 2. Furthermore, such a configuration can increase the pliability and/or conformability of the ring member 110 and aid in optimally operably positioning the emitter package 134 and the detector package 136 when the ring member 110 is secured around the subject's finger 2. In some cases, the ring member 110 can be provided in various and/or different sizes to accommodate various finger sizes.

In some implementations, the ring member 110 has a generally ring-like shape, however the ring member 110 can be of other shapes and/or configurations. For example, the ring member 110 can be configured as an incomplete ring, such that opening 141 is not fully enclosed by the body 140. In some implementations, the ring member 110 can be configured in any shape for operably securing the emitter package 134 and the detector package 136 at opposite sides of the subject's finger 2. In some implementations, the ring member 110 can be configured to inhibit, prevent, and/or reduce an amount of ambient light, stray light, and/or any optical radiation not emitted from the emitter package 134 from reaching the detector package 136. Additionally, or alternatively, the ring member 110 can be configured to inhibit, prevent, and/or reduce an amount of optical radiation emitted by the emitter package 134 that has not been attenuated by, reflected by, and/or passed through tissue of the subject from being received by the detector package 136. In some cases, the ring member 110 can be opaque and/or generally light blocking and/or have a light blocking coating. In some implementations, ring member 110 can be semi-transparent or transparent. In some implementations, the ring member 110 can include portions that are opaque and/or light blocking and portions that are semi-transparent and/or transparent.

FIGS. 7A-7B illustrate perspective exploded views of aspects of emitter(s) and detector(s) of the pulse oximetry system 100. As discussed relative to FIGS. 5C-5D, the emitter housing 130 can be configured to contain the emitter package 134 comprising the one or more emitters 112, and the detector housing 132 can be configured to contain the detector package 136 comprising the one or more detectors 114. The emitter package 134 is shown removed from the emitter housing 130 and the detector package 136 is shown removed from the detector housing 132. Also shown is a detector shield 137, which can be included with the detector package 136 in the detector housing 132. The detector shield 137 can at least partially enclose and/or surround the detector package 136 comprising the one or more detectors 114. The detector shield 137 can be configured to inhibit, prevent, and/or reduce ambient light, stray light, and/or light emitted from the emitter package 134 that does not pass through tissue from being received by the detector package 136, which can advantageously improve the integrity of physiological parameter determination. Additionally, or alternatively, detector shield 137 can shield the detector package 136 against and/or with respect to electromagnetic noise. For example, in some implementations, the detector shield 137 can act as a Faraday cage or a shield to block electromagnetic fields. Also shown are other features and/or aspects that can be included in and/or comprise part of the emitter housing 130 and/or the detector housing 132. For example and as shown in FIG. 7A, the detector housing 132 can include optical transmission material 139. In some implementations, the optical transmission material 139 can be configured to direct optical radiation toward the detector package 136 after passing through and/or being reflected by tissue of the subject's finger 2. In some cases, the optical transmission material 139 can include a lens. In some cases, the optical transmission material 139 can include a diffuser configured to diffuse, spread out, disseminate, and/or scatter optical radiation attenuated by and/or reflected from tissue prior to being received by the detector package 136. The optical transmission material 139 can form a part of the detector housing 132. Further, the optical transmission material 139 can be configured to be positioned between the detector package 136 and tissue of the subject 1 when the pulse oximetry system 100 is secured to the subject 1. By way of another example and as shown in FIG. 7B, the emitter housing 130 can include optical transmission material 131. In some implementations, the optical transmission material 131 can be configured to focus or diffuse optical radiation emitted from the emitter package 134. In some cases, the optical transmission material 131 can include a lens. In some cases, the optical transmission material 131 can include a diffuser configured to diffuse, spread out, disseminate, and/or scatter optical radiation emitted from the emitter package 134 prior to such optical radiation entering the subject's tissue. In some cases, this can permit optical radiation emitted from the emitter package 134 to pass through a greater amount of tissue and can facilitate more accurate determination of physiological parameters (such as any of those discussed herein). The optical transmission material 131 can form a part of the emitter housing 130. Further, the optical transmission material 131 can be configured to be positioned between the emitter package 134 and tissue and/or at least a portion of a nail (e.g., the nail bed) of the subject 1 when the pulse oximetry system 100 is secured to the subject 1.

FIGS. 8A-8B illustrate perspective views, FIG. 8C illustrates a top view, FIG. 8D illustrates a bottom view, FIGS. 8E-8F illustrate side views, and FIGS. 8G-8H illustrate front and back views, respectively, of the sensor hub 102. FIGS. 8I-8J illustrate perspective exploded views of the sensor hub 102.

As shown in FIGS. 8A-8J, the sensor hub 102 can include a first end 150, a second end 152 opposite the first end 150, a first side 154, and a second side 156 opposite the first side 154. Sensor hub 102 can comprise a length along sides 154, 156 and/or a width along ends 150, 152 greater than a height of the sensor hub 102. As discussed above with respect to FIG. 2C, the sensor hub 102 can include one or more processors 102a, one or more storage devices 102b, a communication module 102c, a battery 102d, an information element 102e, a vibration motor 102f, one or more status indicators 102g, and/or one or more other sensors 102h. Further as discussed above, the sensor hub 102 can be configured to releasably mechanically and electrically connect with the dock 106 of the sensor dock assembly 104 of the pulse oximetry system 100. For this, the sensor hub 102 can be sized and/or shaped and/or include one or more features (for example, recesses) for releasably electrically and mechanically connecting to the dock 106. In some cases, the sensor hub 102 includes one or more features for engaging with one or more retaining features of the dock 106. For example, in some implementations the sensor hub 102 includes recessed portion(s) 158 disposed along at least a portion of the sides 154, 156 configured to slidably and releasably mechanically connect with the arm(s) 122 of the dock 106. The recessed portion(s) 158 can extend from end 150 along sides 154, 156 towards end 152, and in some cases can terminate adjacent to and/or near the end 152. In addition to recessed portion(s) 158, the sensor hub 102 can include one or more features for releasably mechanically connecting to the dock 106. For example, the sensor hub 102 can include recess(es) 158a, non-recessed portion(s) 158b, and recess(es) 158c, which can as shown be disposed along recessed portion(s) 158. In some implementations, the recess(es) 158a and the recess(es) 158c can be configured to slidably receive the protrusion(s) 123 of the dock 106 when the recessed portion(s) 158 of the sensor hub 102 slidably engage with the arm(s) 122 of the dock 106. For example, the sensor hub 102 can be mechanically connected to the dock 106 by aligning recessed portion(s) 158 with arm(s) 122 of the dock 106 while the sensor hub 102 is positioned away from the dock 106 but generally in the same plane as the dock 106 and with end 150 of the sensor hub 102 facing the dock 106, sliding the sensor hub 102 towards the dock 106 such that the arm(s) 122 engage with the recessed portion(s) 158, continuing to slide the sensor hub 102 towards the dock 106 such that the recess(es) 158a receive the protrusion(s) 123, continuing to slide the sensor hub 102 towards the dock 106 such that the non-recessed portion(s) 158b engage and/or interact with the protrusion(s) 123, and continuing to slide the sensor hub 102 towards the dock 106 until the recess(es) 158c receive the protrusion(s) 123. In the example above, the interaction between the non-recessed portion(s) 158b of the sensor hub 102 and the protrusion(s) 123 of the dock can provide tactile feedback to the subject, such as the feel of a “snap” when the non-recessed portion(s) 158b are slid past the protrusion(s) 123. In the case of connecting the sensor hub 102 to the dock 106, such a “snap” feel can indicate to the subject that the sensor hub 102 is fully connected to the dock 106 once the protrusion(s) 123 have slid past the non-recessed portion(s) 158b and the recess(es) 158c have received the protrusion(s) 123.

With continued reference to FIGS. 8A-8J and as discussed above, the sensor hub 102 can include an electrical connector 151 configured to electrically and mechanically connect with the corresponding electrical connector 124 of the dock 106. The sensor hub 102 and its components, such as processor(s) 102a and battery 102d, can be operably connected to the one or more emitters 112 and the one or more detectors 114 of the pulse oximetry system 100 when the electrical connector 151 of the sensor hub 102 is connected to the electrical connector 124 of the dock. In some implementations, the electrical connector 151 of the sensor hub 102 can be electrically and mechanically connected to the electrical connector 124 of the dock 106 when the sensor hub 102 is mechanically connected to the dock 106, such as by the connection between the arm(s) 122 of the dock and the recessed portion(s) 158 of the sensor hub 102 as described above. The electrical connector 151 of the sensor hub 102 can include one or more openings configured to receive one or more pins of the corresponding electrical connector 124 of the dock 106. For example and as shown in FIGS. 8A-8B, the electrical connector 151 of the sensor hub 102 can include 8 openings, the openings configured to electrically and mechanically connect with corresponding pins of the electrical connector 124 of the dock 106. In some implementations, the electrical connector 151 can include any number of openings. In some implementations, the electrical connector 151 can include one or more of pins configured to electrically and mechanically connect with one or more corresponding openings in the electrical connector 124 of the dock 106. As shown in FIGS. 8A-8B, the electrical connector 151 can be disposed at end 150 of the sensor hub 102, for example, such that it faces towards the electrical connector 124 of the dock 106 when the sensor hub 102 is connected to the dock 106. In some implementations, the sensor hub 102 can include one or more features to aid in aligning the electrical connector 151 to the corresponding electrical connector 124 of the dock 106. For example, the sensor hub 102 can include slot(s) 159 adjacent the electrical connector 151, the slot(s) 159 configured to releasably receive the walls 126 of the dock 106 and aid in releasably connecting the electrical connector 151 with the corresponding electrical connector 124.

As shown in FIGS. 8A-8C, 8E-8H, the sensor hub 102 can include one or more features for aiding in gripping, holding, moving, and/or sliding the sensor hub 102, such as for sliding the sensor hub 102 when connecting and/or disconnecting the sensor hub 102 to the dock 106. For example, the sensor hub 102 can include one or more ribs 155 disposed on a portion of the sensor hub 102, the ribs 155 configured to aid in gripping, holding, moving, and/or sliding the sensor hub 102. As shown, the ribs 155 can include generally linear protrusions that protrude out from a surface of the sensor hub 102 and extend at least partially from side 154 towards side 156 along a portion of the sensor hub 102 near end 152 (e.g., the ribs can be disposed along an “upper” portion of the sensor hub 102). Alternatively, or in addition, in some implementations the sensor hub 102 can include other features configured to aid in gripping, holding, moving, and/or sliding the sensor hub 102, such as bumps, a roughened surface texture, etc.

With continued reference to FIGS. 8A-8J and as discussed above, the sensor hub 102 can include one or more status indicators 102g. The one or more status indicators 102g can be configured, for example, to emit optical radiation out of and/or through a hole and/or opening in the sensor hub 102, such as through the hole/opening 153 in a top shell 160 of the sensor hub 102. As shown in FIGS. 8I-8J, the one or more status indicators 102g can include status indicator 167, which can be an emitter (e.g., an LED) configured to emit optical radiation. The status indicator 167 can be operably coupled to a circuit board (also referred to herein as a “PCB”) 163 and the processor(s) 102a of the sensor hub 102. The hole/opening 153 can allow the optical radiation emitted by the status indicator 167 to be visible from a location external to the sensor hub 102, such as by the subject 1 when wearing/using the pulse oximetry system 100. In some implementations, hole/opening 153 can be at least partially aligned with status indicator 167 to allow optical radiation emitted from the status indicator 167 to more easily pass through the top shell 160. Additionally, or alternatively, the top shell 160 and/or a bottom shell 161 of the sensor hub 102 can comprise a transparent or semi-transparent material that allows optical radiation emitted from the status indicator 167 to be seen from a location external to the sensor hub 102.

In some implementations, the sensor hub 102 can include an RFID reader and the dock 106 of the sensor dock assembly 104 can include an RFID tag. The RFID tag of the dock 106 can be configured to communicate with the RFID reader of the sensor hub 102. In some implementations, the sensor hub 102 can include an RFID tag and the dock 106 of the sensor dock assembly 104 can include an RFID reader. The RFID tag of the sensor hub 102 can be configured to communicate with the RFID reader of the dock 106.

Referring to the perspective exploded views of FIGS. 8I-8J, shown are components of the sensor hub 102 in accordance with some implementations. As shown, the sensor hub 102 can include the bottom shell 161, a battery 165, the PCB 163, and the top shell 160. In some implementations, the sensor hub 102 can also include a vibration motor 164, the status indicator 167 described above, and/or a PCB overmolding 162. The vibration motor 164 can be an example implementation of vibration motor 102f discussed herein, and can be configured to provide haptic feedback, vibration, alerts, notifications, alarms, etc. to the subject 1 when the pulse oximetry system 100 is secured to the subject. The battery 165 can correspond to battery 102d discussed herein, and can be configured to provide power to the pulse oximetry system 100. The PCB 163 can include and/or be operably coupled with the processor(s) 102a, the storage device(s) 102b, the communication module 102c, the information element 102e, the status indicator(s) 102g and status indicator 167, and/or the vibration motor 102f and vibration motor 164 discussed previously. The PCB overmolding 162 can be configured to seal the PCB 163, at least a portion of the PCB 163, and/or at least some of the PCB's components, such as against water, other liquids, air, dust, contaminants, etc. The PCB overmolding 162 can also be configured to provide shock and/or drop protection for the PCB 163 and/or its components. The battery 165 can be configured to operably connect to the PCB 163, components of the PCB 163, and/or the electrical connector 151 of the sensor hub 102. The bottom shell 161 and the top shell 160 can be configured to contain components of the sensor hub 102 and can connect/join to each other (for example, by ultrasonic welding) to create a housing/shell of the sensor hub 102. As such, the top shell 160 and/or the bottom shell 161 can comprise the one or more features of the sensor hub 102 configured to releasably connect with the dock 106, such as recessed portion(s) 158, recess(es) 158a, non-recessed portion(s) 158b, and recess(es) 158c.

Returning back to FIGS. 1A-1B, the pulse oximetry system 100 can be configured to advantageously provide increased user comfort, increased user ergonomics, facilitate better sensor-skin contact and engagement in order to provide more accurate physiological parameter determination, and/or provide better stability in securement than existing sensors. One aspect in particular that can provide at least some of the advantages listed above includes management/placement/guidance of the cable 108 that connects the dock 106 to the emitter package 134 comprising the one or more emitters 112 and the detector package 136 comprising the one or more detectors 114 operably positioned by the ring member 110. In some implementations and as shown, the pulse oximetry system 100 can include one or more cable retainers configured to secure to one or more portions of the cable 108 to retain the cable 108 in position when the pulse oximetry system 100 is used by the subject 1. For example, the pulse oximetry system 100 can include a cable retainer 103 and/or a cable retainer 105, each of which will be described further below.

The cable retainer 103 can include one or more prongs configured to secure a portion of the cable 108 (for example, as shown in FIGS. 1A-1B). The cable retainer 103 can be configured to releasably connect/secure the portion of the cable 108. In some cases, the cable retainer 103 can be configured to slidably secure the portion of the cable 108, allowing for the cable 108 to slide back and/or forth relative to the cable retainer 103. The cable retainer 103 can be attached to, adhered to, integrated with, and/or a part of the strap 101. In some implementations (not shown in FIGS. 1A-1B), the cable retainer 103 can be attached to, adhered to, integrated with, and/or a part of the dock 106.

The cable retainer 105 can comprise a ring configured to secure around a portion of the subject's finger 2 and can include one or more prongs configured to secure a portion of the cable 108 (e.g., a portion other than the portion that can be secured by cable retainer 103). The cable retainer 105 can be configured to releasably connect/secure the portion of the cable 108. In some cases, the cable retainer 105 can be configured to slidably secure the portion of the cable 108, allowing for the cable 108 to slide back and/or forth relative to the cable retainer 105. In some implementations and as shown in FIGS. 1A-1B, the cable retainer 105 can be secured around a subject's finger 2 between the ring member 110 and the subject's palm 4 and include one or more prongs configured to secure a portion of the cable 108. For example, the cable retainer 105 can be secured around a subject's finger 2 at a base of the finger where it meets the subject's palm 4 (e.g., between the palm and a first joint of the finger). In some implementations, the cable retainer 105 can be configured to slide along the finger 2 of the subject. In some implementations (not shown), the cable retainer 105 can comprise a feature other than a ring for securing to/around the subject's finger 2, such as a clip, a partial ring, a C-shaped member, etc. The part(s) of the cable retainer 105 that secure to/around the subject's finger 2 can be flexible, soft, and/or have aspects that can enhance comfort to the subject when worn. In some implementations, at least a portion of the cable retainer 105 can be made of silicone, such as a medical grade and/or biocompatible silicone, a thermoplastic elastomer, such as a medical grade and/or biocompatible thermoplastic elastomer, and/or any biocompatible material and/or polymer that is resilient and/or flexible. In some implementations, the cable retainer 105 comprises silicone rubber. In some implementations, the cable retainer 105 comprises a rigid material. In some implementations, cable retainer 105 comprises a first material and a second material that is more rigid than the first material.

FIGS. 1A-1B show an exemplary positioning of pulse oximetry system 100 on the subject 1 including a preferred positioning of the cable 108 by the cable retainers 103 and 105. As shown, the dock 106 can be secured to the subject's wrist 3 by the strap 101 such that the cable 108 extends from the dock 106 in a direction generally away from the palm 4 and/or a hand of the subject 1. For example, the pulse oximetry system 100 can be configured such that the cable 108 does not extend from a side of the dock 106 that faces towards the hand of the subject 1 when the dock 106 is secured to the subject's wrist 3. Further to this example, the pulse oximetry system 100 can be configured such that the cable 108 extends from a corner of the dock 106 that faces away from the hand of the subject 1 when the dock 106 is secured to the subject's wrist 3. The cable retainer 103 can be disposed on the strap 101 as shown (e.g., on the strap such that the cable retainer 103 is positioned at a side, such as an outside, of the subject's wrist 3 when the pulse oximetry system 100 is secured to the subject 1) and can secure a portion of the cable 108 between where the cable 108 connects to the dock 106 and where the cable connects to the emitter package 134 comprising the one or more emitters 112 and to the detector package 136 comprising the one or more detectors 114 positioned by the ring member 110. By securing to the cable retainer 103, a bend can be formed in the cable 108 as shown (e.g., the cable 108 is bent towards the subject's palm 4/hand). Such positioning of the cable 108 by the cable retainer 103 can advantageously keep the cable 108 close to the wrist and/or the hand of the subject so as to prevent snagging and/or catching of the cable 108 on external items when the pulse oximetry system 100 is worn by the subject 1. Such positioning of the cable 108 by the cable retainer 103 can also advantageously allow for the length of the cable 108 to be adjusted depending on the length of the hand/palm and finger of the subject (e.g., for a subject with a long hand/palm and pinky finger, the bend formed in the cable may create a loop in the cable that is smaller than a loop created when adjusted for a subject with a relatively shorter hand/palm and pinky finger). In some implementations, the cable 108 can be slidably secured to the cable retainer 103, and as such the bend formed in the cable 108 when secured to the cable retainer 103 can advantageously allow a length of the cable to translate back and/or forth within the cable retainer 103 during motion/movement of the subject's hand about their wrist 3 and/or during movement of the subject's finger 2. The cable retainer 105 can be secured around the subject's finger 2, as shown the subject's pinky finger, between where the ring member 110 is secured to the subject's pinky finger and the subject's palm 4 (more specifically as shown, the cable retainer 105 can be secured around the subject's pinky finger near where the pinky finger connects to the subject's hand, such as around the proximal phalanx). Furthermore, the cable retainer 105 can secure to another portion of the cable 108 between where the cable 108 connects to the dock 106 and where the cable connects to the emitter package 134 comprising the one or more emitters 112 and to the detector package 136 comprising the one or more detectors 114 positioned by the ring member 110. By securing to the cable retainer 105 and the cable retainer 103, the cable 108 can be positioned along an outside edge of the palm 4 of the subject 1 and along at least a portion of an outside edge of the subject's pinky finger. Positioning of the cable 108 along the outside edge of the subject's pinky finger by at least the cable retainer 105 can advantageously allow the subject to freely move, flex, contract, close, etc. their pinky finger without the cable being pulled, bound up, and/or stretched as it would if it were to be located along a top or a bottom of the pinky finger during such movements of the finger. In other words, positioning of the cable 108 along at least a portion of the outside edge of the subject's pinky finger can allow for freedom of movement of the subject's pinky finger without or with little binding and/or pulling of the cable 108 when the pulse oximetry system is worn by the subject. In some implementations, the cable 108 can be slidably secured to the cable retainer 105. As discussed herein, the cable 108 can split into the first portion 107 and the second portion 109 at its end opposite of where the cable 108 connects to the dock 106. As shown and in some implementations, the cable 108 can split into the first portion 107 and the second portion 109 near the ring member 110. In implementations wherein the cable retainer 105 is utilized and secures to both the subject's finger and the cable 108 as discussed herein, the cable 108 can split into the first portion 107 and the second portion 109 between where it is secured to the cable retainer 105 and where it connects, via the first and second portions 107 and 109, to the emitter package 134 comprising the one or more emitters 112 and to the detector package 136 comprising the one or more detectors 114 positioned by the ring member 110, respectively. When the ring member 110 is secured around the subject's pinky finger, the first portion 107 of the cable 108 can extend around the outside edge and along a portion of a top of the subject's pinky finger. Further, when the ring member 110 is secured around the subject's pinky finger, the second portion 109 of the cable 108 can extend around the outside edge and along a portion of a bottom of the subject's pinky finger. In some implementations, cable 108 can be configured such that the split in the cable 108 is positioned adjacent the first joint of the subject's pinky finger when in use, with the first portion 107 extending around the outside edge and along a portion of a top of the intermediate phalanx and with the second portion 109 extending around the outside edge and along a portion of a bottom of the intermediate phalanx. In some implementations, cable 108 can be configured such that the split in the cable 108 is positioned adjacent the second joint of the subject's pinky finger when in use, with the first portion 107 extending around the outside edge and along a portion of a top of the distal phalanx and with the second portion 109 extending around the outside edge and along a portion of a bottom of the distal phalanx. The positioning of the first and second portions 107 and 109 of the cable 108 along portions of the top and bottom of the subject's pinky finger, respectively, can aid in operably positioning the emitter housing 130 and detector housing 132. Further, the ring member 110 can be secured around the subject's pinky finger over the nail bed of the pinky finger. In some implementations, the ring member 110 can be secured near the end of the pinky finger, such as around the distal phalanx or intermediate phalanx.

In some implementations, only one cable retainer, such as the cable retainer 103 or the cable retainer 105, is utilized to position the cable 108 when the pulse oximetry system 100 is worn by and/or secured to the subject 1. In some implementations, no cable retainers are utilized to position the cable 108 when the pulse oximetry system 100 is worn by and/or secured to the subject 1. In some cases, more than two cable retainers can be utilized to position the cable 108 when the pulse oximetry system 100 is worn by and/or secured to the subject 1. Alternatively, or in addition, tape, straps, and/or adhesives can be utilized to position the cable 108 when the pulse oximetry system 100 is worn by and/or secured to the subject 1. The cable 108 (and any portions thereof), can be flexible, semi-flexible, semi-rigid, and/or rigid, and/or a have a mix of flexible, semi-flexible, semi-rigid, and/or rigid portions. In some implementations the cable 108 can have shape memory. The cable 108 can be soft to the touch and/or smooth, which can increase subject comfort when wearing the pulse oximetry system 100.

In some implementations and as shown in FIGS. 1A-1C, 2A-2B, 3, 4A-4D, 5A-5D, 6A-6J, and 8A-8J, the pulse oximetry system 100 and components thereof can include features for enhancing comfort to the subject 1 when the pulse oximetry system 100 is worn by and/or secured to the subject 1. For example, the dock 106 and the sensor hub 102 can have smooth and/or rounded contours where they can contact tissue of the subject. By way of another example and as discussed herein, the ring member 110 and/or the cable retainer 105 can comprise a resilient and/or flexible material.

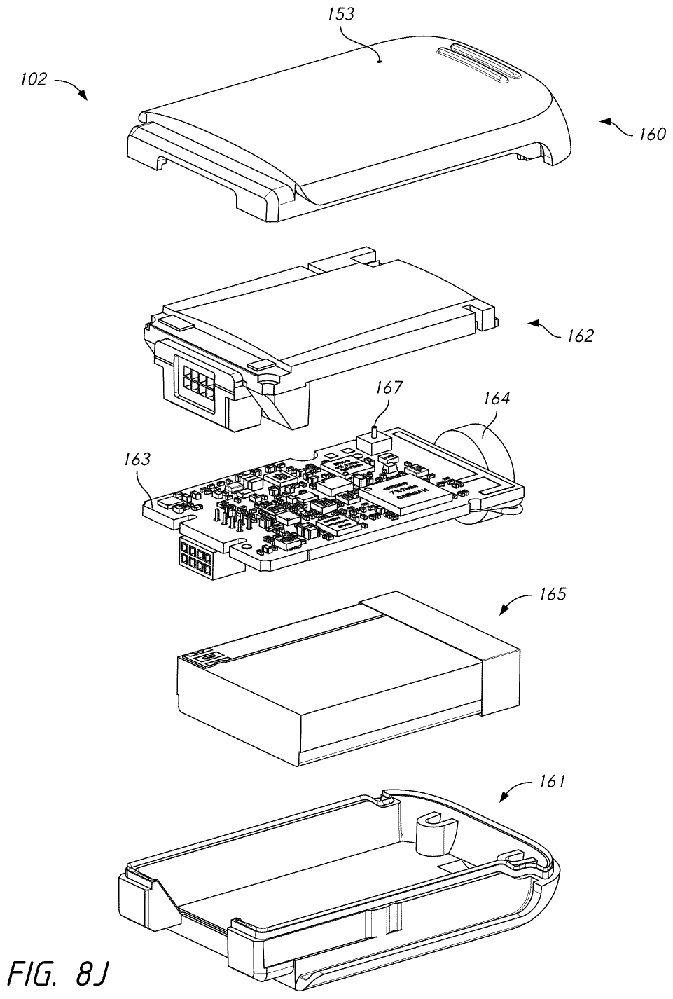

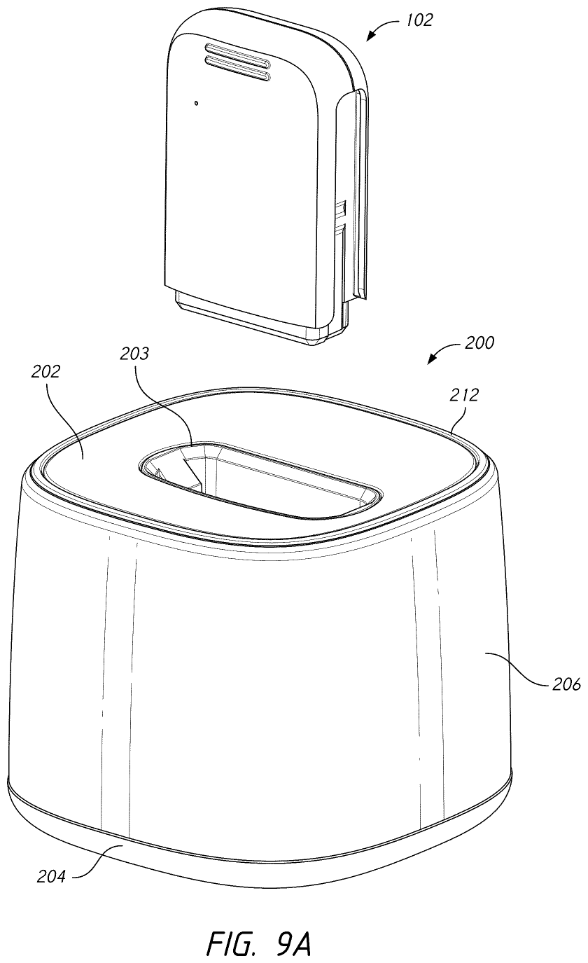

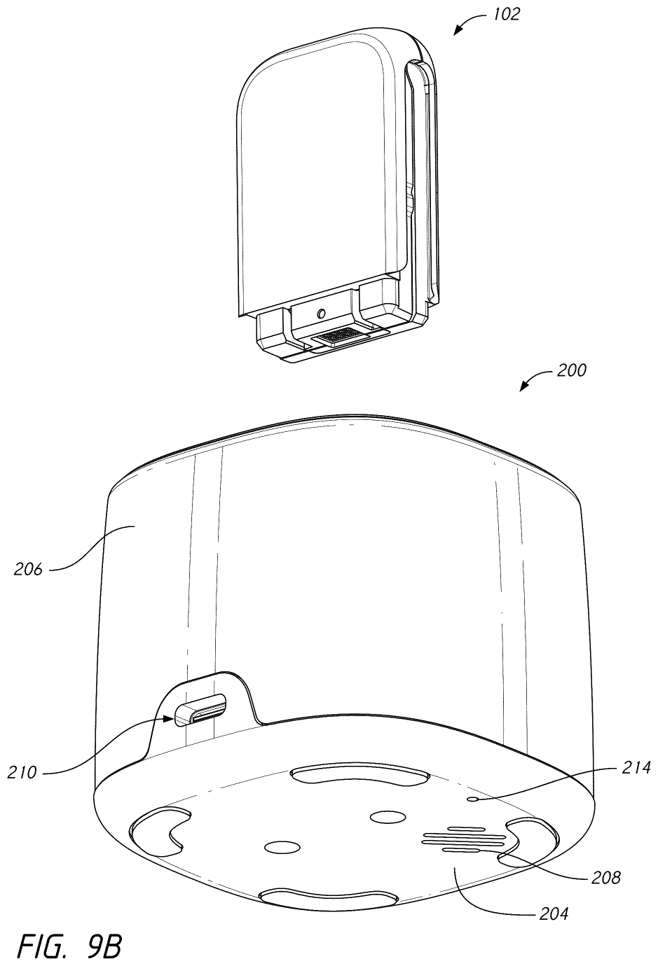

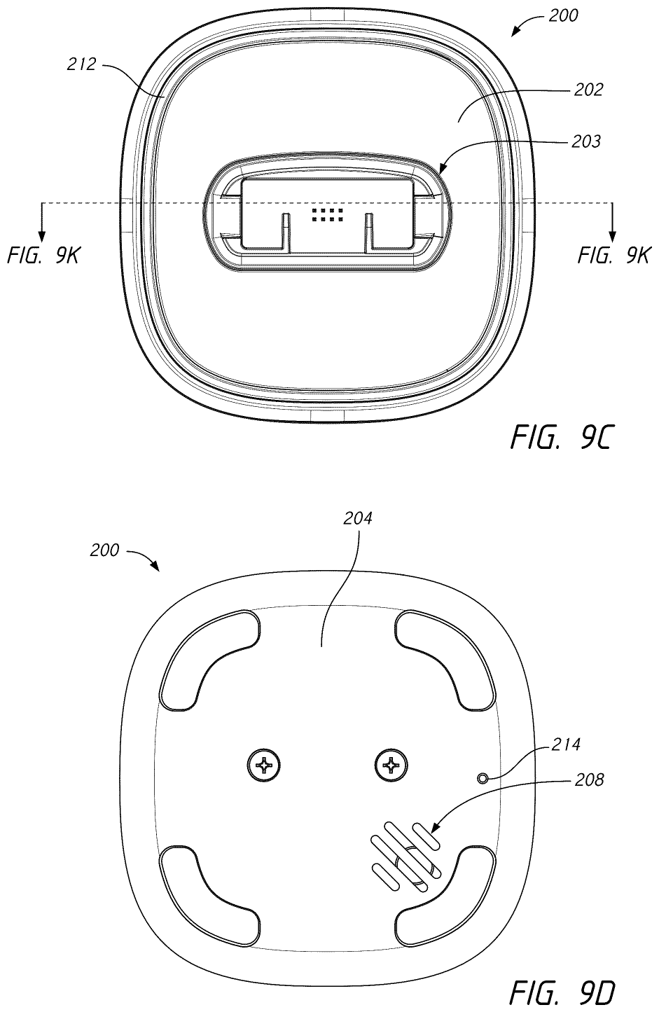

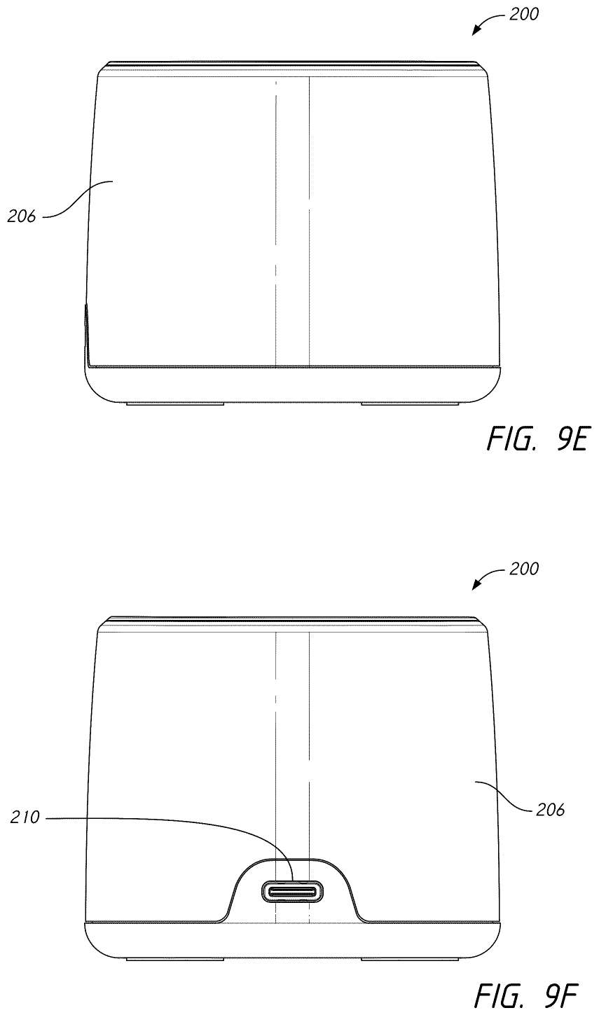

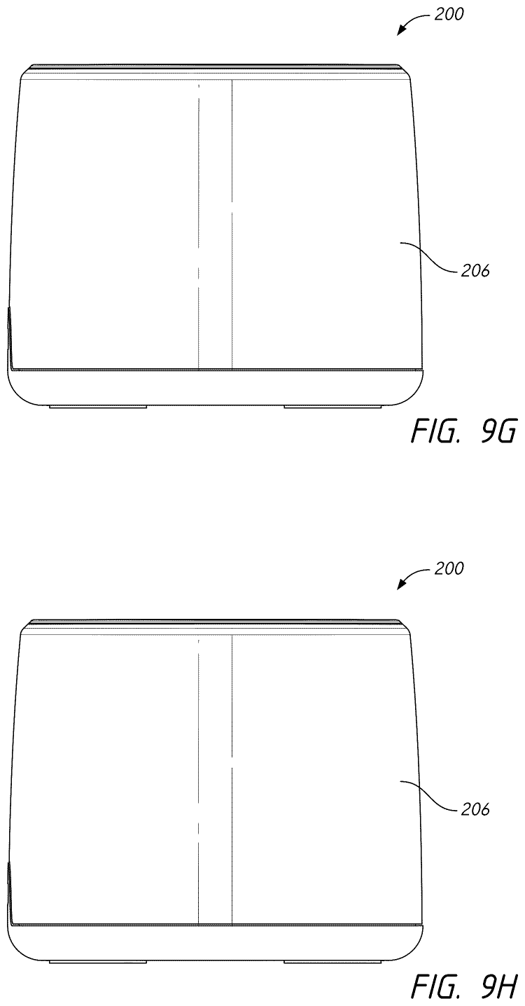

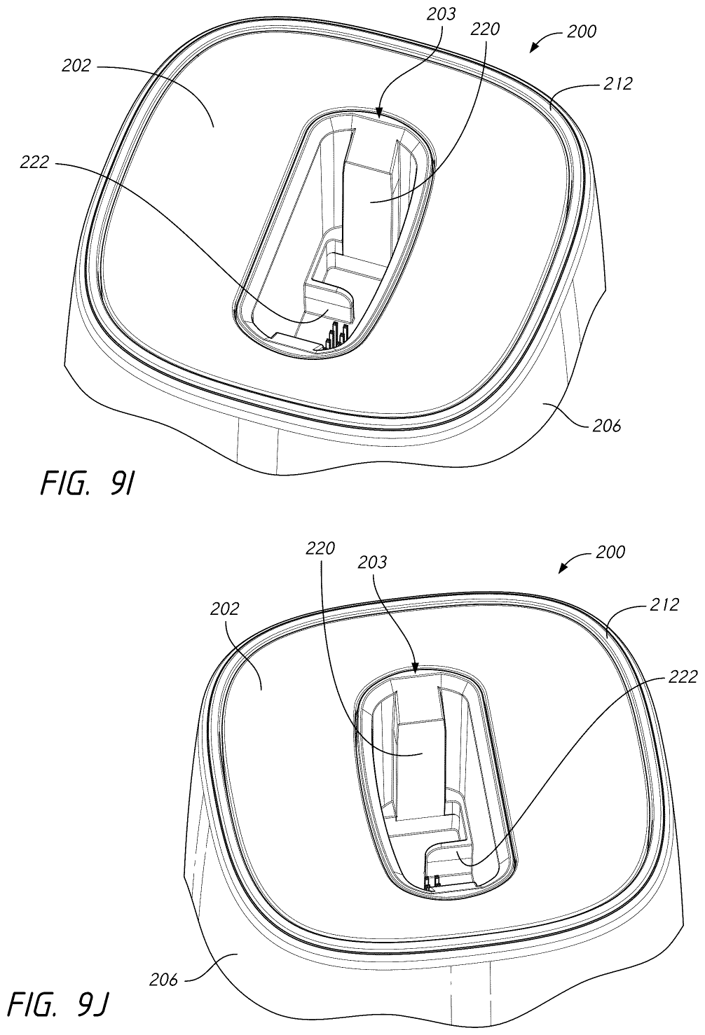

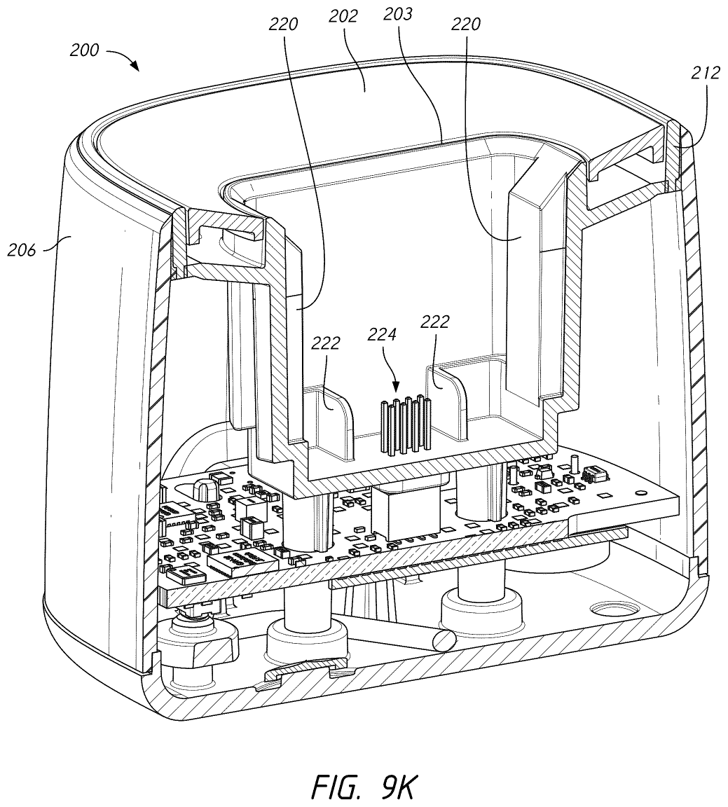

FIGS. 9A-9K illustrate various views of a charging station 200. FIGS. 9A-9B illustrate perspective views of the charging station 200 with the sensor hub 102 described herein removed from the charging station 200, FIG. 9C illustrates a top view of the charging station 200 of FIG. 9A, FIG. 9D illustrates a bottom view of the charging station 200 of FIG. 9A, FIGS. 9E-9F illustrate front and back views, respectively, of the charging station 200 of FIG. 9A, FIGS. 9G-9H illustrate side views of the charging station 200 of FIG. 9A, FIGS. 9I-9J illustrate perspective views of the charging station 200 of FIG. 9A, and FIG. 9K illustrates a perspective cross sectional view through the charging station 200 of FIG. 9A as indicated in FIG. 9C.

The charging station 200 can be configured to releasably mechanically and electrically connect to (e.g., receive) the sensor hub 102. The charging station 200 can, when electrically connected to the sensor hub 102, charge and/or recharge the battery 165 of the sensor hub 102. As shown in FIGS. 9A-9K, the charging station 200 (which can also be referred to herein as a “charging base” and/or “base station”) can comprise a generally cube like body 206 with a bottom plate 204 (which can also be referred to herein as a “bottom surface”), a top plate 202 (which can also be referred to herein as a “top surface”), a cavity 203, and an electrical connector 210. The body 206 of the charging station 200 can have a rounded square like cross section, seen most clearly in the top and bottom views of FIGS. 9C-9D. The charging station 200 can include an indicator 212, opening(s) 208, one or more speakers, a reset button 214, and other features as described further below. Furthermore, the charging station 200 can include any one or more of the features described with respect to the schematic diagram of FIG. 2C, including one or more processor(s) 102a, one or more storage device(s) 102b, a communication module 102c, a battery 102d, an information element 102e, a vibration motor 102f, one or more other sensor(s) 120h, and one or more status indicator(s) 102g.

As shown in FIGS. 9A-9K, the bottom plate 204 can connect to the body 206 and form the bottom portion of the charging station 200. The opening(s) 208 can be disposed on the bottom plate 204 and can, for example, facilitate communication and/or sound from one or more speakers and/or other indicators disposed within the charging station 200 from being transmitted to outside of the charging station 200 (e.g., for a subject to hear and/or be notified). The charging station 200 can be configured to rest on a surface, such as a table top, and as such the bottom plate 204 can include one or more pads, non-slip features, etc. and/or be otherwise configured to provide a stable base for the charging station 200. The bottom plate 204 can also include the electrical connector 210, which can be disposed along a side of the bottom plate 204 such that it is accessible when the charging station 200 rests on/against a surface. As shown, the electrical connector 210 can be disposed along the side of the bottom plate 204 at an end of the charging station 200 that is the back of the charging station 200. In some implementations the electrical connector 210 can instead be disposed on a portion of the body 206, for example the side of the body 206 that is the back end of the charging station 200. The electrical connector 210 can be a connector and/or charging port, such as a USB-C connector/port, that can be configured to provide power to the charging station 200 when operably connected to a power source. In some implementations, the charging station 200 can include a reset button 214 disposed on the bottom plate 204 configured to reset the charging station 200 if pressed and/or pressed and held by the subject.

Further as shown, the top plate 202 can connect to the body 206 and form the top portion of the charging station 200. The top plate 202 can be configured as a push-button, such that the subject can push down on a surface (e.g., the top facing surface) of the top plate 202 to interact with the charging station 200. In some implementations, the surface of the top plate 202 slopes downward towards its center, creating a generally concave surface of the top plate 202. The indicator 212 can be disposed at and/or between the peripheral connection between the top plate 202 and the body 206. For example, the indicator 212 can at least partially circumferentially surround the top plate 202 (as shown, the indicator 212 fully circumferentially surrounds the top plate 202). The indicator 212 can be configured to emit optical radiation and/or allow emission of optical radiation. As an example, the indicator 212 can include one or more emitters configured to emit optical radiation from the charging station 200. As another example, the indicator 212 can be made of a transparent, a semi-transparent, a light transmissible, and/or a partially light transmissible material that can allow optical radiation from one or more emitters located inside the charging station 200 to pass and/or partially pass through. The indicator 212 can be configured to indicate a status of the charging station 200 and/or to indicate a status of the sensor hub 102 when the sensor hub 102 is connected to the charging station 200 (e.g., to indicate a charge state of the battery 165 of the sensor hub 102, such as low charge, medium charge, and/or fully charged).

With continued reference to FIGS. 9A-9K, the cavity 203 of the charging station 200 can be configured to releasably mechanically receive the sensor hub 102. As such, the cavity 203 can be shaped and sized to receive the sensor hub 102. The cavity 203 can be disposed within and/or be defined by an opening in the top plate 202, such that the cavity 203 extends down from the top surface of the charging station 200 towards the bottom of the charging station 200. The cavity 203 can include one or more features for releasably mechanically connecting to the sensor hub 102. For example, the cavity 203 can include stem(s) 220 configured to releasably mechanically connect with the sensor hub 102. The stem(s) 220 can releasably mechanically connect with the sensor hub 102 similar to how the arm(s) 122 of the dock 106 can connect with the sensor hub 102. For example, the stem(s) 220 can be disposed and extend along opposite sides of the cavity 203 and can slidably fit the recessed portion(s) 158 of the sensor hub 102 when the sensor hub 102 is slid into the cavity 203. In some implementations and as shown (e.g., in particular in the top view of FIG. 9C), the cavity 203 can have a variable contour and/or a contour on one side that is different from a contour of an opposite side (e.g., sides that do not have the stem(s) 220) to ensure and/or aid in proper alignment and placement of the sensor hub 102 with the charging station 200.

The cavity 203 can include one or more additional and/or alternative features for releasably electrically and mechanically connecting the sensor hub 102 with the charging station 200. With continued reference to FIGS. 9A-9K, the cavity 203 can include an electrical connector 224 configured to electrically and mechanically connect with the corresponding electrical connector 151 of the sensor hub 102. The sensor hub 102 and its components, such as processor(s) 102a and battery 102d/165, can be operably connected to the charging station 200 and components thereof, including electrical connector 210 for receiving electrical power, when the electrical connector 151 of the sensor hub 102 is connected to the electrical connector 224 of the charging station 200. In some implementations, the electrical connector 151 of the sensor hub 102 can be electrically and mechanically connected to the electrical connector 224 of the charging station 200 when the sensor hub 102 is mechanically connected to the charging station 200, such as by the connection between the stem(s) 220 of the charging station and the recessed portion(s) 158 of the sensor hub 102 as described above. In some cases, the sensor hub 102 can be electrically and mechanically connected to the charging station 200 when it is placed inside the cavity 203. The electrical connector 224 of the charging station 200 can include one or more pins, for example 8 pins as shown in FIGS. 9C and 9K. In some implementations, the electrical connector 224 can include any number of pins. In some implementations, the electrical connector 224 can include one or more openings configured to receive one or more pins of the corresponding electrical connector of the sensor hub 102. As shown in FIGS. 9C and 9K, the electrical connector 224 can be disposed within the cavity 203, for example at the bottom of the cavity 203, such that it faces the sensor hub 102 when the sensor hub 102 is connected to the charging station 200. In some implementations, the charging station 200 can include one or more features to aid in aligning the electrical connector 224 to the corresponding electrical connector of the sensor hub 102. For example, the cavity 203 can include walls 222 adjacent the electrical connector 224, the walls 222 configured to aid in releasably connecting the electrical connector 224 with the corresponding electrical connector 151 of the sensor hub 102.

In some implementations, the charging station 200 includes a communication module that comprises an NFC antenna (for example, within a front side of the body 206) for recognizing and/or communicating with other electronic device(s) and/or sensor(s). In some implementations, the sensor hub 102 can automatically pair with and/or begin electrical communication with the charging station 200 when the sensor hub 102 is mechanically and electrically coupled to the charging station 200, such as by when the sensor hub 102 is seated within cavity 203 of the charging station 200 and the electrical connector 151 of the sensor hub 102 is operably connected with the electrical connector 224 of the charging station 200 (e.g., when the sensor hub 102 is docked with the charging station 200). The charging station 200 can charge the battery 165 of the sensor hub 102 when the sensor hub 102 is docked with the charging station 200. Further, and in some implementations, the sensor hub 102 can download data, such as physiological data from the subject 1, to the charging station 200 and/or to a server, another electronic device, the cloud, and/or a wireless or wired network via the charging station 200. In some implementations, the charging station 200 can update software of the sensor hub 102 when the sensor hub 102 is docked with the charging station 200.

In some implementations (not shown), the charging station 200 can be configured as an array such that it can releasably electrically and mechanically connect to more than one sensor hub 102 at a time. For example, the charging station 200 can be configured to have more than one cavity 203 configured to releasably electrically and mechanically connect to more than one sensor hub 102. In some implementations, the charging station 200 can be configured as a linear array of cavities 203 (e.g., an array of two, three, four, or more cavities). In some cases, the charging station 200 can be configured as an array of cavities 203 with one or more “rows” and one or more “columns.” In some implementations, the charging station 200 can be configured to slidably receive the sensor hub 102 in a vertical orientation, such as shown in FIG. 9A. In some implementations, the charging station 200 can be configured to slidably receive the sensor hub 102 in an orientation other than vertical, such as at an angle to the vertical, sideways, horizontally, etc.

In some implementations, the charging station 200 can include a battery 102d configured as a backup battery for providing power/charge to a sensor hub 102 even if the power source that provides power to the charging station 200 is unavailable. Such a backup battery can be sized/rated and/or have a capacity to provide a partial charge, a full charge, two full charges, more than two full charges or any amount of a partial or a full charge to the sensor hub 102 in the case of a power outage.

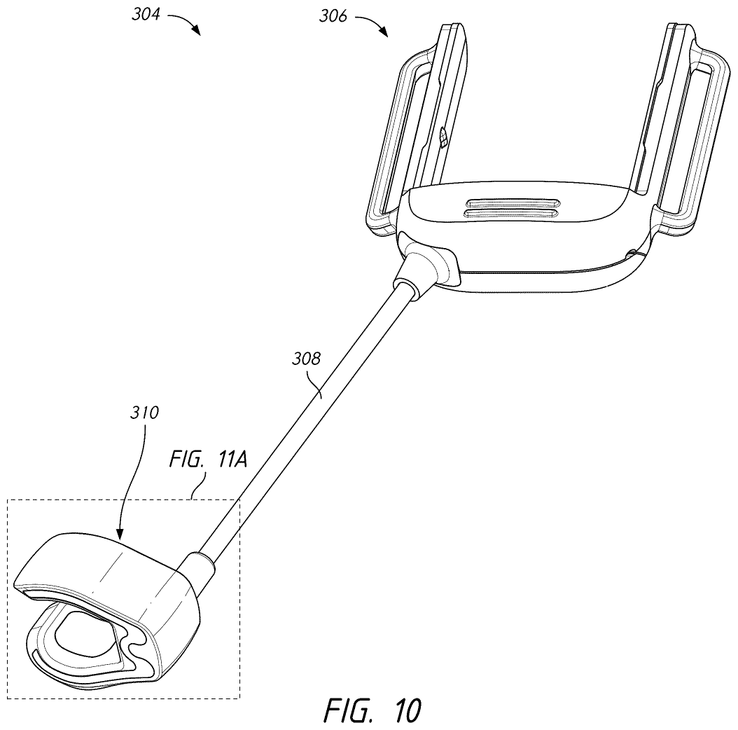

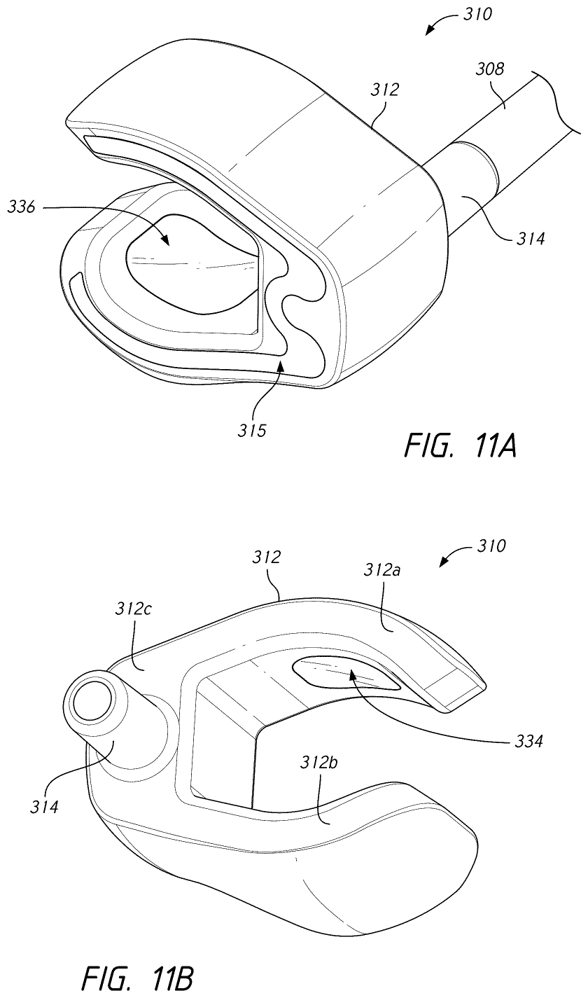

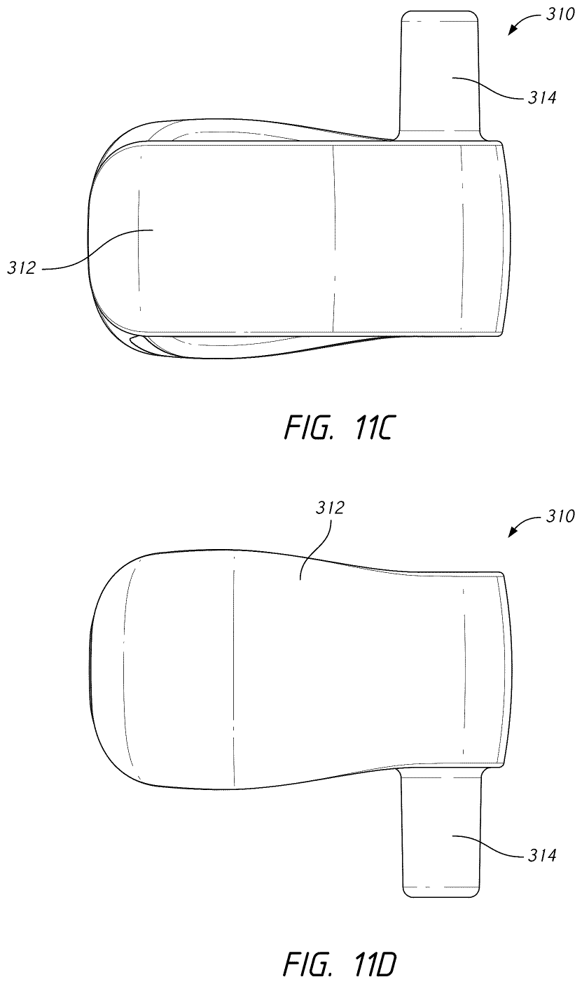

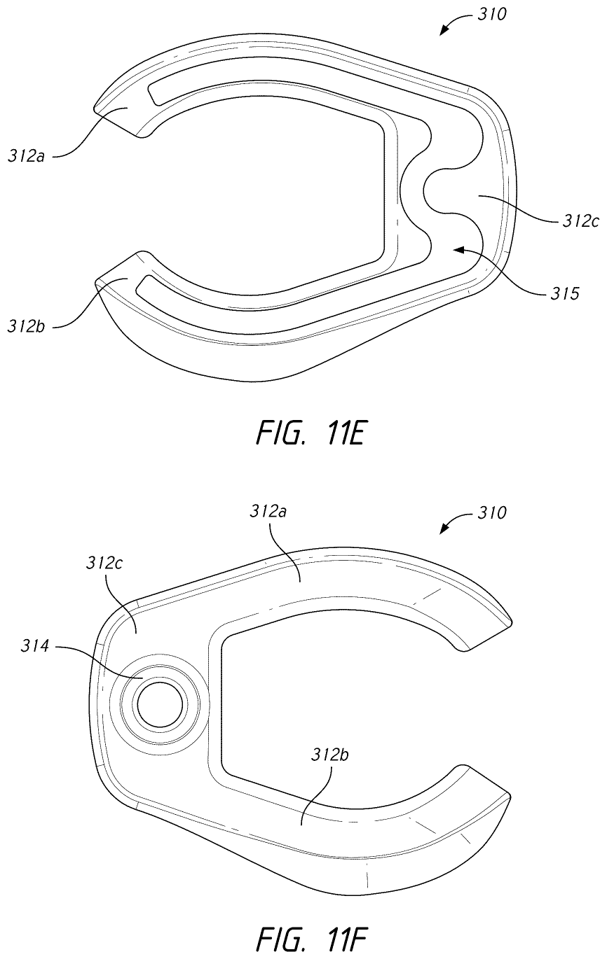

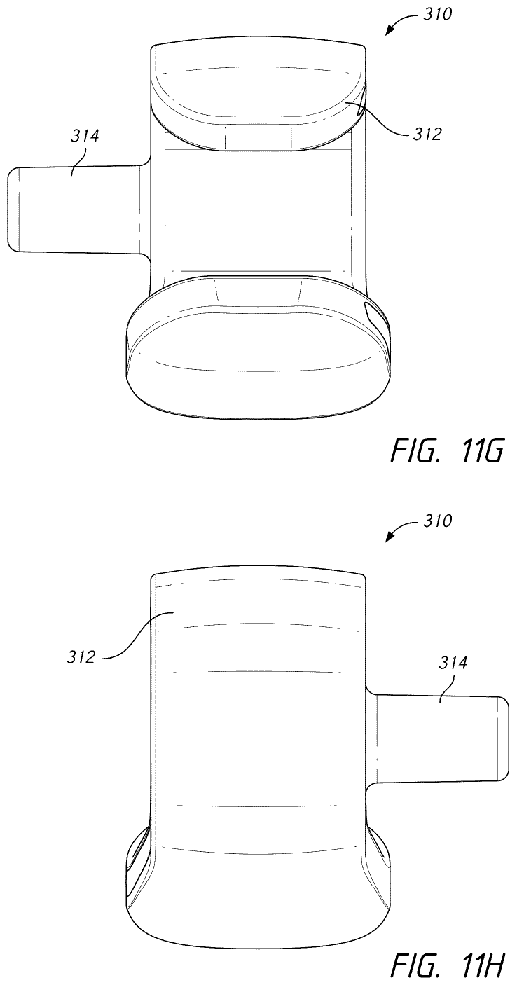

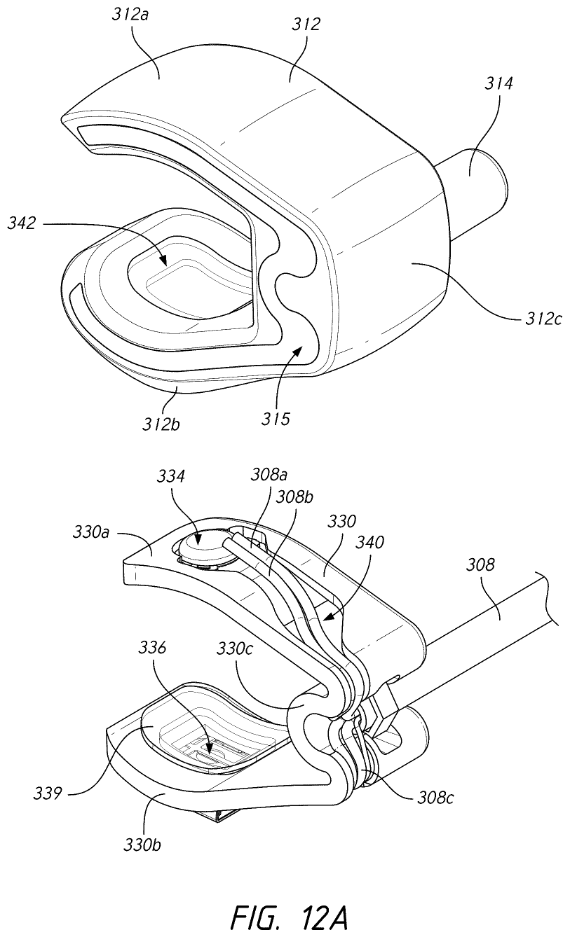

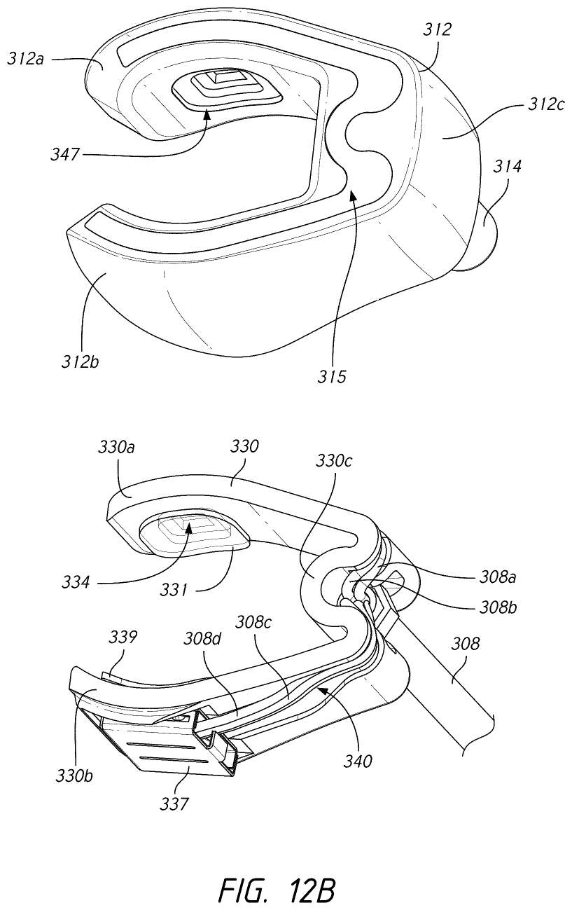

FIG. 10 illustrates a perspective view of another implementation of a sensor dock assembly 304 that can be utilized with sensor hub 102 in a similar or identical manner as that described above with respect to sensor dock assembly 104. The sensor dock assembly 304 can be similar or identical to the sensor dock assembly 104 in some or many respects. For example, the sensor dock assembly 304 can have a sensor dock 306 that is similar or identical to the sensor dock 106 of the sensor dock assembly 104. The sensor dock assembly 304, in particular the dock 306, can thus releasably electrically and mechanically connect with the sensor hub 102 similar and/or the same as how the sensor hub 102 releasably electrically and mechanically connects to the sensor dock assembly 104 via the dock 106. The sensor dock assembly 304 can also be configured to be secured to the wrist 3 of the subject 1 similar and/or the same as how the sensor dock assembly 104 can be secured to the wrist 3 of the subject 1. Further, the sensor dock assembly 304 can have a cable 308 configured similar and/or the same as the cable 108 of the sensor dock assembly 104. The cable 308 can operably connect the dock 306 with one or more emitters of an emitter package 334 and one or more detectors of a detector package 336. The emitter package 334 and the detector package 336 can be the same and/or similar to emitter package 134 and detector package 336 described herein. While not shown, the cable 308 can be secured to and/or positioned along the subject 1 similar and/or the same as how the cable 108 can be secured to and/or positioned along the subject 1 when the sensor dock assembly 304 is secured to the subject 1, including in some implementations the use of one or more cable retainers which can be the same and/or similar to cable retainers 103, 105 described herein. For example, the dock 306 can be secured to the subject's wrist 3 by a strap (not shown) such that the cable 308 extends from the dock 306 in a direction generally away from the palm 4 and/or the hand of the subject 1, the cable 308 can be secured by one or more cable retainers (e.g., the same or similar to cable retainer 103 described herein) to cause the cable 308 to bend towards the palm 4 and/or the hand of the subject 1 and extend along the outside edge of the subject's palm 4 towards the subject's pinky finger, the cable 308 can be secured by one or more cable retainers (e.g., the same or similar to cable retainer 105 described herein) to aid in causing the cable 308 to extend along the outside edge of the palm 4 and to cause the cable 308 to extend along at least a portion of the outside edge of the subject's pinky finger until the cable 308 connects with the emitter package 334 and the detector package 336 operably positioned by a clip member 310. The sensor dock assembly 304 can differ from the sensor dock assembly 104 in that instead of having the ring member 110, the sensor dock assembly 304 can have the clip member 310 for operably positioning the emitter package 334 and the detector package 336. In some implementations and as shown, the cable 308 can differ from the cable 108 in that it does not split into a first portion and a second portion but instead remains as a singular cable that connects with the clip member 310. Other aspects of the sensor dock assembly 304 will be described further below with respect to FIGS. 11A-11H and FIGS. 12A-12B.

FIGS. 11A-11B illustrate perspective views of the clip member 310 and a portion of the cable 308 connecting to the clip member 310. Also shown are positions of the emitter package 334 and the detector package 336 with respect to the clip member 310. FIG. 11C illustrates a top view, FIG. 11D illustrates a bottom view, FIGS. 11E-11F illustrate front and back views, respectively, and FIGS. 11G-11H illustrate side views of the clip member 310. FIGS. 12A-12B illustrate perspective views of the clip member 310 with a housing 312 of the clip member 310 separated from a biasing member 330 of the clip member 310, the emitter package 334, and the detector package 336.

The clip member 310 can be configured to be secured around the finger 2 of the subject 1. In some implementations, the clip member 310 can be secured around the finger 2 of the subject 1 similar and/or the same as how the ring member 110 is secured around the finger 2 of the subject 1 (e.g., over a nail bed of the subject's pinky finger). For this, the housing 312 of the clip member 310 can be in a generally C shaped and/or a partial ring configuration with one or more prongs configured to secure around at least a portion of the subject's finger 2. For example, the housing 312 of the clip member 310 can include a prong 312a and a prong 312b configured to secure around opposite sides of the subject's finger 2. Prongs 312a, 312b can be coupled to one another, such as is shown. Prongs 312a, 312b can be configured to flex toward and/or away from one another, for example, during securement to the subject's finger 2. Prongs 312a, 312b can be configured to move (for example, flex) between various positions with respect to one another. For example, prongs 312a, 312b can be configured to flex from and/or between a first position and/to a second position. Such first position can be a neutral and/or unstressed position and/or state of the prongs 312a, 312b. Such first position can be a position and/or state where the prongs 312a, 312b are not secured to a subject's finger 2. Such a second position can be a stressed position and/or state of the prongs 312a, 312b where the prongs 312a, 312b are at least partially flexed (for example, flexed open). For example, such second position can be a position or state when the clip member 310 is secured to the subject's finger 2. Prongs 312a, 312b or portions thereof (for example, free ends thereof) can be positioned closer to one another when in such first position than when in such second position.

With reference to at least FIGS. 11E-11F, clip member 210 can include a coupling portion 312c. Coupling portion 312c can connect prongs 312a, 312b (for example, ends of prongs 312a, 312b) and/or can be positioned between prongs 312a, 312b (for example, between ends of prongs 312a, 312b). As shown, coupling portion 106 can be generally straight, however it can be curved, straight, or a combination thereof. Coupling portion 312c can be sized and/or shaped to conform to a size and/or shape of the outside edge of the subject's finger 2. In some implementations, coupling portion 312c can be integral and/or contiguous with one or both of prongs 312a, 312b. For example, the housing 312 of the clip member 310 can at least partially define and/or form the coupling portion 312c and prongs 312a, 312b. In some implementations, the entire portions of the housing 312 of the clip member 310 that contacts and/or is placed proximate or adjacent the subject's finger 2 when in use can be contiguous, which can greatly increase comfort to the subject. In some implementations, entire exterior portions of the prongs 312a, 312b and the coupling portion 312c can be defined by a single contiguous housing 312. In some implementations, such housing 312 can comprise a resilient and flexible material (e.g., a silicone material). In some cases, the housing 312 of the clip member 310 can be made of a material similar to and/or the same as the ring member 110.

With continued reference to FIG. 10, FIGS. 11A-11H, and FIGS. 12A-12B, the clip member 310 can include a bend relief 314 where coupled to the cable 308. The bend relief 314 can be integral with and/or contiguous with one or both of prongs 312a, 312b and/or coupling portion 312c. As such, the bend relief 314 can be integrated with the housing 312. The bend relief 314 can extend outward from the clip member 310 in a direction along the outside edge of the finger of the subject 1 when the clip member 310 and the rest of the sensor dock assembly 304 is secured to the subject. Additionally, the bend relief 314 can be configured to extend toward the wrist 3 of the subject 1 when the clip member 310 is secured around the subject's finger 2 (e.g., the subject's pinky finger). As shown, the bend relief 314 can extend in a direction different than the prongs 312a, 312b (e.g., in a direction orthogonal to the prongs 312a, 312b). The bend relief 314 can be configured to receive the cable 308. For example, the bend relief 314 can include an opening sized and/or shaped to receive and/or secure the cable 308.

In some implementations and as shown in FIGS. 11E-11F, prongs 312a, 312b can each have a generally arcuate shape for securing around opposite sides of the subject's finger 2 (e.g., the prongs 312a, 312b can each comprise a bend and/or a curve for conforming to the top and bottom of the subject's finger, respectively). Further as shown, the prongs 312a, 312b can be generally of the same or similar length. The width of the prongs 312a, 312b can be different as shown in FIGS. 11G-11H, however in some implementations the width of the prongs 312a, 312b can be generally similar or the same. Such configuration can ensure that the clip member 310 comfortably contacts a portion of the subject's finger 2 in a manner which provides a robust skin-sensor interface, which advantageously provides for more accurate measurement of physiological parameters via the emitters package 334 and the detector package 336.

As discussed above, the clip member 310 can include a housing 312 that can at least partially define and/or form the coupling portion 312c, prongs 312a, 312b, and/or bend relief 314. With reference to FIGS. 12A-12B, the housing 312 can include an opening 315, which can be located in and extend at least part way through and across the prongs 312a, 312b and the coupling portion 312c. As shown, the opening 315 opens into an end of the housing 312 (e.g., the front) opposite an end where the bend relief 314 extends from (e.g., the back). The housing 312 can also include a cavity 342 located in prong 312b and/or a cavity 347 located in prong 312a. The cavities 342 and 347 can join and/or connect with the opening 315. The cavities 342 and 347 can extend through the prongs 312b and 312a, respectively, such that they open through the housing 312 in directions where they generally face each other. Together, opening 315 and cavities 342, 347 can be sized and shaped to receive the biasing member 330, the emitter package 334, optical transmission material 331 (which can be the same or similar to optical transmission material 131 described herein), the detector package 336, optical transmission material 339 (which can be the same or similar to optical transmission material 139 described herein), detector shield 337 (which can be the same or similar to detector shield 137 described herein), wires 308a and 308b that extend from the cable 308 and operably connect to the one or more emitters within the emitter package 334, and/or wires 308c and 308d that extend from the cable 308 and operably connect to the one or more detectors within the detector package 336. Specifically, opening 315 and cavity 342 can be sized and shaped to receive the biasing member 330, the detector package 336, optical transmission material 339, detector shield 337, and wires 308c, 308d, and opening 315 and cavity 347 can be sized and shaped to receive the biasing member 330, the emitter package 334, optical transmission material 331, and wires 308a, 308b.

In some implementations and as shown, the biasing member 330 can include a recess 340 configured to receive wires 308a, 308b, 308c, 308d, the emitter package 334 or a portion thereof, the detector package 336 or a portion thereof, and/or the detector shield 337 or a portion thereof. The recess 340 can allow for components or portions thereof of the clip member 310 (such as those just described) to fit recessed below an outer surface of the biasing member 330, which can facilitate assembly of the housing 312 over the biasing member 330 and other components shown. In some cases, the clip member 310 can be assembled by inserting the biasing member 330, the wires 308a, 308b, 308c, 308d, the emitter package 334, the detector package 336, optical transmission material 331, optical transmission material 339, and/or detector shield 337 into the openings 315 and cavities 342, 347 of housing 312, and subsequent to such placement, enclosing the opening 315. For example, the opening 315 can be sealed or filled in with a material (such as silicone or another material that forms part of the housing 312). In some implementations, the opening 315 can be sealed (for example, during manufacturing of sensor dock assembly 304) with a sealant (such as a liquid sealant) which can cover the biasing member 330. Such a sealant and/or filler can be exposed and/or form at least a portion of an exterior of the clip member 310, for example, along with the housing 312. In some cases, such a sealant can cover the opening 315 and the housing 312 to form the entire exterior of the clip member 310 that is visible.

As discussed above, the clip member 310 can include the biasing member 330. With reference to FIGS. 12A-12B, the biasing member 330 can include an arm 330a and an arm 330b spaced apart from one another and extending out in a similar direction from a coupling portion 330c. In some implementations and as shown, the biasing member 330 can have a generally W shaped cross section (although in other implementations, the coupling portion can be configured differently and the biasing member 330 can have a generally C shaped cross section or any other cross sectional shape). As shown, the arms 330a, 330b and coupling portion 330c of the biasing member 330 can comprise a combination of straight and/or curved portions and can be configured to aid in the clip member 310 being secured around a portion of the subject's finger 2 when the housing 312 is assembled over the biasing member 330. When the clip member 310 is assembled, the arm 330a of the biasing member 330 can be enclosed by the prong 312a of the housing 312, the arm 330b of the biasing member 330 can be enclosed by the prong 312b of the housing 312, and the coupling portion 330c of the biasing member 330 can enclosed by the coupling portion 312c of the housing 312. The biasing member 330 can be flexible and/or resilient. The biasing member 330 can comprise plastic, among other materials. The biasing member 330 can comprise a harder and/or more rigid material than housing 312. The biasing member 330 can be configured to have a greater stiffness than housing 312. The biasing member 330 can be configured to allow the clip member 310 to move (for example, flex) between a variety of different positions, such as the first and second positions discussed above, to facilitate positioning and/or securement of the clip member 310 to a portion of the subject's body (for example, the subject's pinky finger). The arms 330a, 330b of the biasing member 330 can be configured to flex and move away from and/or toward each other. Biasing member 330 can “bias” the clip member 310 to the first position (for example, neutral and/or unstressed position as discussed above). Further, the biasing member 330 can provide the clip member 310 with a biasing and/or spring-like force to allow the clip member 310 to secure to the finger 2 of the subject 1 (for example, when in the second position). In some implementations, the coupling portion 330c can advantageously define a flex point or region of the biasing member 330. For example, biasing member 330 can be configured to flex about and/or with respect to the coupling portion 330c. In some cases, such configuration can allow the arms 330a, 330b to flex more easily, which can advantageously allow for better positioning and placement of the emitter package 334 and the detector package 336 of the sensor dock assembly 304 with respect to the subject's finger 2. In some cases, the coupling portion 330c can provide mechanical support for the wires 308a, 308b, 308c, 308d, for example, portions of such wires 308a, 308b, 308c, 308d that are bent and/or otherwise oriented at an angle. In some implementations, the coupling portion 330c can advantageously add stiffness and/or rigidity to the biasing member 330. In some configurations, the biasing member 330 can provide stiffness, rigidity, and/or structure to the clip member 310.

When the clip member 310 is assembled, biasing member 330 can be positioned proximate and/or can operably position the one or more emitters in emitter package 334, the one or more detectors in detector package 336, the optical transmission material 331, the optical transmission material 339, and/or the detector shield 337. Biasing member 120 can advantageously include one or more openings configured to allow optical radiation emitted from the emitter package 334 to pass therethrough and allow at least a portion of the optical radiation attenuated by the subject's tissue to pass to the detector package 336. For example, as shown in FIGS. 12A-12B, the biasing member 330 can include an opening extending through a portion of arm 330a and an opening extending through a portion of arm 330b, both of which can be positioned at or near an end of the arms 330a, 330b (for example, free ends of arms 330a, 330b). The openings can be at least partially aligned with one another and/or can at least partially face one another. When the biasing member 330 and/or the clip member 310 is in the first position (discussed elsewhere herein), the openings can be at least partially aligned with one another and/or can at least partially face one another. Additionally or alternatively, when the biasing member 330 and/or the clip member 310 is in the second position (discussed elsewhere herein), the openings can be at least partially aligned with one another and/or can at least partially face one another.

In some implementations, the housing 312 and/or the biasing member 330 can be configured to inhibit, prevent, and/or reduce an amount of ambient light, stray light, and/or any optical radiation not emitted from the emitter package 334 from reaching the detector package 336. Additionally, or alternatively, the housing 312 and/or the biasing member 330 can be configured to inhibit, prevent, and/or reduce an amount of optical radiation emitted by the emitter package 334 that has not been attenuated by, reflected by, and/or passed through tissue of the subject from being received by the detector package 336. In some cases, the housing 312 and/or the biasing member 330 can be opaque and/or generally light blocking and/or have a light blocking coating. In some implementations, the housing 312 and/or the biasing member 330 can be semi-transparent or transparent. In some implementations, the housing 312 and/or the biasing member 330 can include portions that are opaque and/or light blocking and portions that are semi-transparent and/or transparent.

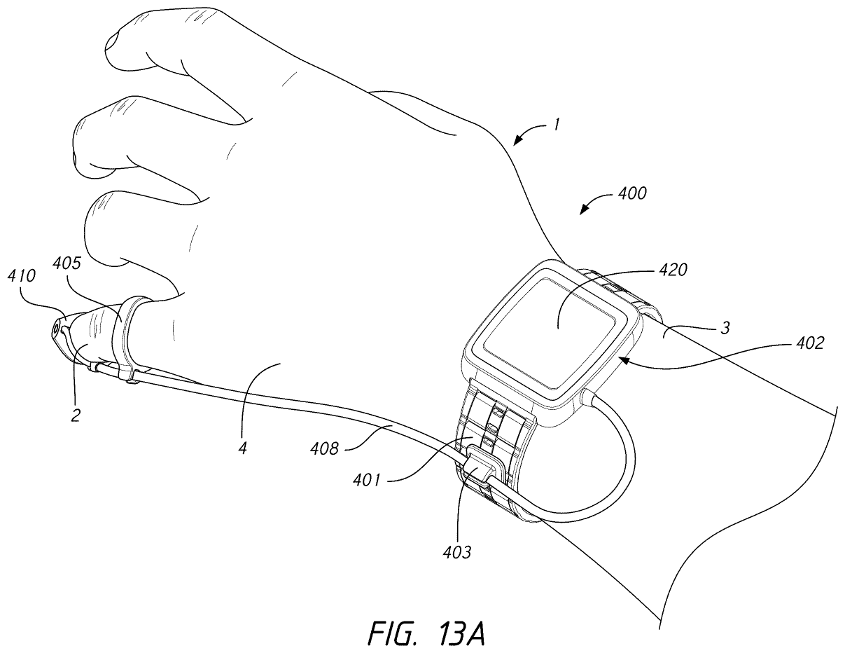

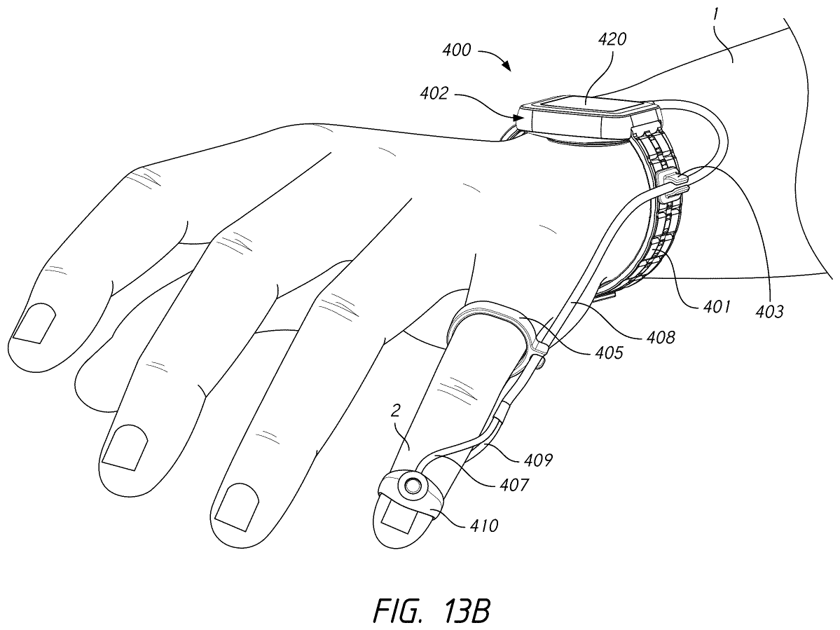

FIGS. 13A-13B illustrate perspective views of another implementation of a pulse oximetry system 400 in the form of a watch secured to a subject's wrist 3 and finger 2 in accordance with some aspects of this disclosure. The pulse oximetry system 400 can have similar and/or the same features, aspects, and/or components as the pulse oximetry system 100 described herein. For example, the pulse oximetry system 400 can have a watch 402 configured similar to the sensor dock 106 and the sensor hub 102 (e.g., the watch 402 can include and/or combine any or all aspects of the sensor dock 106 and the sensor hub 102). The watch 402 can be configured to be secured to the wrist 3 of the subject 1 similar and/or the same as how the sensor dock 106 can be secured to the wrist 3 of the subject 1, for example, by a strap 401 similar and/or the same as the strap 101. Further, the watch 402 can have a cable 408 configured similar and/or the same as the cable 108 of the sensor dock assembly 104, including a split into a first portion 407 and a second portion 409 similar and/or the same as the first portion 107 and the second portion 109, respectively. The cable 408 can operably connect the watch 402 with an emitter package comprising one or more emitters (similar and/or identical to the those described herein, such as emitter package 134 comprising one or more emitters 112) and a detector package comprising one or more detectors (similar and/or identical to the those described herein, such as detector package 136 comprising one or more detectors 114) of the pulse oximetry system 400. In some implementations, the cable 408 can be detachable from the watch 402. For example, in some implementations, the cable 408 can be removably connectable to a connector port on a portion of watch 402. The cable 408 can be secured to and/or positioned along the subject 1 similar and/or the same as how the cable 108 can be secured to and/or positioned along the subject 1 when the pulse oximetry system 400 is secured to the subject 1, including in some implementations the use of one or more cable retainers 403 and/or 405 which can be the same and/or similar to the cable retainers 103 and 105 described herein. For example, the watch 402 can be secured to the subject's wrist 3 by the strap 401 such that the cable 408 extends from the watch 402 in a direction generally away from the palm 4 and/or the hand of the subject 1, the cable 408 can be secured by the cable retainer 403 (e.g., the same or similar to cable retainer 103 described herein, which can be adhered to the strap 401 as shown) to cause the cable 408 to bend towards the palm 4 and/or the hand of the subject 1 and extend along the outside edge of the subject's palm 4 towards the subject's pinky finger, the cable 408 can be secured by the cable retainer 405 (e.g., the same or similar to cable retainer 105 described herein) to aid in causing the cable 408 to extend along the outside edge of the palm 4 and to cause the cable 408 to extend along at least a portion of the outside edge of the subject's pinky finger until the cable 408 splits into the portion 407 and the portion 409 to connect with the emitter package and the detector package operably positioned by a ring member 410, respectively. The ring member 410 can be the same and/or similar to the ring member 110 described herein. Cable retainer 403 can be permanently or removably attached to one of straps 401. For example, in some implementations, cable retainer 403 is configured to removably attach to a portion of one or both of straps 401 via a snap-fit securement. As another example, in some implementations, cable retainer 403 is configured to removably attach to a portion of one or both of straps 401 via an adhesive securement.

The watch 402 (including the strap 401) of the pulse oximetry system 400 can be similar or identical to and/or incorporate any of the features described with respect to any of the devices, assemblies, and/or systems described and/or illustrated in U.S. Pat. Pub. No. US2021/0290120, filed Jan. 13, 2021, titled “WEARABLE DEVICE WITH PHYSIOLOGICAL PARAMETERS MONITORING,” which is hereby incorporated by reference in its entirety and for all purposes. For example, the watch 402 can include a display 420 similar or identical to the display of the wearable sensor described in U.S. Pat. Pub. No. US2021/0290120 incorporated by reference herein, which can be configured to display a plurality of physiological parameters monitored and/or determined by the pulse oximetry system 400 and/or other information (e.g., such as time, date, etc.). Further, the display 420 can be configured to receive input from the subject, such as touch input, for interacting with the pulse oximetry system 400. For example, the display 420 can be a capacitive or resistive touch screen for receiving user input.

In some implementations, the watch 402 can be powered by a battery. The battery can be rechargeable. The battery can be recharged by connecting the watch 402 to a source of electrical power (e.g., via a cable), by wirelessly charging (e.g., via a wireless charging pad), by solar energy (e.g., via a solar collector if incorporated in the watch 402), and/or by kinetic motion (e.g., via an internal mechanism if incorporated that can convert kinetic motion into electrical power). In some cases, the battery can be removed, or the battery can be integrated within and/or a permanent part of the watch 402. In some implementations, the watch 402 includes a connector port for connecting to a charging cable. Such a connector port can additionally be used to connect to cable 408 in some implementations.

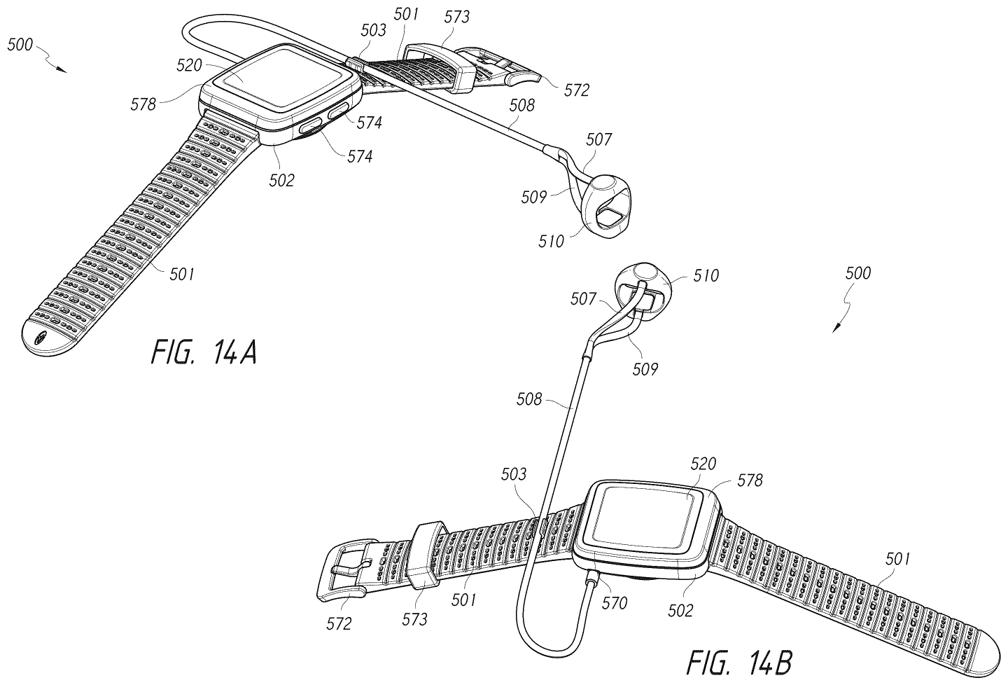

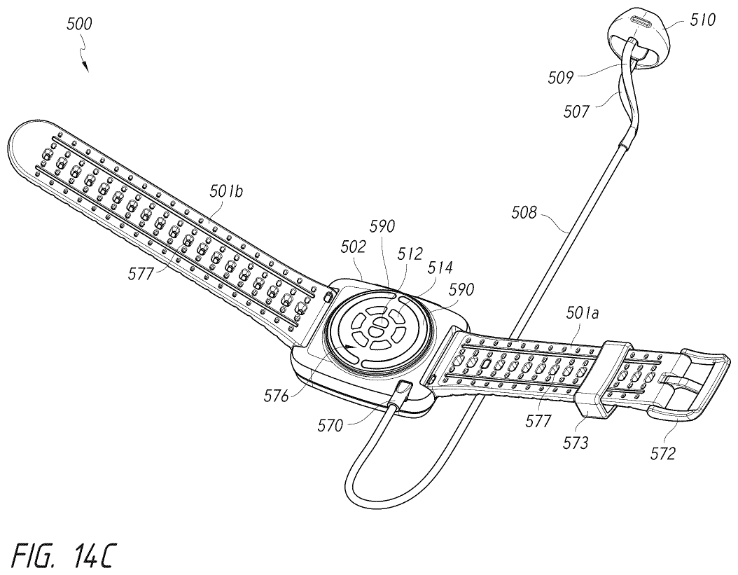

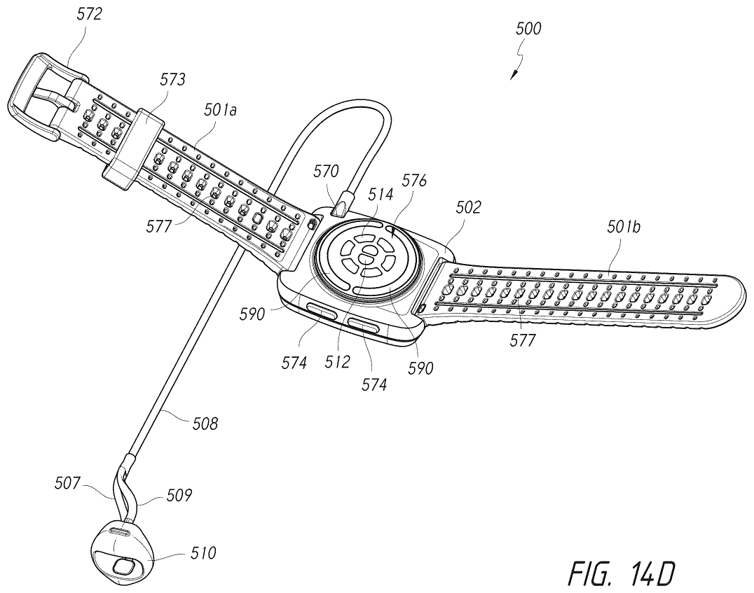

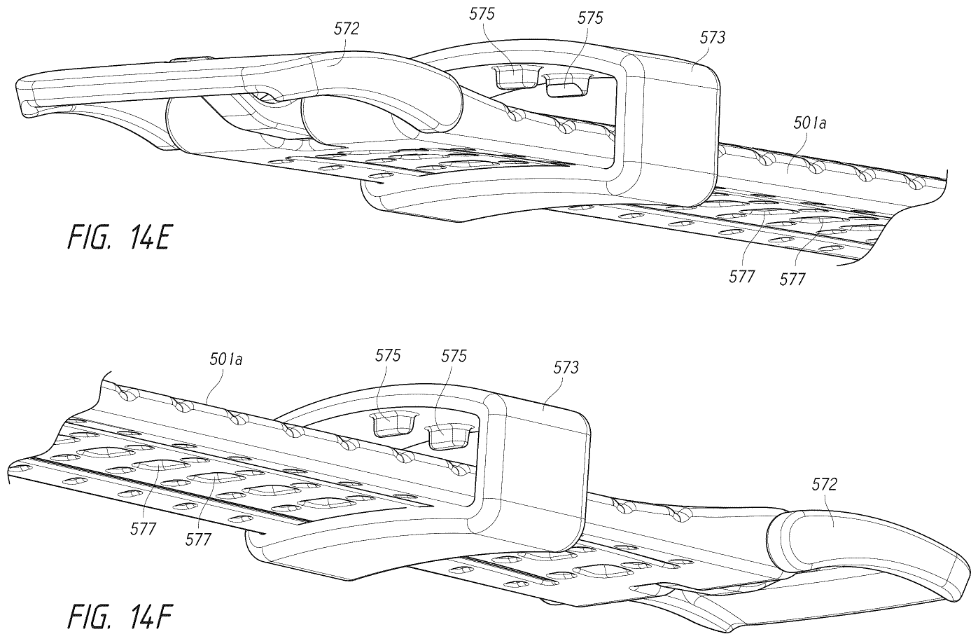

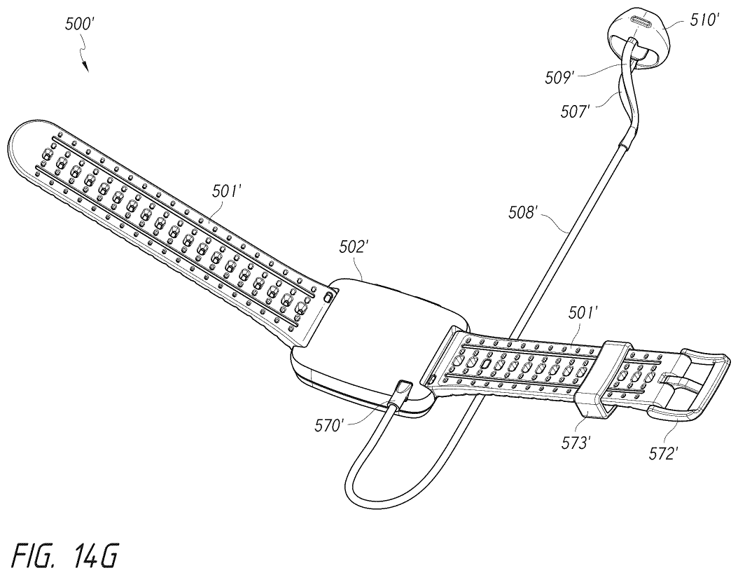

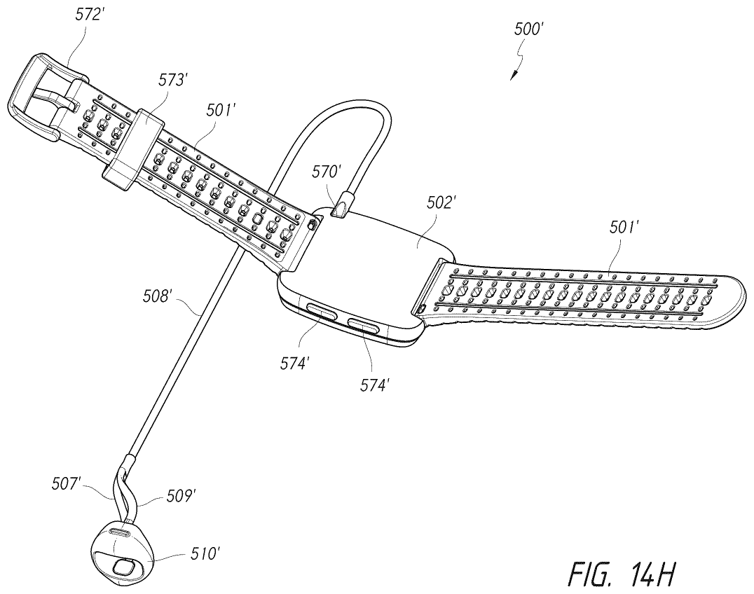

FIGS. 14A-14B illustrate top perspective views and FIGS. 14C-14D illustrate bottom perspective views of another implementation of a pulse oximetry system 500. Pulse oximetry system 500 includes a watch 502 configured to be secured to a subject's wrist via straps 501. Pulse oximetry system 500 also includes a cable 508 coupled to a ring member 510. Cable 508 can be similar or identical to cable 108 discussed above. Ring member 510 can be similar or identical to any of the ring members described herein, such as ring member 110. In some implementations, cable 508 splits into a first portion 507 and a second portion 509 in a manner similar or identical to that discussed above with respect to cable 108, portion 107, and portion 109. Similar to that discussed with respect to cable 108, cable 508 can operably connect watch 502 with an emitter housing and/or emitter package comprising one or more emitters (similar and/or identical to the those described herein, such as emitter package 134 comprising one or more emitters 112) and a detector housing and/or a detector package comprising one or more detectors (similar and/or identical to the those described herein, such as detector package 136 comprising one or more detectors 114). In some implementations, cable 508 can be detachable from the watch 502, for example, removably connectable to a connector port 570 of watch 502 (see FIG. 14B-14D). Alternatively, in some implementations, cable 508 is permanently attached to watch 502. The cable 508 can be secured to and/or positioned along a subject similar or identical to that discussed with respect to cable 108 in pulse oximetry system 100, for example, with the utilization of one or more cable retainers (such as cable retainers 103, 105) and/or similar to that discussed above with respect to pulse oximetry system 100. In some implementations, pulse oximetry system 500 includes a cable retainer 503 that can be similar or identical to any of the cable retainers discussed herein, such as cable retainer 403. Cable retainer 503 can be permanently or removably attached to one of straps 501. A buckle 572 (for example, having a buckle tongue) can be coupled to one of the straps 501 to allow the straps 501 to be secured to one another (for example, around a subject's wrist), although the straps 501 can be secured to one another in other ways (e.g., via magnets, via the straps having a shape memory, or the like). In some implementations, the watch 502 can receive and/or process one or more signals outputted by one or more detectors operably positioned by ring member 510 and determine one or more physiological parameters based on the received or processed signals. In some implementations, watch 502 can instruct one or more emitters operably positioned by ring member 510 to emit light of one or more wavelengths, for example, in tissue of a finger to which ring member 510 is secured during use.

In some implementations, the watch 502 can be powered by a battery. The battery can be rechargeable. The battery can be recharged by connecting the watch 502 to a source of electrical power (e.g., via a cable that can connect to connector port 570), by wirelessly charging (e.g., via a wireless charging pad), by solar energy (e.g., via a solar collector if incorporated in the watch 502), and/or by kinetic motion (e.g., via an internal mechanism if incorporated that can convert kinetic energy into electrical power). In some cases, the battery can be removed, or the battery can be integrated within and/or be a permanent part of the watch 502. In some implementations, the watch 502 includes a connector port for connecting to a charging cable. Such a connector port can additionally be used to connect to cable 508 in some implementations.

The watch 502 (including the strap(s) 501) can be similar or identical to and/or incorporate any of the features described with respect to any of the devices, assemblies, and/or systems described and/or illustrated in U.S. Pat. Pub. No. US2021/0290120 incorporated by reference herein. For example, the watch 502 can include a display 520 similar or identical to the display of the wearable sensor described in U.S. Pat. Pub. No. US2021/0290120 incorporated by reference herein, which can be configured to display a plurality of physiological parameters monitored and/or determined by the pulse oximetry system 500 and/or other information (e.g., such as the time, date, etc.). Further, the display 520 can be configured to receive input from the subject, such as touch input, for interacting with the pulse oximetry system 500. The watch 502 can also include one or more user interfaces 574 similar or identical to the user interfaces 13 (e.g., user interface 13a and/or user interface 13b) of the wearable sensor described in U.S. Pat. Pub. No. US2021/0290120 incorporated by reference herein, which can be configured as button(s). As shown in at least FIG. 14A, such user interfaces 574 can be disposed along a side of the watch 502. As another example and with reference to FIGS. 14C-14D, in some implementations, watch 502 includes a physiological parameter measurement sensor or module 576 that can be similar or identical to any of the physiological parameter measurement sensors or modules described in U.S. Pat. Pub. No. US2021/0290120 incorporated by reference herein. In such implementations, the watch 502 can instruct one or more emitters 512 of the physiological parameter measurement sensor or module 576 to emit light of one or more wavelengths, for example, in tissue of a wrist to which the watch 502 is secured during use. Furthermore, the watch 502 can receive and/or process one or more signals outputted by one or more detectors 514 of the physiological parameter measurement sensor or module 576 and determine one or more physiological parameters based on the received or processed signals.

In implementations of watch 502 that include the physiological parameter measurement sensor or module 576 as shown in FIGS. 14C-14D, such module 576 can work in combination with the one or more emitters and one or more detectors operably positioned by ring member 510. For example, signals and/or data outputted by the module 576 can augment any or all signals and/or data outputted by the one or more detectors operably positioned by ring member 510, such as if signals and/or data outputted by the one or more detectors is interrupted or lost. As another example, the signals and/or data outputted by module 576 can validate any or all signals and/or data outputted by the one or more detectors operably positioned by ring member 510. In a further example, the module 576 can be used as a primary source of signals and/or data for determining one or more physiological parameters of a subject if the cable 508 (and thus the one or more emitters and one or more detectors operably positioned by the ring member 510) is detached from the watch 502. In some implementations, the module 576 can measure physiological parameters of interest separate of and/or distinct from the one or more emitters and one or more detectors operably positioned by ring member 510. In some implementations, the module 576 can be automatically turned off and/or not used when the cable 508 (and thus the one or more emitters and one or more detectors operably positioned by the ring member 510) is connected to the watch 502 (e.g., such as connected at connector port 570). For example, during the day and/or when out in public, a subject can wear the pulse oximetry system 500 without the cable 508 connected. During such use, the module 576 can be used to measure physiological parameters of the subject. Such use can advantageously allow the subject to wear and use the pulse oximetry system in a concealed manner. During the night (e.g., when sleeping) and/or when not in public, a subject can wear the pulse oximetry system 500 with the cable 508 connected. During such use, the one or more emitters and one or more detectors operably positioned by the ring member 510 can be used to measure physiological parameters of the subject. Furthermore, during such use, the module 576 can be automatically turned off, not used, used to measure different physiological parameters than the emitters/detectors operably positioned by the ring member 510, or used to augment measurements of the emitters/detectors operably positioned by the ring member 510. For example, a processor of the watch 502 can determine whether cable 508 is connected to the watch 502 (e.g., via port 570) and disable operation of module 576 when cable 508 is connected, and enable/instruct operation of module 576 when cable 508 is not connected. Further to this example, a processor of the watch 502 can determine whether cable 508 is connected to the watch 502 and modify operation of module 576 when cable 508 is connected, such as to measure different physiological parameters than the emitters/detectors operably positioned by the ring member 510, or to augment measurements of the emitters/detectors operably positioned by the ring member 510.

In some implementations and as shown in FIGS. 14A-14D, the watch 502 includes an ECG sensor that can be utilized (for example, by a processor of the watch 502) to measure an ECG of the subject. The ECG sensor can include a reference electrode, a negative electrode, and a positive electrode, each of which can be electrically isolated from one another and in electrical communication with a processor of the watch 502. As shown in FIGS. 14C-14D, the module 576 can include ECG electrodes 590, one of which can be the reference electrode, the other one of which can be the negative electrode or the positive electrode. As shown in FIGS. 14A-14B, a portion of the watch 502 separated from the module 576 (for example, a portion of an upper surface of the watch 502) can include ECG electrode 578, which can be the positive electrode (if the module 576 includes the negative electrode) or the negative electrode (if the module 576 includes the positive electrode). The ECG electrodes 590 can be located on the module 576 and be configured to contact the subject's skin when the watch 502 is secured around the subject's wrist. Each of the ECG electrodes 590 can be substantially semi-annular/semi-circular and extend along a circumference of the module 576 such that they partially surround the one or more emitters 512 and the one or more detectors 514 of the module 576. When the subject wants to make a measurement using the ECG sensor that includes the ECG electrodes 578, 590, the subject can press on or touch the ECG electrode 578 using a finger or another part of their body such that the subject's skin makes contact with the ECG electrode 578. A processor of the watch 502 can determine when the subject touches the ECG electrode 578 and utilize the ECG electrode 578 and ECG electrodes 590 to measure (for example, automatically measure) the ECG of the subject. In some implementations, the watch 502 further includes an ECG amplifier configured to receive analog signals from the ECG electrodes 578, 590, which can output amplified analog signals to an analog-digital converter that can also be included in the watch 502. The amplified analog signals can include an ECG differential between the positive and negative electrodes. The analog-digital converter can output a digital signal based on the analog signals from the ECG electrodes 578, 590 to the processor of the watch 502 for determination of the subject's ECG. In some implementations, the ECG sensor can optionally make physiological measurements based on the obtained ECG, for example, a heart rate, a respiratory rate, and/or otherwise of the subject. The subject's ECG waveform and/or the other physiological measurements made from the ECG sensor can be displayed on the display 520 and/or transmitted to a separate device in communication with the watch 502 for display.

FIGS. 14E-14F illustrate an implementation of securement features of the straps 501. As discussed herein and as shown, a buckle 572 (for example, having a buckle tongue) can be coupled to a first one of the straps 501 (labeled 501a in FIGS. 14C-14F). A strap loop 573 can be coupled to such strap 501a as shown. The buckle 572 and the strap loop 573 can receive a free end of a second one of the straps 501 (labeled 501b in FIGS. 14C-14D) that is secured by the buckle 572. As shown in at least FIGS. 14C-14D, the straps 501a, 501b can have one or more strap holes 577 disposed along at least a portion of their length. As further shown in FIGS. 14E-14F, the strap loop 573 can have one or more strap loop protrusions 575 configured to secure to one or more strap holes 577 in strap 501b, for example, when strap 501b is received by the strap loop 573. The strap loop 573 can thus be used to secure the free end of the strap 501b. In an alternative implementation (not shown), the strap 501b that is secured by the buckle 572 can have one or more protrusions (for example, adjacent its free end) configured to interact with one or more holes of strap loop 573 to secure the free end of the strap 501b. In some implementations, the strap loop 573 secures the free end of the strap 501b that is secured by the buckle 572 without protrusions or holes. Any of the implementations of a pulse oximetry system described herein can include a strap the same or similar to the strap 501 and/or include any of the features of the strap 501, such as one or more strap holes 577 and/or a strap loop 573.

FIGS. 14G-14H illustrate a pulse oximetry system 500′ that is a variant of the pulse oximetry system 500 described and illustrated with respect to FIGS. 14A-14D. FIGS. 14G-14H illustrate bottom perspective views of the pulse oximetry system 500′. The pulse oximetry system 500′ is identical to the pulse oximetry system 500 and includes all features and/or functionality of the pulse oximetry system 500 except that it does not include the physiological parameter measurement sensor or module 576. For example, the pulse oximetry system 500′ can include straps 501′, buckle 572′, strap loop 573′, cable 508′ with first portion 507′ and second portion 509′, ring member 510′, wrist portion 502′, connector port 570′, and user interface(s) 574′ which can be similar or identical to straps 501, buckle 572, strap loop 573, cable 508 with first portion 507 and second portion 509, ring member 510, wrist portion 502, connector port 570, and user interface(s) 574 (respectively) described above with respect to the pulse oximetry system 500. In some implementations, the cable 508′ (and the one or more emitters and one or more detectors operably positioned by the ring member 510′) is permanently connected to the watch 502′.

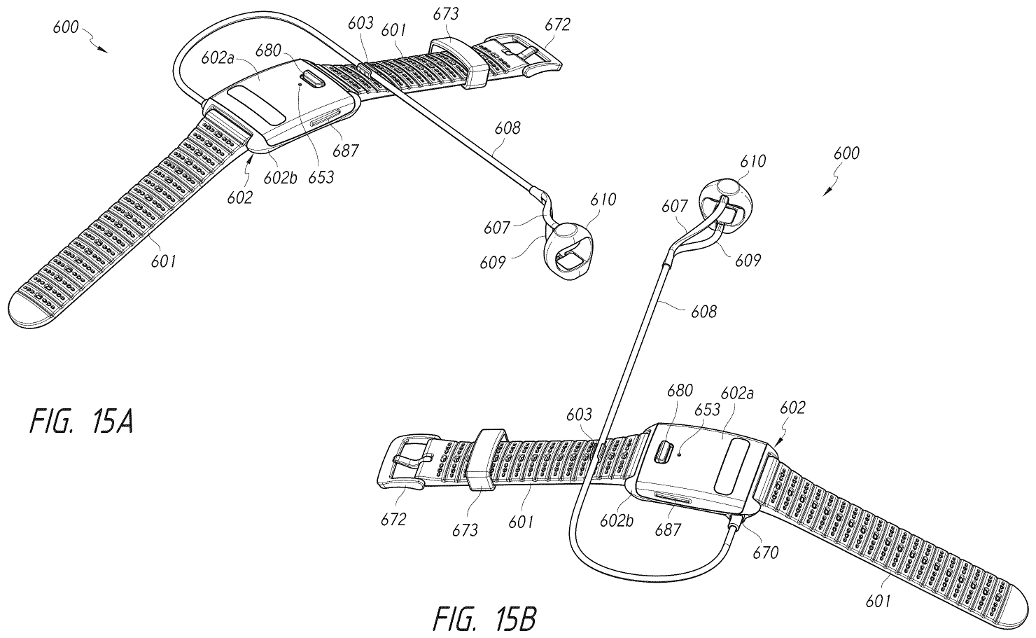

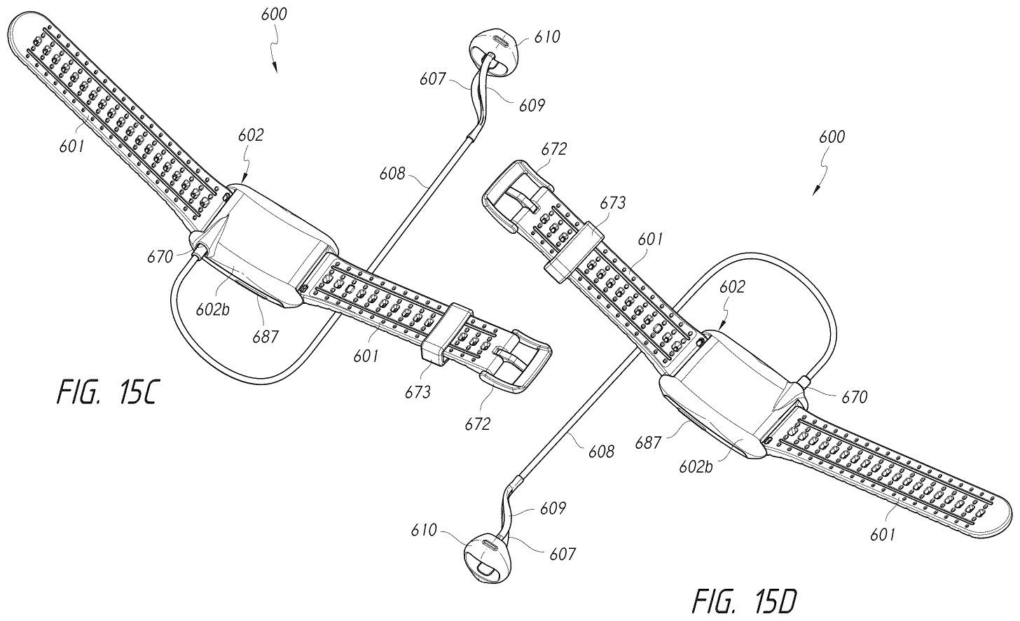

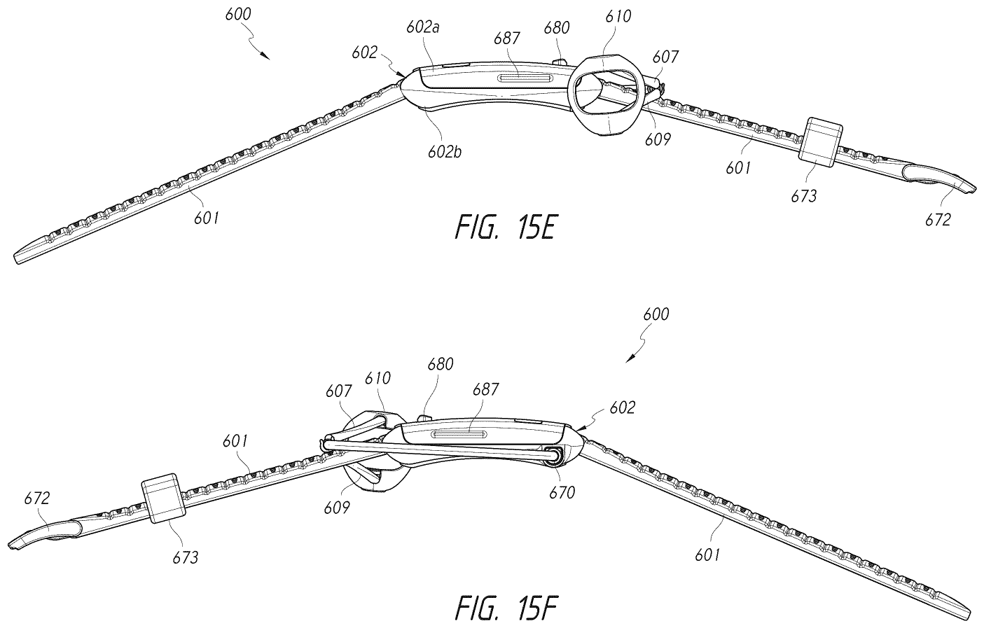

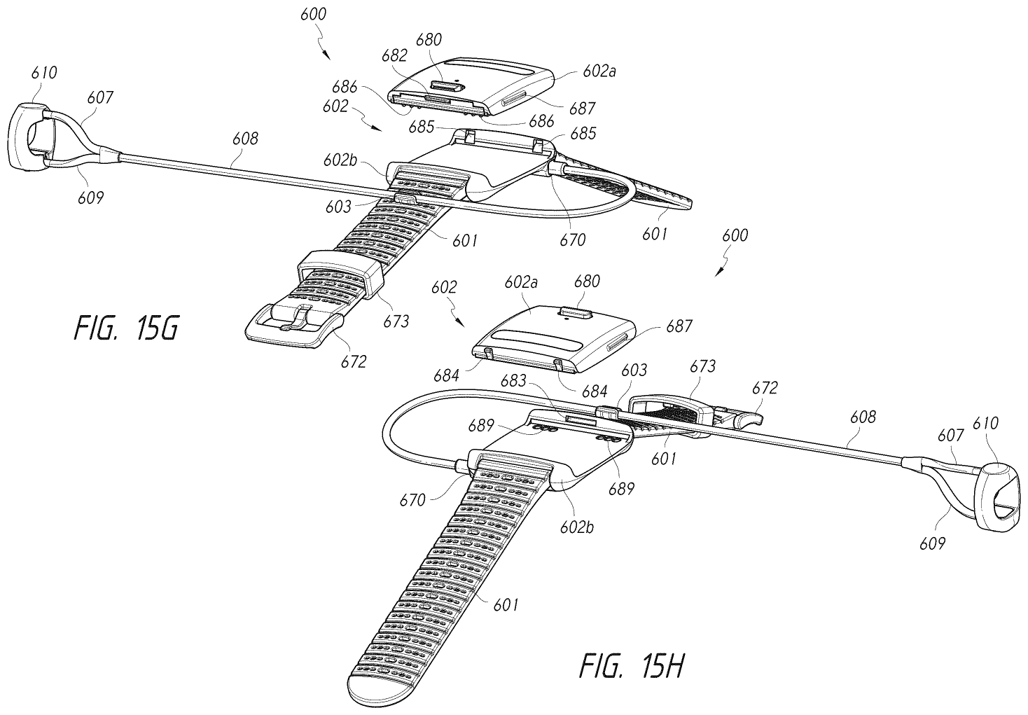

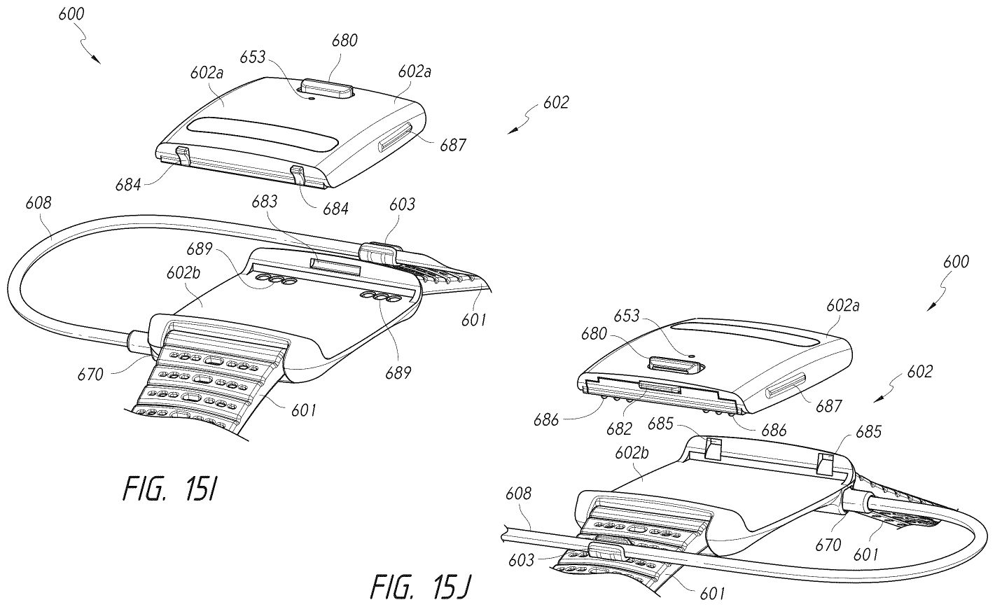

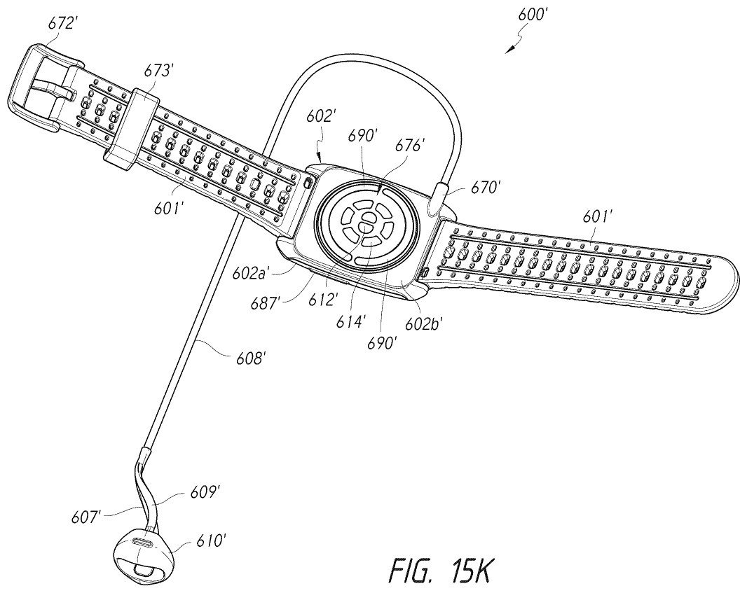

FIGS. 15A-15B illustrate top perspective views, FIGS. 15C-15D illustrate bottom perspective views, and FIGS. 15E-15F illustrate side views of another implementation of a pulse oximetry system 600. Pulse oximetry system 600 includes a wrist portion 602, straps 601 coupled to wrist portion 602, a cable 608, and a ring member 610. Straps 601 can be similar or identical to straps 501 discussed above. Cable 608 can be similar or identical to any of cables 108, 408, and/or 508 discussed above. Cable 608 can split into first and second portions 607, 609 similar or identical to cables 108, 408, and 508 discussed elsewhere herein. Similar to that discussed with respect to cables 108, 408, and 508, cable 608 can operably connect wrist portion 602 with an emitter housing and/or emitter package comprising one or more emitters (similar and/or identical to the those described herein, such as emitter package 134 comprising one or more emitters 112) and a detector housing and/or a detector package comprising one or more detectors (similar and/or identical to the those described herein, such as detector package 136 comprising one or more detectors 114). Ring member 610, which can operably position such emitter housing and/or emitter package and detector housing and/or detector package, can be similar or identical to any of the ring members discussed elsewhere herein, such as ring member 110, 410, and/or 510. In some implementations, the wrist portion 602 can include one or more status indicators similar or identical to status indicator 102g described herein, which can include status indicator 167 described herein. The one or more status indicators 102g/167 can be configured, for example, to emit optical radiation out of and/or through a hole and/or opening in the wrist portion 602, such as through the hole/opening 653 in a top of the wrist portion 602.

In some implementations and as shown in FIGS. 15G-15H, wrist portion 602 comprises a first component 602a (which may also be referred to as a “sensor hub” or “hub”) and a second component 602b (which may also be referred to as a “dock”). Hub 602a can be configured to removably secure to dock 602b, and vice versa, for example, via a latch mechanism 680. Movement of the latch mechanism 680, for example, by a user's finger, can disconnect hub 602a and dock 602b from one another. In some implementations and as shown in FIGS. 15G-15H and magnified views thereof in FIGS. 15I-15J, the latch mechanism 680 includes a protrusion 682 that, in a resting state of the latch mechanism 680, extends outward from the hub 602a (for example, through an opening of hub 602a) that can engage with a cavity 683 of the dock 602b to connect the hub 602a and the dock 602b to one another. In some implementations, latch mechanism 680 is biased toward such resting state, which may be referred to as an extended position. When the latch mechanism 680 is moved, for example, inward towards the center of the hub 602a (which may be referred to as a retracted position of latch mechanism 680), the protrusion 682 can correspondingly move inward towards the center of the hub 602a and effectively pull away from the cavity 683 to release the hub 602a and the dock 602b from one another. In addition to the latch mechanism 680, the hub 602a can include one or more protrusions 684 at an end opposite of the latch mechanism 680 for engaging with one or more corresponding cavities 685 of the dock 602b. Thus in some implementations, to connect the hub 602a and dock 602b to one another, the one or more protrusions 684 can be inserted into the one or more cavities 685, and the hub 602a and the dock 602b can be brought together (e.g., such as by pushing the hub 602a downward into the dock 602b) causing the protrusion 682 of the latch mechanism 680 to engage with the cavity 683 of the dock 602b. In some implementations, the protrusion 682 has a ramp-like lower surface that inclines in a direction away from the hub 602a as shown that, when the hub 602a and the dock 602b are brought together, causes the protrusion 682 and the latch mechanism 680 to move inwards towards the center of the hub 602a. Upon the hub 602a and the dock 602b coming together fully, the protrusion 682 can extend outward and engage with the cavity 683, securely connecting the hub 602a and the dock 602b to one another. Thus, in some implementations, the hub 602a can snap into engagement with dock 602b. To facilitate such engagement, the latch mechanism 680 can be spring-loaded or otherwise biased to its resting state (e.g., wherein the protrusion 682 extends outward from the hub 602a). In some variants, hub 602a and dock 602b are configured to connect and disconnect to one another without a latch mechanism (for example, without latch mechanism 680). For example, hub 602a and dock 602b can include protrusions that provide for a snap-fit engagement/disengagement, without a movable latch mechanism.

In some implementations and as shown in FIGS. 15G-15J, the hub 602a can include one or more electrical connectors 686 and the dock 602b can include one or more electrical connectors 689 configured to electrically connect with one another when the hub 602a and the dock 602b are connected to one another. As shown, the electrical connector(s) 686 of the hub 602a can be disposed at an underside/bottom of the hub 602a and the electrical connector(s) 689 of the dock 602b can be disposed at a topside/top of the dock 602b. The electrical connector(s) 686 can be configured to have a spring force or bias that pushes ends of the electrical connector(s) 686 away from the hub 602a. Such a spring force/bias can aid in making electrical connections between the electrical connector(s) of the hub 602a with the corresponding electrical connector(s) 689 of the dock 602b. In some implementations, the electrical connector(s) 686 can be configured as pogo-like spring electrical connectors as shown. Also as shown, the electrical connector(s) 689 of the dock 602b can be configured as recesses configured to receive the electrical connector(s) 686 of the hub 602a. The hub 602a can include 6 electrical connectors 686 and the dock 602b can include 6 electrical connectors 689 as shown, although in some implementations the hub and dock can include less than 6 electrical connectors each or more than 6 electrical connectors each. Such electrical connectors when connected (e.g., when the hub 602a and the dock 602b are secured to one another) can facilitate communication between components of the hub 602a and components of the dock 602b and/or any components connected to either the hub 602a and the dock 602b.

Dock 602b can be coupled (for example, permanently coupled) to straps 601. In some implementations, hub 602a can include a rechargeable battery, and hub 602a can be removed from dock 602b, for example, in order to facilitate charging of hub 602a. In some implementations, dock 602b does not include a battery. Hub 602a can include any or all of the components discussed with respect to sensor hub 102 and FIG. 2C, for example, processor(s) 102a, storage device(s) 102b, communication module 102c, battery 102d, vibration motor 102f, information element 102e, and/or other sensor(s) 102h (such as an accelerometer, gyroscope, temperature sensor, among others).

With reference to FIGS. 15A-15J, in some implementations, hub 602a includes a rib 687 extending along a surface of hub 602a on one or both sides of hub 602a that allow a user to grip hub 602a to aid removal from dock 602b. In some implementations, with reference to FIGS. 15E-15F, a bottom of wrist portion 602 (for example, a bottom of dock 602b) is curved, for example, curved between portions of dock 602b that are coupled to ends of straps 601, which can help wrist portion 602 to better conform to a subject's wrist. In some implementations, wrist portion 602 (for example, hub 602a) can receive and/or process one or more signals outputted by one or more detectors operably positioned by ring member 610 and determine one of more physiological parameters based on the received or processed signals. In some implementations, wrist portion 602 (for example, hub 602a) can instruct one or more emitters operably positioned by ring member 610 to emit light of one or more wavelengths, for example, in tissue of a finger to which ring member 610 is secured during use.

In some implementations, cable 608 is detachable from the wrist portion 602, for example, removably connectable to a connector port 670 of wrist portion 602 (see FIG. 15B-15D). Such connector port 670 can be part of dock 602b, or alternatively, hub 602a. In some implementations, cable 608 is permanently attached to wrist portion 602 (for example, permanently fixed to dock 602b or hub 602a). The cable 608 can be secured to and/or positioned along a subject similar or identical to that discussed with respect to cable 108 in pulse oximetry system 100, for example, with the utilization of one or more cable retainers (such as cable retainers 103, 105) and/or similar to that discussed above with respect to pulse oximetry system 100. In some implementations, pulse oximetry system 600 includes a cable retainer 603 that can be similar or identical to any of the cable retainers discussed herein, such as cable retainer 403 and/or 503. Cable retainer 603 can be permanently or removably attached to one of straps 601. A buckle 672 (for example, having a buckle tongue) can be coupled to one of the straps 601 to allow the straps 601 to be secured to one another (for example, around a subject's wrist), although the straps 601 can be secured to one another in other ways (e.g., via magnets, via the straps having a shape memory, or the like). The straps 601 can also include any of the features as described with respect to the straps 501, such as a strap loop 673 the same or similar to the strap loop 573, and strap holes the same or similar to the strap holes 577.

FIG. 15K illustrates a pulse oximetry system 600′ that is a variant of the pulse oximetry system 600 described and illustrated with respect to FIGS. 15A-15J. FIG. 15K illustrates a bottom perspective view of the pulse oximetry system 600′. The pulse oximetry system 600′ can be the same or similar to the pulse oximetry system 600 and can include any or all features and/or functionality of the pulse oximetry system 600. For example, the pulse oximetry system 600′ can include straps 601′, buckle 672′, strap loop 673′, cable 608′ with first portion 607′ and second portion 609′, ring member 610′, wrist portion 602′ with hub 602a′ and dock 602b′, connector port 670′, and rib(s) 687′ which can be similar or identical to straps 601, buckle 672, strap loop 673, cable 608 with first portion 607 and second portion 609, ring member 610, wrist portion 602 with hub 602a and dock 602b, connector port 670, and rib(s) 687 (respectively) described above with respect to the pulse oximetry system 600. The pulse oximetry system 600′ differs from the pulse oximetry system 600 in that it includes a physiological parameter measurement sensor or module 676′ (for example, operably positioned at a bottom of a dock 602b′ of system 600′) and an ECG sensor that can be similar or identical to any of the physiological parameter measurement sensors or modules and ECG sensors described in U.S. Pat. Pub. No. US2021/0290120 incorporated by reference herein and/or with respect to pulse oximetry system 500. For example, the module 676′ can include one or more emitters 612′, one or more detectors 614′, and/or ECG electrodes 690′ that are the same or similar to and can include any or all features and/or functionality of the one or more emitters 512, the one or more detectors 514, and/or the ECG electrodes 590 of module 576. Furthermore, the pulse oximetry system 600′ can include an ECG electrode 678 (not shown) that can be similar or identical to the ECG electrode 578 described above with respect to pulse oximetry system 500 or to the ECG electrode 678″ described below with respect to pulse oximetry system 600″. Pulse oximetry system 600′ can be referred to as a “wearable device” and can include, for example, a watch configured to wrap around a subject's wrist. In some implementations, module 676′ is part of dock 602b′ of wrist portion 602′, or alternatively, is part of hub 602a′ that removably secures to dock 602b′.

The module 676′ can work in combination with the one or more emitters and one or more detectors operably positioned by ring member 610′. For example, signals and/or data outputted by the module 676′ can augment any or all signals and/or data outputted by the one or more detectors operably positioned by ring member 610′, such as if signals and/or data outputted by the one or more detectors is interrupted or lost. As another example, the signals and/or data outputted by module 676′ can validate any or all signals and/or data outputted by the one or more detectors operably positioned by ring member 610. In a further example, the module 676′ can be used as a primary source of signals and/or data for determining one or more physiological parameters of a subject if the cable 608′ (and thus the one or more emitters and one or more detectors operably positioned by the ring member 610′) is detached from the pulse oximetry system 600′. In some implementations, the module 676′ can measure physiological parameters of interest separate of and/or distinct from the one or more emitters and one or more detectors operably positioned by ring member 610′. In some implementations, the module 676′ can be automatically turned off and/or not used when the cable 608′ (and thus the one or more emitters and one or more detectors operably positioned by the ring member 610′) is connected to wrist portion 602′ of the pulse oximetry system 600′ (e.g., such as connected at connector port 670′). For example, during the day and/or when out in public, a subject can wear the pulse oximetry system 600′ without the cable 608′ connected. During such use, the module 676′ can be used to measure physiological parameters of the subject. Such use can advantageously allow the subject to wear and use the pulse oximetry system in a concealed manner. During the night (e.g., when sleeping) and/or when not in public, a subject can wear the pulse oximetry system 600′ with the cable 608′ connected. During such use, the one or more emitters and one or more detectors operably positioned by the ring member 610′ can be used to measure physiological parameters of the subject. Furthermore, during such use, the module 676′ can be automatically turned off, not used, used to measure different physiological parameters than the emitters/detectors operably positioned by the ring member 610′, or used to augment measurements of the emitters/detectors operably positioned by the ring member 610′. For example, a processor of the wrist portion 602′ can determine whether cable 608′ is connected to the wrist portion 602′ (e.g., via port 670′) and disable operation of module 676′ when cable 608′ is connected, and enable/instruct operation of module 676′ when cable 608′ is not connected. Further to this example, a processor of the wrist portion 602′ can determine whether cable 608′ is connected to the wrist portion 602′ and modify operation of module 676′ when cable 608′ is connected, such as to measure different physiological parameters than the emitters/detectors operably positioned by the ring member 610′, or to augment measurements of the emitters/detectors operably positioned by the ring member 610′.

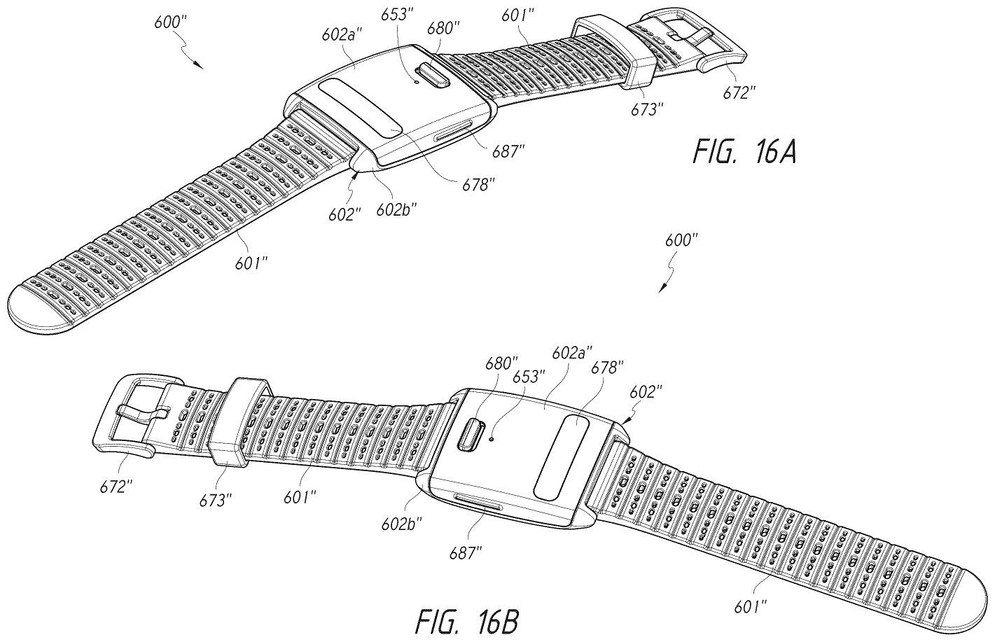

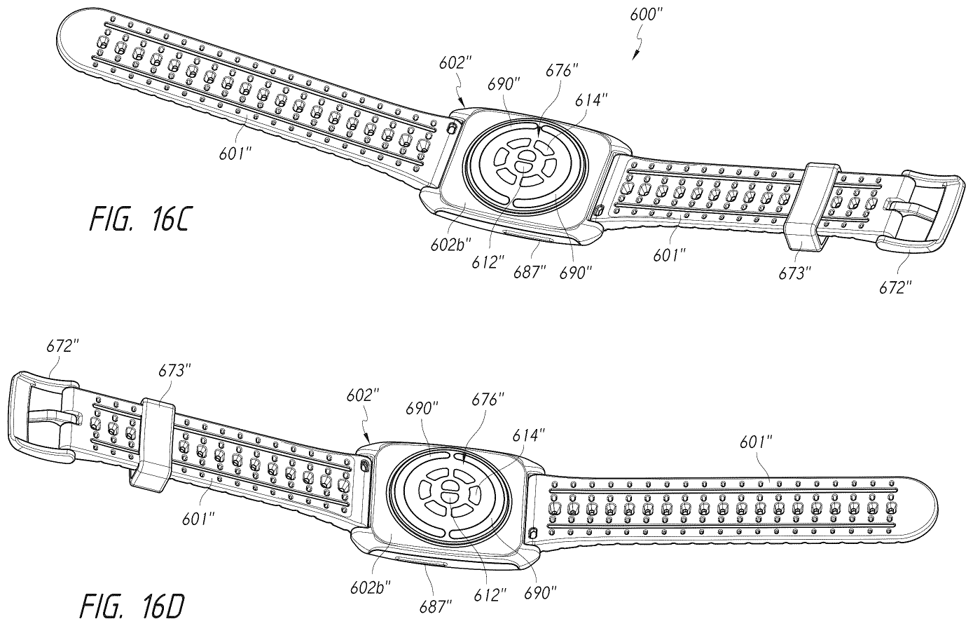

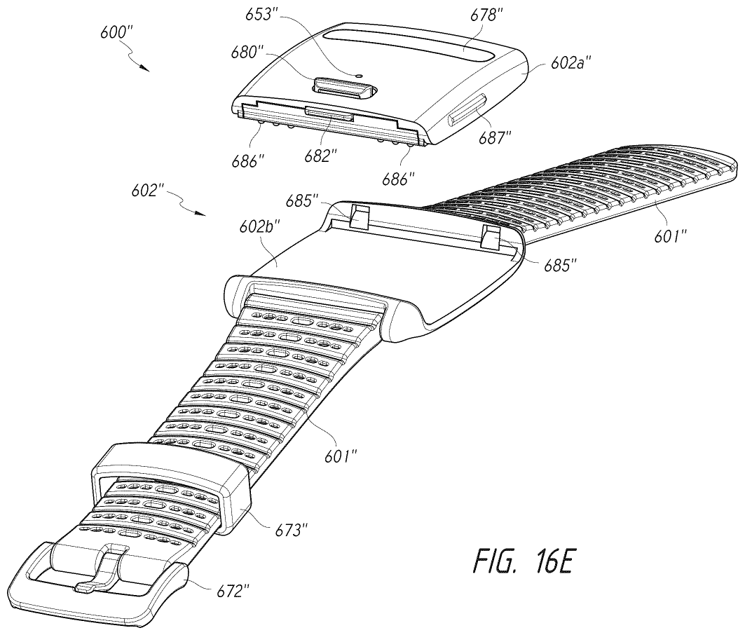

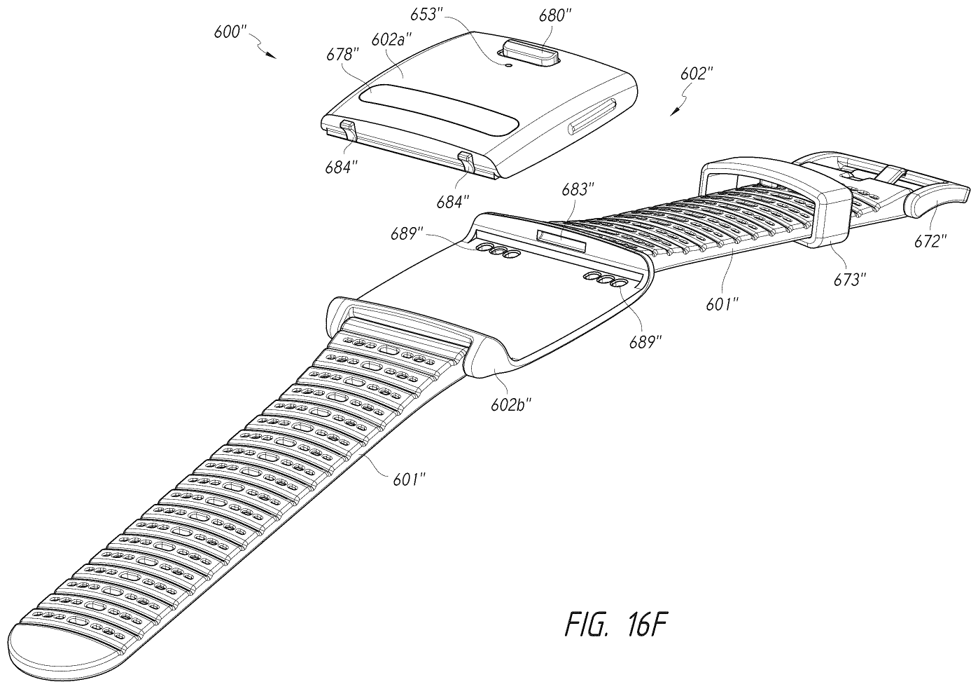

FIGS. 16A-16F illustrate a pulse oximetry system 600″ that is a variant of the pulse oximetry system 600 described and illustrated with respect to FIGS. 15A-15J. FIGS. 16A-16B illustrate top perspective views of the pulse oximetry system 600″, FIGS. 16C-16D illustrate bottom perspective views of the pulse oximetry system 600″, and FIGS. 16E-16F show top perspective views of the pulse oximetry system 600″ with a hub portion 602a″ separated from a dock portion 602b″. The pulse oximetry system 600″ can be the same or similar to the pulse oximetry system 600 and can include any or all features and/or functionality of the pulse oximetry system 600. For example, the pulse oximetry system 600″ can include straps 601″, buckle 672″, strap loop 673″, wrist portion 602″ with hub 602a″ and dock 602b″, rib(s) 687″, hole/opening 653″, and latch mechanism 680″ which can be similar or identical to straps 601, buckle 672, strap loop 673, wrist portion 602 with hub 602a and dock 602b, rib(s) 687, hole/opening 653, and latch mechanism 680 (respectively) described above with respect to the pulse oximetry system 600. The pulse oximetry system 600″ differs from the pulse oximetry system 600 in that, instead of having the port 670, the cable 608 with first and second portions 607 and 609, and the ring member 610, it can include a physiological parameter measurement sensor or module 676″ (for example, operably positioned at a bottom of dock 602b″ of system 600″) and an ECG sensor that can be similar or identical to any of the physiological parameter measurement sensors or modules and ECG sensors described in U.S. Pat. Pub. No. US2021/0290120 incorporated by reference herein and/or with respect to pulse oximetry systems 500 and/or 600′. For example, the module 676″ can include one or more emitters 612″, one or more detectors 614″, and/or ECG electrodes 690″ that are the same or similar to and can include any or all features and/or functionality of the one or more emitters 512, 612′, the one or more detectors 514, 614′, and/or the ECG electrodes 590, 690′, of modules 576, 676′. Furthermore, the pulse oximetry system 600″ can include an ECG electrode 678″ that can be similar or identical to the ECG electrode 578 described above with respect to pulse oximetry system 500. As shown in at least FIGS. 16A-16B, a portion of the wrist portion 602″ separated from the module 676″ (for example, a portion of an upper surface of the hub 602a″) can include the ECG electrode 678″. Pulse oximetry system 600″ can be referred to as a “wearable device” and can include, for example, a watch configured to wrap around a subject's wrist. In some implementations, module 676″ is part of dock 602b″ of wrist portion 602″, or alternatively, is part of hub 602a″ that removably secures to dock 602b″.

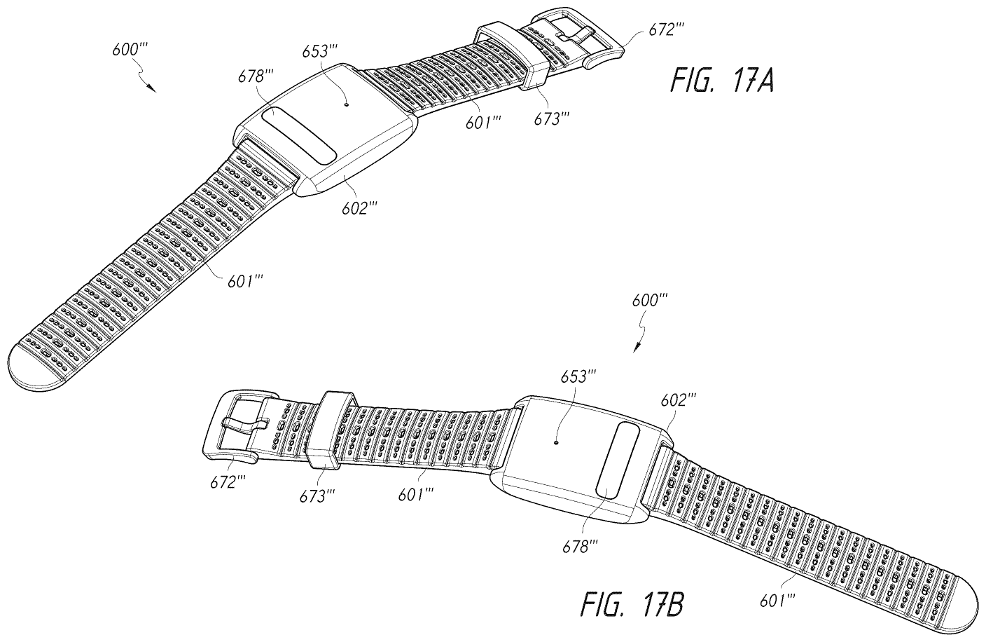

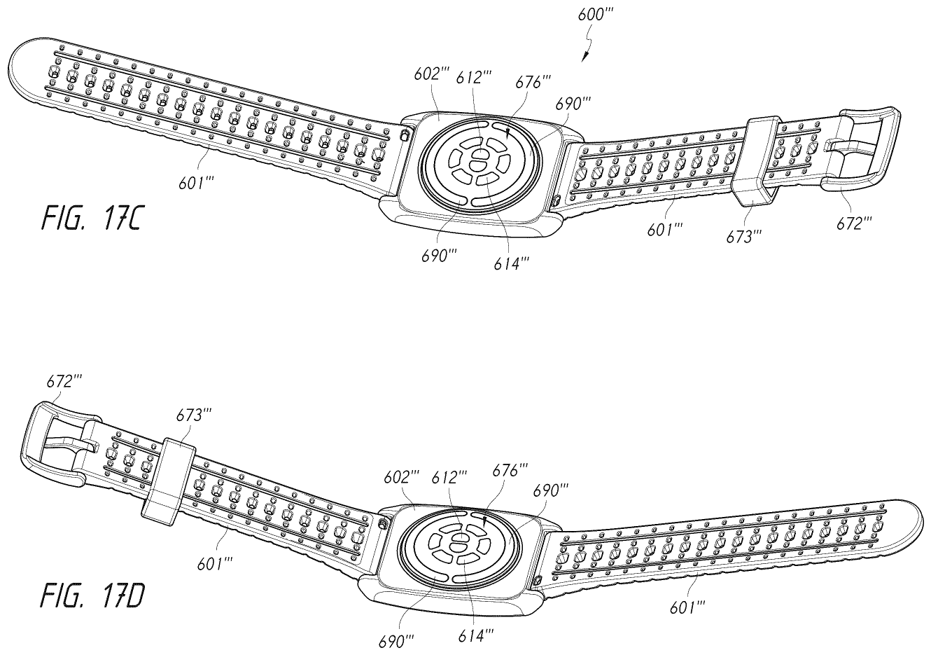

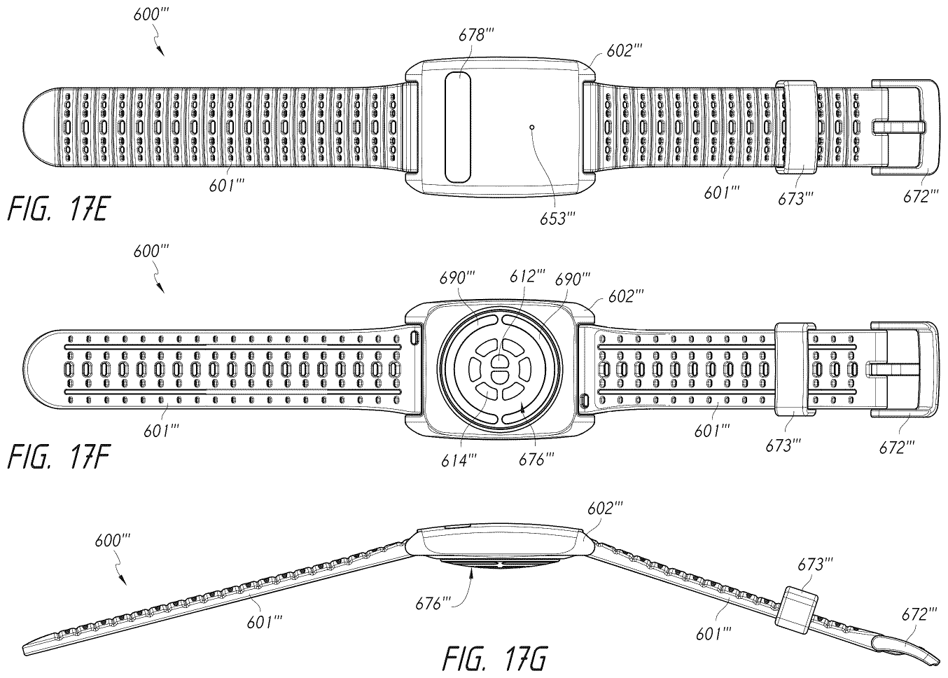

FIGS. 17A-17G illustrate a pulse oximetry system 600″′ that is a variant of the pulse oximetry systems 600′ and 600″ described with respect to FIG. 15K and FIGS. 16A-16F, respectively. FIGS. 17A-17B illustrate top perspective views, FIGS. 17C-17D illustrate bottom perspective views, and FIGS. 17E-17G illustrate a top, bottom, and side view, respectively, of the pulse oximetry system 600″′. The pulse oximetry system 600″′ can be the same or similar to the pulse oximetry systems 600′ and/or 600″ and can include any or all features and/or functionality of the pulse oximetry systems 600′ and/or 600″. For example, the pulse oximetry system 600″′ can include straps 601″′, buckle 672″′, strap loop 673″′, and hole/opening 653″′ which can be similar or identical to straps 601″, buckle 672″, strap loop 673″, and hole/opening 653″ (respectively) described above with respect to the pulse oximetry system 600″. Furthermore, the pulse oximetry system 600″′ can include a physiological parameter measurement sensor or module 676″′ and ECG sensor that can be similar or identical to the physiological parameter measurement sensor or module 676′ and/or 676″ and ECG sensor of pulse oximetry systems 600′ and/or 600″ and/or any of the physiological parameter measurement sensors or modules and ECG sensors described in U.S. Pat. Pub. No. US2021/0290120 incorporated by reference herein. For example, the pulse oximetry system 600″′ can include an ECG sensor including ECG electrodes 690″′, 678″′ that can be similar or identical to the ECG sensor including ECG electrodes 690″, 678″ described with respect to the pulse oximetry system 600″. The pulse oximetry system 600″′ differs from the pulse oximetry systems 600′ and 600″ in that wrist portion 602″′ has a unitary structure instead of separate/separable components, in contrast to wrist portion 602′/602″ that can include a separable hub 602a′/602a″ and a dock 602b′/602b″. However, wrist portion 602″′ can include any or all features and/or functionality described with respect to any of the hubs and/or docks described elsewhere herein (for example, with respect to hub 602a′/602a″ and dock 602b′/602b″). In some variations (not shown), the pulse oximetry system 600″′ can include a cable, a ring member, and emitters/detectors operably positioned by the ring member similar or identical to the cable 608, ring member 610, and emitters/detectors operably positioned by the ring member 610 described and illustrated with respect to FIGS. 15A-15J and/or FIG. 15K.

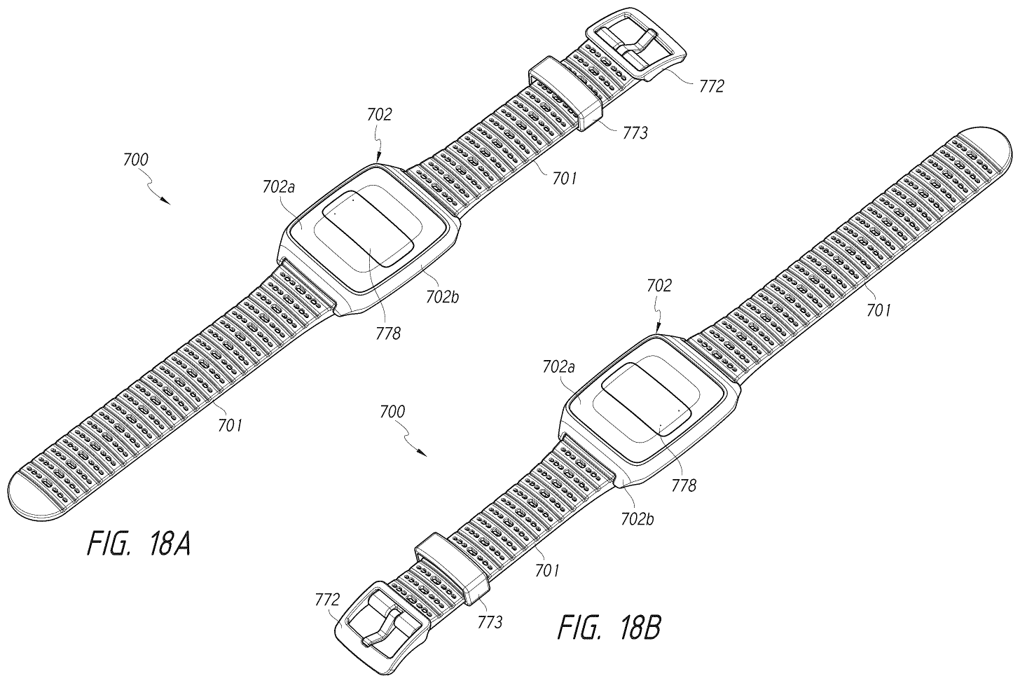

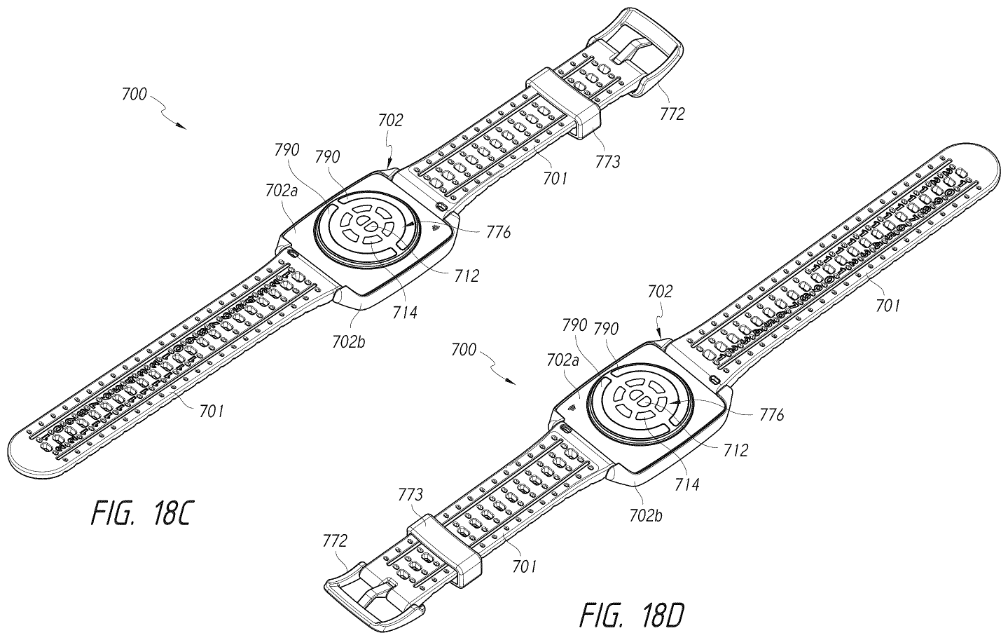

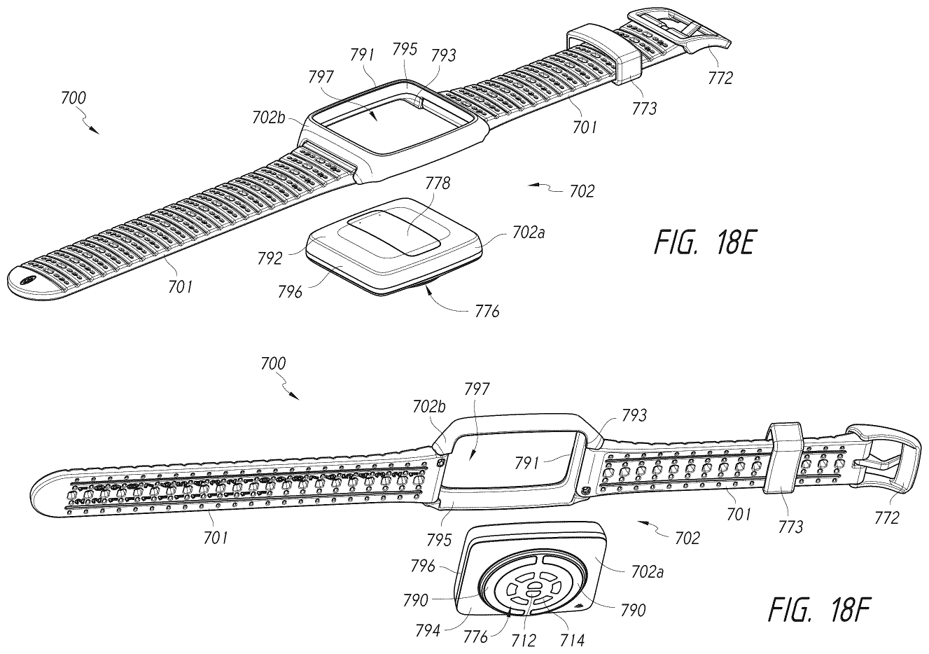

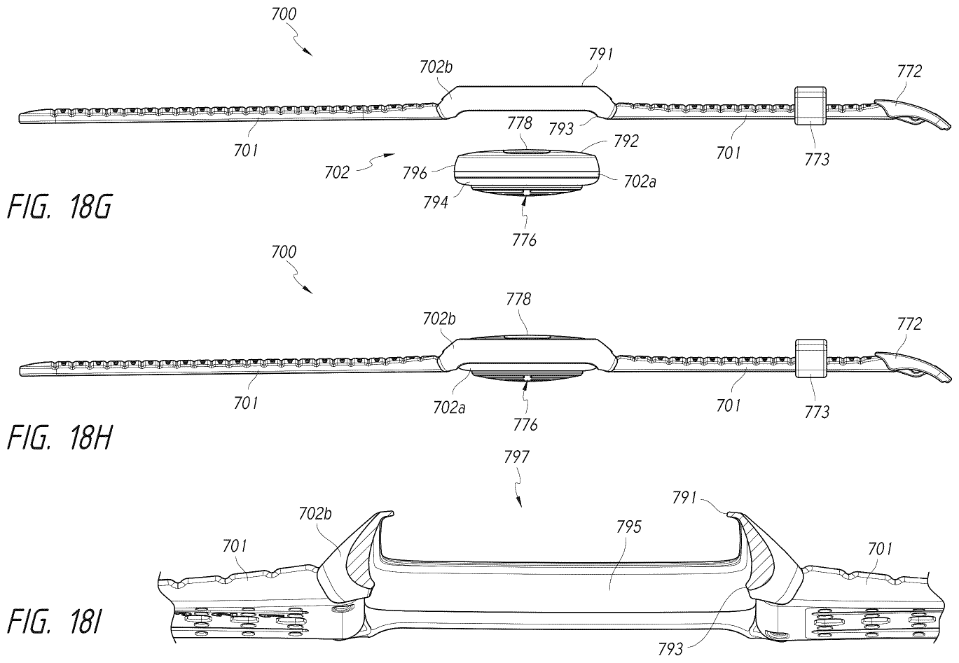

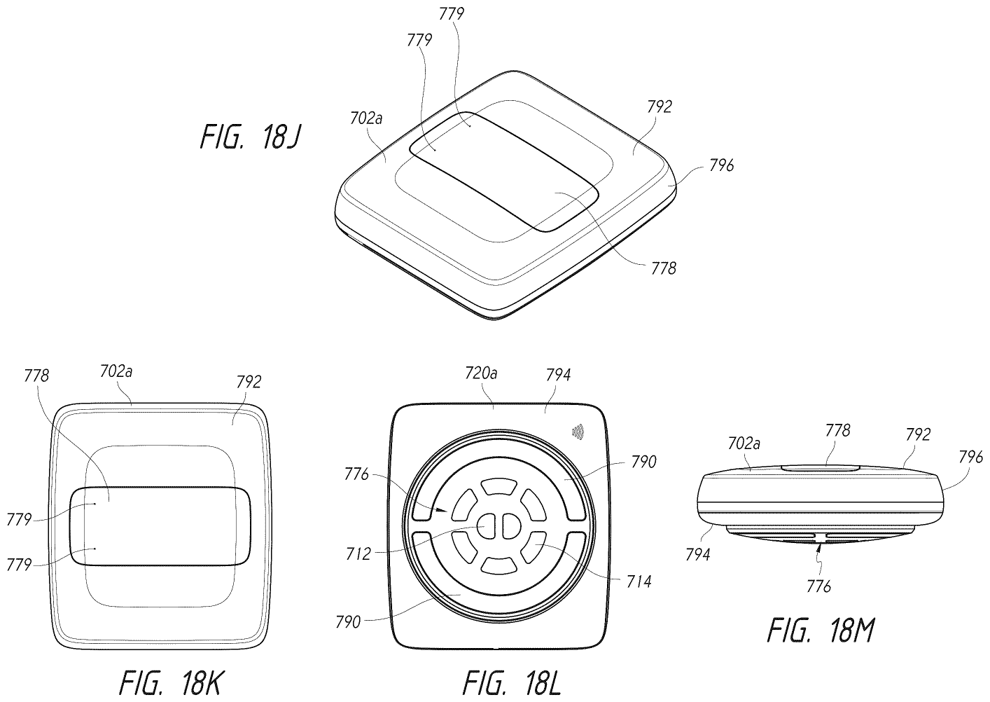

FIGS. 18A-18M illustrate various views of another implementation of a pulse oximetry system 700 configured to be secured to a subject's wrist 3. FIGS. 18A-18B illustrate top perspective views and FIGS. 18C-18D illustrate bottom perspective views of the pulse oximetry system 700. The pulse oximetry system 700 can have similar and/or the same features, aspects, and/or components as any of the pulse oximetry systems described herein, such as pulse oximetry systems 100, 400, 500, 600 and variants thereof. For example, the pulse oximetry system 700 can have a wrist portion 702 configured similar to the sensor dock 106 and the sensor hub 102 (e.g., the wrist portion 702 can combine any and/or all aspects of the sensor dock 106 and the sensor hub 102). The wrist portion 702 can be configured to be secured to the wrist 3 of the subject 1 similar and/or the same as how the sensor dock 106 can be secured to the wrist 3 of the subject 1, for example, by a strap 701 similar and/or the same as the strap 101 and/or any of the straps 401, 501, 601 described herein. Furthermore, the pulse oximetry system 700 can be similar or identical to and/or incorporate any of the features described and/or illustrated in U.S. Pat. Pub. No. US2021/0290120 incorporated by reference herein. For example, the pulse oximetry system 700 can include a physiological parameter measurement sensor or module 776 that can be similar or identical to any of the physiological parameter measurement sensors or modules described in U.S. Pat. Pub. No. US2021/0290120 incorporated by reference herein and/or with respect to modules 576, 676′, 676″, and/or 676″′. Furthermore, the pulse oximetry system 700 can include an ECG sensor that can be similar or identical to any of the ECG sensors of the pulse oximetry systems described herein, such as pulse oximetry systems 500, 600′, 600″, and/or 600″′. For example, the module 776 can include one or more emitters 712, one or more detectors 714, and ECG electrodes 790 that are the same or similar to and can include any or all features and/or functionality of the one or more emitters 512, 612″, 612″, 612″′ the one or more detectors 514, 614′, 614″, 614″′, and/or the ECG electrodes 590, 690′, 690″, 690″′, of modules 576, 676′, 676″, 676″′. Furthermore, the pulse oximetry system 700 can include an ECG electrode 778 that can be similar or identical to the ECG electrode 678″ described above with respect to pulse oximetry system 600″. As shown in at least FIGS. 18A-18B, a portion of the wrist portion 702 separated from the module 776 (for example, a portion of an upper surface of the hub 702a) can include the ECG electrode 778. The wrist portion 702 can instruct the one or more emitters 712 of the physiological parameter measurement sensor or module 776 to emit light of one or more wavelengths, for example, in tissue of a wrist to which the wrist portion 702 is secured during use. Furthermore, the wrist portion 702 can receive and/or process one or more signals outputted by the one or more detectors 714 of the physiological parameter measurement sensor or module 776 and determine one or more physiological parameters based on the received or processed signals.

In some implementations and as shown in the perspective views of FIGS. 18E-18F and side views of FIGS. 18G-18H, wrist portion 702 comprises a first component 702a (which may also be referred to as a “sensor hub” or “hub”) and a second component 702b (which may also be referred to as a “dock”). Hub 702a can be configured to removably secure to dock 702b, and vice versa, such as by a snap fit. For example, the hub 702a can secure to the dock 702b via a snap fit by being pushed into the dock 702b from below, and be removed from the dock 702b by being pushed down from above relative to the dock 702b. For this releasably connection, the dock 702b can have an opening 797 configured to removably receive the hub 702a, the opening 797 defined by an upper ridge 791, a lower ridge 793, and an inner portion 795 spanning between the upper ridge 791 and the lower ridge 793 as shown in FIG. 18I. The upper ridge 791 can be a continuous ridge along a top portion of the dock 702b that prevents/limits further upward movement of the hub 702a within the dock 702b when the hub 702a and dock 702b are secured to one another. For example, a portion of a top 792 of the hub 702a can seat against the upper ridge 791 of the dock 702b when the hub 702a and dock 702b are secured to one another. The inner portion 795 of the dock 702b can be configured to receive a side 796 of the hub 702a when the hub 702a and dock 702b are secured to one another. As such, the inner portion 795 of the dock 702b can mirror the side 796 of the hub 702a. The lower ridge 793 of the dock 702b can be configured as a discontinuous ridge along a bottom portion of the dock 702b that allows the hub 702a to be removably received by the dock 702b and secures the hub 702a within the dock 702b when the hub 702a and dock 702b are secured to one another. For example, a portion of a bottom 794 of the hub 702a can seat against the lower ridge 793 of the dock 702b when the hub 702a and dock 702b are secured to one another. For the releasable snap fit between the hub 702a and the dock 702b, the dock 702b can be formed of a resilient material that allows some flexion/deformation of the dock 702b when securing or removing the hub 702a and the dock 702b to/from one another.

Additionally, and as shown in the top perspective, top, and bottom views of the hub 702a in FIGS. 18J-18L, respectively, when viewed from above the hub 702a can have a generally rounded rectangular shape. Such a rounded rectangular shape can allow the hub 702a to fit into the dock 702b in two orientations (each 180 degrees from each other), which can advantageously allow for the hub 702a to be positioned in the dock 702b as desired on either a left wrist or a right wrist of a subject. When viewed from the side, such as shown in FIG. 18M, the hub 702a can have a rounded side 796 with rounded transitions between the side 796 and the top 792 and the side 796 and the bottom 794. Additionally, the side 796 of the hub 702a can taper inwards towards the center of the hub 702a from near the bottom 794 to the top 792, which can facilitate securement and removal of the hub 702a with the dock 702b. In some implementations and as shown in at least FIG. 18J-18K, the hub 702a can include one or more holes 779 configured to allow optical radiation emitted by one or more status indicators of the hub 702a to be visible from a location external to the hub 702a, such as by the subject 1 when wearing/using the pulse oximetry system 700. In some implementations, holes/openings 779 can be at least partially aligned with such status indicator(s) to allow optical radiation emitted from the status indicator(s) to more easily pass through the holes/openings 779. Such status indicator(s) can be LEDs, for example.

The dock 702b can be coupled (for example, permanently coupled) to straps 701. In some implementations, hub 702a can include a rechargeable battery, and hub 702a can be removed from dock 702b, for example, in order to facilitate charging of hub 702a. In some implementations, dock 702b does not include a battery, hardware processor(s), and/or sensors. Hub 702a can include any or all of the components discussed with respect to sensor hub 102 and FIG. 2C, for example, processor(s) 102a, storage device(s) 102b, communication module 102c, battery 102d, vibration motor 102f, information element 102e, and/or other sensor(s) 102h. In some implementations, dock 702b does not include any electronic components (for example, does not include any electrical circuitry).

The pulse oximetry system 700 can be powered by a battery. In some implementations, the hub 702a can be powered by a battery. The battery can be rechargeable. The battery can be recharged by connecting the hub 702a to a source of electrical power (e.g., via a cable), by wirelessly charging (e.g., via a wireless charging pad), by solar energy (e.g., via a solar collector if incorporated in the hub 702a), and/or by kinetic motion (e.g., via an internal mechanism if incorporated that can convert kinetic motion into electrical power). In some cases, the battery can be removed, or the battery can be integrated within and/or a permanent part of the hub 702a.

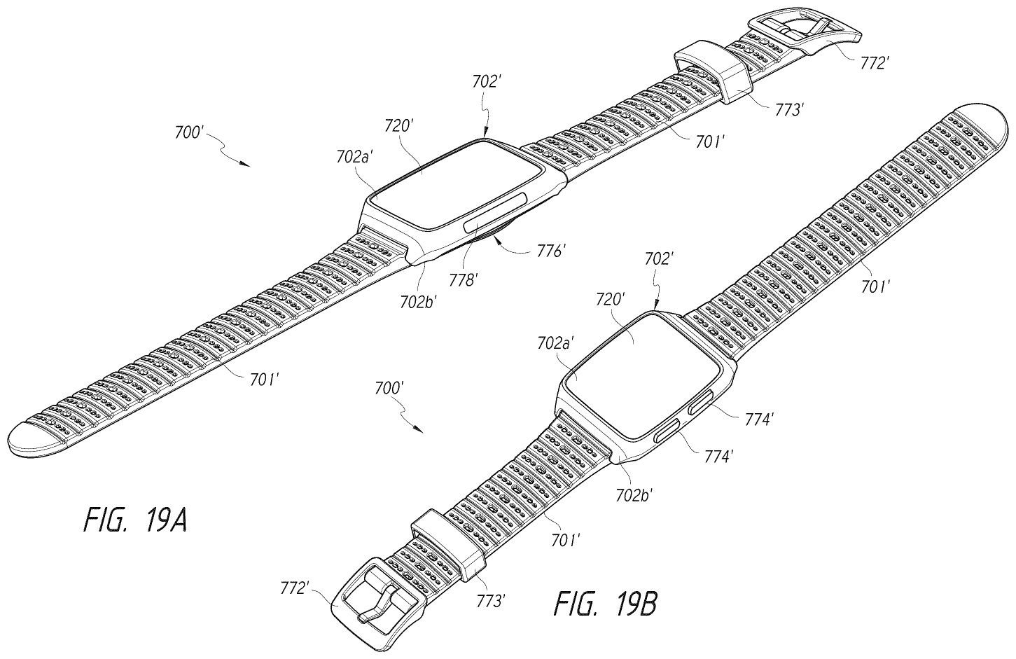

FIGS. 19A-19B illustrate a pulse oximetry system 700′ that is a variant of the pulse oximetry system 700 described and illustrated with respect to FIGS. 18A-18M. FIGS. 19A-19B illustrate top perspective views of the pulse oximetry system 700′. The pulse oximetry system 700′ can be similar to the pulse oximetry system 700 in some or many respects. The pulse oximetry system 700′ differs from the pulse oximetry system 700 in that its wrist portion 702′ can include a display 720′ similar or identical to the display of the wearable sensor described in U.S. Pat. Pub. No. US2021/0290120 incorporated by reference herein, which can be configured to display a plurality of physiological parameters monitored and/or determined by the pulse oximetry system 700′ and/or other information (e.g., such as the time, date, etc.). Further, the display 720′ can be configured to receive input from the subject, such as touch input, for interacting with the pulse oximetry system 700′. Such display 720′ can be disposed on a top of the wrist portion 702′ such that it can be accessible when the pulse oximetry system 700′ is worn by a subject. The pulse oximetry system 700′ also differs from the pulse oximetry system 700 in that its ECG electrode 778′ can be disposed along a side of the wrist portion 702′ such as shown in FIG. 19A. The pulse oximetry system 700′ can also differ from the pulse oximetry system 700 by including one or more user interfaces 774′ similar or identical to the user interfaces 13 (e.g., user interface 13a and/or user interface 13b) of the wearable sensor described in U.S. Pat. Pub. No. US2021/0290120 incorporated by reference herein, which can be configured as button(s). As shown in FIG. 19B, such user interfaces 774′ can be disposed along a side of the wrist portion 702′.

In some implementations, the wrist portion 702′ comprises a first component 702a′ (which may also be referred to as a “sensor hub” or “hub”) and a second component 702b′ (which may also be referred to as a “dock”), which can be similar to and removably secure to one another like the hub 702a and dock 702b of pulse oximetry system 700. In such implementations, the hub 702a′ can include the display 720′, the ECG electrode 778′, and the user interfaces 774′. Furthermore, in such implementations, the dock 702b′ can be configured to allow a subject to access the ECG electrode 778′ and user interfaces 774′ when the hub 702a′ and the dock 702b′ are secured to one another. For example, the dock 702b′ can include windows/through holes at/adjacent the locations of the ECG electrode 778′ and user interfaces 774′ to allow a subject access to such features (e.g., the dock 702b′ can have windows/through holes along its sides).

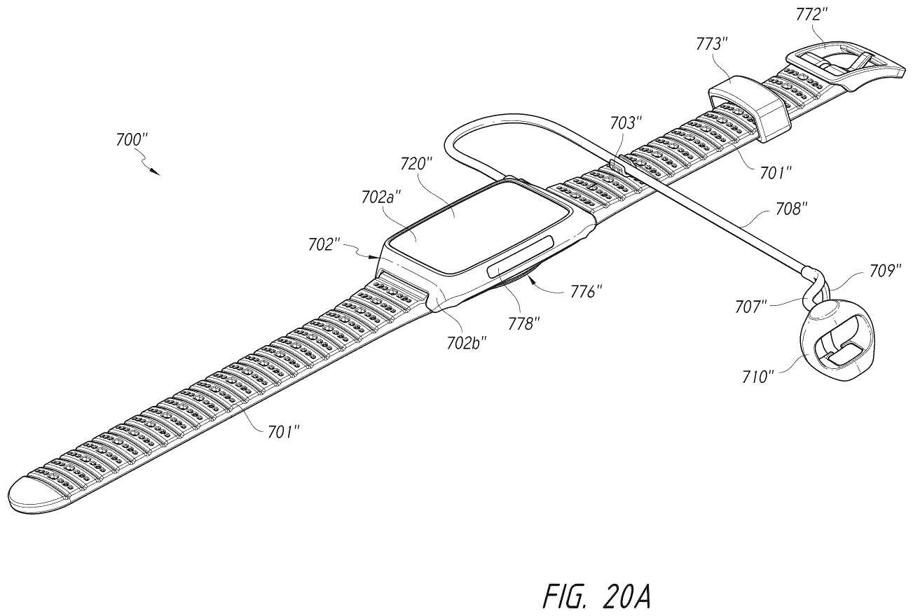

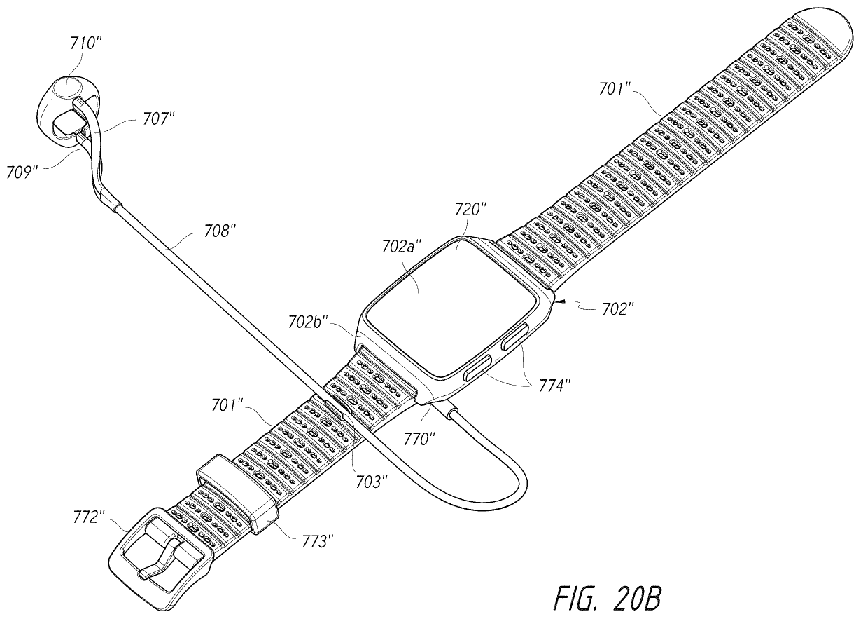

FIGS. 20A-20B illustrate a pulse oximetry system 700″ that is a variant of the pulse oximetry system 700′ described and illustrated with respect to FIGS. 19A-19B. FIGS. 20A-20B illustrate top perspective views of the pulse oximetry system 700″. The pulse oximetry system 700″ can be similar to the pulse oximetry system 700′ in some or many respects. The pulse oximetry system 700″ differs from the pulse oximetry system 700′ in that it includes a cable 708″ and a ring member 710″. Cable 708″ can be similar or identical to any of cables 108, 408, 508, and/or 608 discussed herein. Cable 708″ can split into first and second portions 707″, 709″ similar or identical to cables 108, 408, 508, and/or 608 discussed elsewhere herein. Similar to that discussed with respect to cables 108, 408, 508, and/or 608, cable 708″ can operably connect wrist portion 702″ (e.g., the hub 702a″ of the wrist portion 702″) with an emitter housing and/or emitter package comprising one or more emitters (similar and/or identical to the those described herein, such as emitter package 134 comprising one or more emitters 112) and a detector housing and/or a detector package comprising one or more detectors (similar and/or identical to the those described herein, such as detector package 136 comprising one or more detectors 114). Ring member 710″, which can operably position such emitter housing and/or emitter package and detector housing and/or detector package, can be similar or identical to any of the ring members discussed elsewhere herein, such as ring member 110, 410, 510, and/or 610.

In some implementations, the cable 708″ (and thus the one or more emitters and one or more detectors operably positioned by the ring member 710″) is detachable from the wrist portion 702″, for example, removably connectable to a connector port 770″ of the wrist portion 702″ (e.g., detachable from hub 702a″ at connector port 770″). In some implementations, cable 708″ is permanently attached to the wrist portion 702″ (e.g., permanently attached to hub 702a″). The connector port 770″ can be disposed at or adjacent a corner of a side of the wrist portion 702″ as shown in FIGS. 20A-20B, or it can be disposed along a side of the wrist portion 702″ (not shown). In some implementations, the wrist portion 770″ can include more than one connector port 770″ (for example, one on each side of the wrist portion 770″). The cable 708″ can be secured to and/or positioned along a subject similar or identical to that discussed with respect to cable 108 in pulse oximetry system 100, for example, with the utilization of one or more cable retainers (such as cable retainers 103, 105) and/or similar to that discussed above with respect to pulse oximetry system 100. In some implementations, pulse oximetry system 700″ includes a cable retainer 703″ that can be similar or identical to any of the cable retainers discussed herein, such as cable retainer 403, 503, and/or 603. Cable retainer 703″ can be permanently or removably attached to one of straps 701″.

In some implementations, physiological parameter measurement sensor or module 776″ can work in combination with the one or more emitters and one or more detectors operably positioned by ring member 710″. For example, signals and/or data outputted by the module 776″ can augment any or all signals and/or data outputted by the one or more detectors operably positioned by ring member 710″, such as if signals and/or data outputted by the one or more detectors is interrupted or lost. As another example, the signals and/or data outputted by module 776″ can validate any or all signals and/or data outputted by the one or more detectors operably positioned by ring member 710″. In a further example, the module 776″ can be used as a primary source of signals and/or data for determining one or more physiological parameters of a subject if the cable 708″ (and thus the one or more emitters and one or more detectors operably positioned by the ring member 710″) is detached from the wrist portion 702″. In some implementations, the module 776″ can measure physiological parameters of interest separate of and/or distinct from the one or more emitters and one or more detectors operably positioned by ring member 710″. In some implementations, the module 776″ can be automatically turned off and/or not used when the cable 708″ (and thus the one or more emitters and one or more detectors operably positioned by the ring member 710″) is connected to the wrist portion 702″ (e.g., such as connected at connector port 770″). For example, during the day and/or when out in public, a subject can wear the pulse oximetry system 700″ without the cable 708″ connected. During such use, the module 776″ can be used to measure physiological parameters of the subject. Such use can advantageously allow the subject to wear and use the pulse oximetry system in a concealed manner. During the night (e.g., when sleeping) and/or when not in public, a subject can wear the pulse oximetry system 700″ with the cable 708″ connected. During such use, the one or more emitters and one or more detectors operably positioned by the ring member 710″ can be used to measure physiological parameters of the subject. Furthermore, during such use, the module 776″ can be automatically turned off, not used, used to measure different physiological parameters than the emitters/detectors operably positioned by the ring member 710″, or used to augment measurements of the emitters/detectors operably positioned by the ring member 710″. For example, a processor of the wrist portion 702″ can determine whether cable 708″ is connected to the wrist portion 702″ (e.g., via port 770″) and disable operation of module 776″ when cable 708″ is connected, and enable/instruct operation of module 776″ when cable 708″ is not connected. Further to this example, a processor of the wrist portion 702″ can determine whether cable 708″ is connected to the wrist portion 702″ and modify operation of module 776″ when cable 708″ is connected, such as to measure different physiological parameters than the emitters/detectors operably positioned by the ring member 710″, or to augment measurements of the emitters/detectors operably positioned by the ring member 710″.

Although examples and certain orientations and configurations of various aspects of the pulse oximetry systems described in this disclosure (e.g., pulse oximetry systems 100, 400, 500, 500′, 600, 600′, 600″, 600″′, 700, 700′, 700″) have been provided, alternative orientations and configurations for such aspects are to be considered included as a part of this disclosure. For example, although certain orientations and configurations for the orientation/direction in which the docks described herein (e.g., docks 106 and 306) can be secured to a subject's wrist 3 have been provided, the docks described herein can be secured to a subject's wrist 3 in any orientation/direction. For example, the docks described herein (e.g., docks 106 and 306) can be secured to a subject's wrist with their arm(s) (e.g., arms(s) 122) directed in any direction, such as towards the palm 4, away from the palm 4, and/or any direction in between including perpendicular to the palm 4. With different orientations/directions in which the docks described herein can be secured to a subject's wrist, the orientation of the sensor hubs (e.g., sensor hub 102) with respect to the docks as well as the direction in which the sensor hubs may need to be slid/directed/pushed towards the docks (e.g., to connect the sensor hubs with the sensor docks) and/or away from the docks (e.g., to disconnect the sensor hubs from the sensor docks) may need to be changed as well, however the releasable mechanical and electrical connection between the docks and hubs can remain. As another example, although certain orientations and configurations for the orientation, direction, and/or routing of the cables 108, 308, 408, 508, 608, 708″ and any variations thereof and any of their portions if included (e.g., first portions 107, 407, 507, 607, and 707″ and variations thereof and second portions 109, 409, 509, 609, and 709″ and variations thereof) have been described, the cables 108, 308, 408, 508, 608, and 708″ and variations thereof and any of their portions can be oriented, directed, and/or routed in any other ways. For example, the cables 108, 308, 408, 508, 608, and 708″ and any variations thereof can be directed towards the palm 4, away from the palm 4, and/or any direction in between including perpendicular to the palm 4 when extending from their respective pulse oximetry systems or portions thereof, such as when extending from their respective bend reliefs (e.g., bend relief 120a) that connect the cables to their respective pulse oximetry system when included. As another example, although certain orientations and configurations for the orientation and position of connector ports, such as connector ports 570, 570′, 670, 670′, and 770″, and cables connected thereto have been described, the connector ports can be oriented, directed, and/or positioned in any other ways. For example, connector ports, where included, can be disposed at or adjacent a corner of a side of their associated system or disposed along a side of their associated system (e.g., of a wrist portion, a hub, and/or a dock of their associated system). In some implementations, the systems described herein can include more than one connector port, such as one on each side (including at or adjacent a corner or along the side) of a wrist portion, a hub, and/or a dock thereof.

The pulse oximetry systems described herein, such as the pulse oximetry systems 100, 400, 500, 600, 700 and any variations thereof and/or any of their components can be configured to be waterproof, water resistant, drip proof, shock proof, dust proof, and/or dust resistant. While the pulse oximetry systems have been described as having a rechargeable battery, the battery can be nonrechargeable or single use. In some implementations, a battery of the pulse oximetry system (such as battery 102d and/or the implementation of such a battery 165) can be rechargeable but non-removable from the system. In such a case, the pulse oximetry system can include a charge port configured to receive a power cable for charging. Further in such a case, the pulse oximetry system can be used by the subject while charging (e.g., the pulse oximetry system can be in an operational mode while charging). In some variants, a sensor hub of any of the pulse oximetry systems described herein (such as sensor hub 102) can be permanently connected to a dock of any of the pulse oximetry systems described herein (such as dock 106). In such configurations, the combined dock/sensor hub can have a charge port for charging.

In some implementations, any or all of the components of the pulse oximetry sensors as described herein can be configured to be reusable (which may also be referred to herein as “durable”). For example, in reference to the pulse oximetry system 100, the sensor hub 102 and the sensor dock assembly 104 including the dock 106, the cable 108, the emitter package 134 in emitter housing 130, the detector package 136 in detector housing 132, and the ring member 110 can all be configured to be reusable (e.g., for days, weeks, months, or more). In such a case, all components can be sanitized between uses and/or between subjects. In some implementations, all components of the pulse oximetry systems as described herein can be configured to be reusable except for the ring members as described herein, such as ring members 110 and 410. In some cases, all components of the pulse oximetry systems as described herein can be configured to be reusable between subjects except for the ring members as described herein, such as ring members 110 and 410 (e.g., subjects do not share use of a ring member). In some implementations, the sensor hubs as described herein, such as sensor hub 102, last longer than all other components of the pulse oximetry system and can be reused if desired between subjects. In some implementations, one or more components of the pulse oximetry systems as described herein, such as a ring member (e.g., ring members 110 and 410), a clip member (e.g., clip member 310), a cable (e.g., cables 108, 308, and 408) and any portions thereof, a dock (e.g., docks 104 and 304) and any portions thereof, and sensor components (e.g., emitter packages 134 and 334 and detector packages 136 and 336) and any portions thereof, can be configured as single use (which may be referred to herein as “disposable”). In such a configuration, the sensor hubs as described herein can be integrated, a part of, and/or otherwise combined with the docks as described herein to provide for a single and fully integrated component that can be secured to the subject's wrist 3. Furthermore, in such a configuration, the pulse oximetry systems can include a single use battery and/or non-rechargeable battery (e.g., a zinc-air battery). In some implementations, all components of the pulse oximetry systems as described herein can be configured to be single use except for the sensor hubs (e.g., sensor hub 102).

### Additional Considerations and Terminology

Certain categories of persons, such as caregivers, clinicians, doctors, nurses, and friends and family of a subject, may be used interchangeably to describe a person providing care to the subject. Furthermore, subjects, patients, or users used herein interchangeably refer to a person who is wearing a sensor or is connected to a sensor or whose measurements are used to determine a physiological parameter or a condition. Parameters may be, be associated with, and/or be represented by, measured values, display icons, alphanumeric characters, graphs, gauges, power bars, trends, or combinations. Real time data may correspond to active monitoring of a subject, however, such real time data may not be synchronous to an actual physiological state at a particular moment. Measurement value(s) of a parameter such as any of those discussed herein, unless specifically stated otherwise, or otherwise understood with the context as used is generally intended to convey a measurement or determination that is responsive to and/or indicative of the physiological parameter.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more implementations necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each”is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 10 degrees, 5 degrees, 3 degrees, or 1 degree. As another example, in certain implementations, the terms “generally perpendicular” and “substantially perpendicular” refer to a value, amount, or characteristic that departs from exactly perpendicular by less than or equal to 10 degrees, 5 degrees, 3 degrees, or 1 degree.

Although certain implementations and examples have been described herein, it will be understood by those skilled in the art that many aspects of the systems and devices shown and described in the present disclosure may be differently combined and/or modified to form still further implementations or acceptable examples. All such modifications and variations are intended to be included herein within the scope of this disclosure. A wide variety of designs and approaches are possible. No feature, structure, or step disclosed herein is essential or indispensable.

Any methods disclosed herein need not be performed in the order recited. The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, and/or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

Depending on the implementation, certain acts, events, or functions of any of the processes or algorithms described herein can be performed in a different sequence, can be added, merged, or left out altogether (for example, not all described operations or events are necessary for the practice of the algorithm). Moreover, in certain implementations, operations or events can be performed concurrently, e.g., through multi-threaded processing, interrupt processing, or multiple processors or processor cores or on other parallel architectures, rather than sequentially.

Various illustrative logical blocks, modules, routines, and algorithm steps that may be described in connection with the disclosure herein can be implemented as electronic hardware (e.g., ASICs or FPGA devices), computer software that runs on general purpose computer hardware, or combinations of both. Various illustrative components, blocks, and steps may be described herein generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, various illustrative logical blocks and modules that may be described in connection with the disclosure herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. A processor can include an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of any method, process, routine, or algorithm described in connection with the disclosure herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. A pulse oximetry system comprising:
a wrist portion configured for placement on a wrist of a subject, the wrist portion comprising a first component and a second component configured to removably secure to one another, the first component comprising one or more hardware processors and a battery, the second component comprising a physiological parameter measurement module operably positioned at a bottom of the second component that is configured to be positioned proximate the subject's wrist, the physiological parameter measurement module comprising at least one emitter configured to emit optical radiation into tissue of the subject's wrist and at least one detector configured to detect at least a portion of the optical radiation reflected from said tissue, the at least one detector further configured to output at least one signal responsive to the detected optical radiation; and
at least one strap configured to secure the wrist portion to the subject's wrist, said at least one strap coupled to the second component;
wherein the one or more hardware processors of the first component are configured to receive and process said at least one signal to determine at least one physiological parameter of the subject when the first component and the second component are secured to one another.

2. The pulse oximetry system of claim 1, wherein the first component is further configured to wirelessly transmit said one or more physiological parameters to a separate device.

3. The pulse oximetry system of claim 1, wherein the first component does not comprise a display.

4. The pulse oximetry system of claim 1, further comprising:
a finger portion configured to secure to a finger of the subject, the finger portion comprising at least one emitter configured to emit optical radiation into tissue of the subject's finger and at least one detector configured to detect at least a portion of the optical radiation after passing through the tissue of the subject's finger, the at least one detector of the finger portion further configured to output at least one signal responsive to the detected optical radiation; and
a cable electrically connecting the finger portion and the wrist portion together, the cable configured to transmit said at least one signal outputted by the at least one detector of the finger portion to the wrist portion;
wherein the one or more hardware processors of the first component are configured to receive and process said at least one signal from the at least one detector of the finger portion to determine at least one physiological parameter of the subject when the first component and the second component are secured to one another.

5. The pulse oximetry system of claim 1, wherein the first component and the second component are configured to removably secure to one another via a latch mechanism.

6. The pulse oximetry system of claim 5, wherein the latch mechanism includes a protrusion that, in a resting state of the latch mechanism, extends outward from the first component that is configured to engage with a cavity of the second component to connect the first and second components to one another.

7. The pulse oximetry system of claim 1, wherein:
the first component comprises one or more electrical connectors and the second component comprises one or more electrical connectors; and
the one or more electrical connectors of the first component are configured to electrically connect with the one or more electrical connectors of the second component when the first and second components are secured to one another; and
the one or more electrical connectors of the first and second components are configured to facilitate communication between the first and second components.

8. The pulse oximetry system of claim 1, wherein the first component comprises a rechargeable battery.

9. The pulse oximetry system of claim 1, wherein the second component does not include a battery.

10. The pulse oximetry system of claim 1, wherein the first component comprises a motion sensor.

11. The pulse oximetry system of claim 1, wherein the second component comprises an electrocardiogram (ECG) sensor positioned at the bottom of the second component.

12. The pulse oximetry system of claim 11, wherein the ECG sensor comprises a first ECG electrode and a second ECG electrode, the first and second ECG electrodes configured to contact skin of the subject when the pulse oximetry system is secured to the subject's wrist.

13. The pulse oximetry system of claim 1, wherein said at least one strap comprises two straps.

14. The pulse oximetry system of claim 13, wherein the straps are permanently coupled to the second component.
